SEQUENCE LISTING (38 sequences)

SEQ ID NO: 1 - protein, 68 aa, Naja kaouthia
IRCFITPDITSKDCPNGHVCYTKTWCDAFCSIRGKRVDLGCAATCPTVKTGVDIQCCSTDNCNPFPTR

SEQ ID NO: 2 - protein, 465 aa, Artificial Sequence
  Mtalpha-cobratoxinc7HopQ
  misc_feature (15)..(15): Xaa can be any naturally occurring amino acid
  misc_feature (403)..(403): Xaa can be any naturally occurring amino acid
IRCFITPDITSKDCXKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKXGHVCYTKTWCDAFCSIRGKRVDLGCAATCPTVKTGVDIQCCSTDNCNPFPTRHHHHHHEPEA

SEQ ID NO: 3 - protein, 73 aa, Bungarus multicinctus
IVCHTTATSPISAVTCPPGENLCYRKMWCDVFCSSRGKVVELGCAATCPSKKPYEEVTCCSTDKCNPHPKQRP

SEQ ID NO: 4 - protein, 471 aa, Artificial Sequence
  Mtalpha-bungarotoxinc7HopQ
  misc_feature (18)..(18): Xaa can be any naturally occurring amino acid
  misc_feature (406)..(406): Xaa can be any naturally occurring amino acid
IVCHTTATSPISAVTCPXKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKXENLCYRKMWCDVFCSSRGKVVELGCAATCPSKKPYEEVTCCSTDKCNPHPKQRPGHHHHHHEPEA

SEQ ID NO: 5 - protein, 760 aa, Escherichia coli
NADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQ

SEQ ID NO: 6 - protein, 850 aa, Artificial Sequence
  MtAlpha-cobratoxinc2YgjkQ randomlinkers
  misc_feature (15)..(15): Xaa can be any naturally occurring amino acid
  misc_feature (788)..(788): Xaa can be any naturally occurring amino acid
IRCFITPDITSKDCXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXGHVCYTKTWCDAFCSIRGKRVDLGCAATCPTVKTGVDIQCCSTDNCNPFPTRHHHHHHEPEA

SEQ ID NO: 7 - protein, 851 aa, Artificial Sequence
  MtAlpha-cobratoxinc2YgjkQ randomlinkers
  misc_feature (15)..(15): Xaa can be any naturally occurring amino acid
  misc_feature (788)..(789): Xaa can be any naturally occurring amino acid
IRCFITPDITSKDCXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXXGHVCYTKTWCDAFCSIRGKRVDLGCAATCPTVKTGVDIQCCSTDNCNPFPTRHHHHHHEPEA

SEQ ID NO: 8 - protein, 851 aa, Artificial Sequence
  MtAlpha-cobratoxinc2YgjkQ randomlinkers
  misc_feature (15)..(16): Xaa can be any naturally occurring amino acid
  misc_feature (789)..(789): Xaa can be any naturally occurring amino acid
IRCFITPDITSKDCXXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXGHVCYTKTWCDAFCSIRGKRVDLGCAATCPTVKTGVDIQCCSTDNCNPFPTRHHHHHHEPEA

SEQ ID NO: 9 - protein, 852 aa, Artificial Sequence
  MtAlpha-cobratoxinc2YgjkQ randomlinkers
  misc_feature (15)..(16): Xaa can be any naturally occurring amino acid
  misc_feature (789)..(790): Xaa can be any naturally occurring amino acid
IRCFITPDITSKDCXXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXXGHVCYTKTWCDAFCSIRGKRVDLGCAATCPTVKTGVDIQCCSTDNCNPFPTRHHHHHHEPEA

SEQ ID NO: 10 - protein, 17 aa, Artificial Sequence
  cYgjk circular permutation linker peptide
ASGGGSGGGGSGGGGSG

SEQ ID NO: 11 - protein, 64 aa, Micrurus mipartitus
LTCKTCPFTTCPNSESCPGGQSICYQRKWEEHRGERIERRCVANCPAFGSHDTSLLCCTRDNCN

SEQ ID NO: 12 - protein, 846 aa, Artificial Sequence
  Mtmicrurotoxin1c2YgjK randomlinkers
  misc_feature (19)..(19): Xaa can be any naturally occurring amino acid
  misc_feature (792)..(792): Xaa can be any naturally occurring amino acid
LTCKTCPFTTCPNSESCPXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXQSICYQRKWEEHRGERIERRCVANCPAFGSHDTSLLCCTRDNCNHHHHHHEPEA

SEQ ID NO: 13 - protein, 847 aa, Artificial Sequence
  Mtmicrurotoxin1c2YgjK randomlinkers
  misc_feature (19)..(19): Xaa can be any naturally occurring amino acid
  misc_feature (792)..(793): Xaa can be any naturally occurring amino acid
LTCKTCPFTTCPNSESCPXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXXQSICYQRKWEEHRGERIERRCVANCPAFGSHDTSLLCCTRDNCNHHHHHHEPEA

SEQ ID NO: 14 - protein, 847 aa, Artificial Sequence
  Mtmicrurotoxin1c2YgjK randomlinkers
  misc_feature (19)..(20): Xaa can be any naturally occurring amino acid
  misc_feature (793)..(793): Xaa can be any naturally occurring amino acid
LTCKTCPFTTCPNSESCPXXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXQSICYQRKWEEHRGERIERRCVANCPAFGSHDTSLLCCTRDNCNHHHHHHEPEA

SEQ ID NO: 15 - protein, 848 aa, Artificial Sequence
  Mtmicrurotoxin1c2YgjK randomlinkers
  misc_feature (19)..(20): Xaa can be any naturally occurring amino acid
  misc_feature (793)..(794): Xaa can be any naturally occurring amino acid
LTCKTCPFTTCPNSESCPXXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXXQSICYQRKWEEHRGERIERRCVANCPAFGSHDTSLLCCTRDNCNHHHHHHEPEA

SEQ ID NO: 16 - protein, 428 aa, Heliobacter pylori
MAVQKVKNADKVQKLSDTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKYQQDNQTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNPFRNMGMIASSTTNNGA

SEQ ID NO: 17 - protein, 855 aa, Artificial Sequence
  MtBgTXc2Ygjk randomlinkers
  misc_feature (18)..(18): Xaa can be any naturally occurring amino acid
  misc_feature (791)..(791): Xaa can be any naturally occurring amino acid
IVCHTTATSPISAVTCPXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXENLCYRKMWCDVFCSSRGKVVELGCAATCPSKKPYEEVTCCSTDKCNPHPKQRPHHHHHHEPEA

SEQ ID NO: 18 - protein, 856 aa, Artificial Sequence
  MtBgTXc2Ygjk randomlinkers
  misc_feature (18)..(19): Xaa can be any naturally occurring amino acid
  misc_feature (792)..(792): Xaa can be any naturally occurring amino acid
IVCHTTATSPISAVTCPXXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXENLCYRKMWCDVFCSSRGKVVELGCAATCPSKKPYEEVTCCSTDKCNPHPKQRPHHHHHHEPEA

SEQ ID NO: 19 - protein, 856 aa, Artificial Sequence
  MtBgTXc2Ygjk randomlinkers
  misc_feature (18)..(18): Xaa can be any naturally occurring amino acid
  misc_feature (791)..(792): Xaa can be any naturally occurring amino acid
IVCHTTATSPISAVTCPXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXXENLCYRKMWCDVFCSSRGKVVELGCAATCPSKKPYEEVTCCSTDKCNPHPKQRPHHHHHHEPEA

SEQ ID NO: 20 - protein, 857 aa, Artificial Sequence
  MtBgTXc2Ygjk randomlinkers
  misc_feature (18)..(19): Xaa can be any naturally occurring amino acid
  misc_feature (792)..(793): Xaa can be any naturally occurring amino acid
IVCHTTATSPISAVTCPXXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXXENLCYRKMWCDVFCSSRGKVVELGCAATCPSKKPYEEVTCCSTDKCNPHPKQRPHHHHHHEPEA

SEQ ID NO: 21 - protein, 461 aa, Artificial Sequence
  MtMmTX1c7HopQ
  misc_feature (19)..(19): Xaa can be any naturally occurring amino acid
  misc_feature (407)..(407): Xaa can be any naturally occurring amino acid
LTCKTCPFTTCPNSESCPXTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSXQSICYQRKWEEHRGERIERRCVANCPAFGSHDTSLLCCTRDNCNHHHHHHEPEA

SEQ ID NO: 22 - protein, 751 aa, Artificial Sequence
  MtBgTXc7HopQ-Aga2p_ACP protein sequence
  misc_feature (107)..(107): Xaa can be any naturally occurring amino acid
  misc_feature (495)..(495): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAIVCHTTATSPISAVTCPXKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKXENLCYRKMWCDVFCSSRGKVVELGCAATCPSKKPYEEVTCCSTDKCNPHPKQRPGSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 23 - protein, 848 aa, Artificial Sequence
  MtMmTX1c1YgjK randomlinkers
  misc_feature (19)..(19): Xaa can be any naturally occurring amino acid
  misc_feature (794)..(794): Xaa can be any naturally occurring amino acid
LTCKTCPFTTCPNSESCPXKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKXQSICYQRKWEEHRGERIERRCVANCPAFGSHDTSLLCCTRDNCNHHHHHHEPEA

SEQ ID NO: 24 - protein, 847 aa, Artificial Sequence
  MtMmTX1c1YgjK randomlinkers
  misc_feature (19)..(19): Xaa can be any naturally occurring amino acid
  misc_feature (793)..(793): Xaa can be any naturally occurring amino acid
LTCKTCPFTTCPNSESCPXEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKXQSICYQRKWEEHRGERIERRCVANCPAFGSHDTSLLCCTRDNCNHHHHHHEPEA

SEQ ID NO: 25 - protein, 847 aa, Artificial Sequence
  MtMmTX1c1YgjK randomlinkers
  misc_feature (19)..(19): Xaa can be any naturally occurring amino acid
  misc_feature (793)..(793): Xaa can be any naturally occurring amino acid
LTCKTCPFTTCPNSESCPXKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVXQSICYQRKWEEHRGERIERRCVANCPAFGSHDTSLLCCTRDNCNHHHHHHEPEA

SEQ ID NO: 26 - protein, 846 aa, Artificial Sequence
  MtMmTX1c1YgjK randomlinkers
  misc_feature (19)..(19): Xaa can be any naturally occurring amino acid
  misc_feature (792)..(792): Xaa can be any naturally occurring amino acid
LTCKTCPFTTCPNSESCPXEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVXQSICYQRKWEEHRGERIERRCVANCPAFGSHDTSLLCCTRDNCNHHHHHHEPEA

SEQ ID NO: 27 - protein, 175 aa, Stichodactyla helianthus [sequence omitted]

SEQ ID NO: 28 - protein, 572 aa, Artificial Sequence
  MtStIIc7HopQ randomlinkers
  misc_feature (92)..(92): Xaa can be any naturally occurring amino acid
  misc_feature (480)..(480): Xaa can be any naturally occurring amino acid
ALAGTIIAGASLTFQVLDKVLEELGKVSRKIAVGIDNESGGTWTALNAYFRSGTTDVILPEFVPNTKALLYSGRKDTGPVATGAVAAFAYYXTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSXSGNTLGVMFSVPFDYNWYSNWWDVKIYSGKRRADQGMYEDLYYGNPYRGDNGWHEKNLGYGLRMKGIMTSAGEAKMQIKISRHHHHHHEPEA

SEQ ID NO: 29 - protein, 957 aa, Artificial Sequence
  MtStIIc1YgjK randomlinkers
  misc_feature (92)..(92): Xaa can be any naturally occurring amino acid
  misc_feature (865)..(865): Xaa can be any naturally occurring amino acid
ALAGTIIAGASLTFQVLDKVLEELGKVSRKIAVGIDNESGGTWTALNAYFRSGTTDVILPEFVPNTKALLYSGRKDTGPVATGAVAAFAYYXEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVXSGNTLGVMFSVPFDYNWYSNWWDVKIYSGKRRADQGMYEDLYYGNPYRGDNGWHEKNLGYGLRMKGIMTSAGEAKMQIKISRHHHHHHEPEA

SEQ ID NO: 30 - protein, 267 aa, Ricinus communis
IFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQF

SEQ ID NO: 31 - protein, 664 aa, Artificial Sequence
  MtRTA36-302c7HopQ
  misc_feature (65)..(65): Xaa can be any naturally occurring amino acid
  misc_feature (453)..(453): Xaa can be any naturally occurring amino acid
IFPKQYPIINFTTAGATVQSYTNFIRAVRGRLTTGADVRHEIPVLPNRVGLPINQRFILVELSNXKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKXELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFHHHHHHEPEA

SEQ ID NO: 32 - protein, 1136 aa, Artificial Sequence
  MtBgTxc2YgjK-Aga2p_ACP protein sequence
  misc_feature (107)..(107): Xaa can be any naturally occurring amino acid
  misc_feature (880)..(880): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAIVCHTTATSPISAVTCPXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXENLCYRKMWCDVFCSSRGKVVELGCAATCPSKKPYEEVTCCSTDKCNPHPKQRPGSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 33 - protein, 1137 aa, Artificial Sequence
  MtBgTxc2YgjK
  misc_feature (107)..(108): Xaa can be any naturally occurring amino acid
  misc_feature (881)..(881): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAIVCHTTATSPISAVTCPXXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXENLCYRKMWCDVFCSSRGKVVELGCAATCPSKKPYEEVTCCSTDKCNPHPKQRPGSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 34 - protein, 1137 aa, Artificial Sequence
  MtBgTxc2YgjK
  misc_feature (107)..(107): Xaa can be any naturally occurring amino acid
  misc_feature (880)..(881): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAIVCHTTATSPISAVTCPXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXXENLCYRKMWCDVFCSSRGKVVELGCAATCPSKKPYEEVTCCSTDKCNPHPKQRPGSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 35 - protein, 1138 aa, Artificial Sequence
  MtBgTxc2YgjK
  misc_feature (107)..(108): Xaa can be any naturally occurring amino acid
  misc_feature (881)..(882): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAIVCHTTATSPISAVTCPXXQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLXXENLCYRKMWCDVFCSSRGKVVELGCAATCPSKKPYEEVTCCSTDKCNPHPKQRPGSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 36 - protein, 129 aa, Ricinus communis
QVQLVESGGGIVQPGGSLRLSCAASGFTLDDYAIGWFRQVPGKEREGVACVKDGSTYYADSVKGRFTISRDNGAVYLQMNSLKPEDTAVYYCASRPCFLGVPLIDFGSWGQGTQVTVSSSAWSHPQFEK

SEQ ID NO: 37 - protein, 64 aa, Tityus serrulatus
KEGYLMDHEGCKLSCFIRPSGYCGRECGIKKGSSGYCAWPACYCYGLPNWVKVWDRATNKCGKK

SEQ ID NO: 38 - protein, 844 aa, Artificial Sequence
  MtTs1c1YgjK
  misc_feature (38)..(38): Xaa can be any naturally occurring amino acid
  misc_feature (812)..(812): Xaa can be any naturally occurring amino acid
KEGYLMDHEGCKLSCFIRPSGYCGRECGIKKGSSGYCXKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVXPACYCYGLPNWVKVWDRATNKCHHHHHHEPEA

(19) United States
(12) Patent Application Publication
(10) Pub. No.: US 2022/0073574 A1
(43) Pub. Date: Mar. 10, 2022

(54) FUSION PROTEIN WITH A TOXIN AND SCAFFOLD PROTEIN

(71) Applicants: VIB VZW, Gent (BE); VRIJE UNIVERSITEIT BRUSSEL, Brussel (BE)

(72) Inventors: Jan Steyaert, Beersel (BE); Els Pardon, Wezemaal (BE); Wim Vranken, Brussel (BE)

(21) Appl. No.: 17/415,461

(22) PCT Filed: Dec. 20, 2019
(86) PCT No.: PCT/EP2019/086717

§ 371 Date: Jun. 17, 2021

(30) Foreign Application Priority Data
Dec. 21, 2018 (EP) 18215677.8

(51) Int. Cl. C07K 14/435; G16B 15/20

(52) U.S. Cl. CPC C07K 14/43522; C07K 2319/55; G16B 15/20; C07K 14/43563

(57) ABSTRACT

The present invention relates to the field of structural biology and drug discovery. More specifically, the present invention relates to novel fusion proteins, their uses and methods in three-dimensional structural analysis of macromolecules, such as X-ray crystallography and high-resolution Cryo-EM, and their use in structure-based drug design and screening, and as pharmacological tools. Even more specifically, the invention relates to a functional fusion of a toxin and a scaffold protein wherein the folded scaffold protein interrupts the topology of the toxin by insertion in an exposed β-turn of a β-strand-containing domain of said toxin to form a rigid fusion protein that retains its high affinity target binding capacity.

### CROSS-REFERENCE TO RELATED APPLICATIONS

This application is a national phase entry under 35 U.S.C. § 371 of International Patent Application PCT/EP2019/086717, filed Dec. 20, 2019, designating the United States of America and published in English as International Patent Publication WO 2020/127993 on Jun. 25, 2020, which claims the benefit under Article 8 of the Patent Cooperation Treaty to European Patent Application Serial No. 18215677.8, filed Dec. 21, 2018, the entireties of which are hereby incorporated by reference.

### FIELD OF THE INVENTION

The present invention relates to the field of structural biology and drug discovery. More specifically, the present invention relates to novel fusion proteins, their uses and methods in three-dimensional structural analysis of macromolecules, such as X-ray crystallography and high-resolution Cryo-EM, and their use in structure-based drug design and screening, and as pharmacological tools. Even more specifically, the invention relates to a functional fusion of a toxin and a scaffold protein wherein the folded scaffold protein interrupts the topology of the toxin by insertion in an exposed β-turn of a β-strand-containing domain of said toxin to form a rigid fusion protein that retains its high affinity target binding capacity.

### BACKGROUND

The 3D-structural analysis of many proteins and complexes in certain conformational states remains difficult. Macromolecular X-ray crystallography intrinsically holds several disadvantages, such as the prerequisite for high quality purified protein, the relatively large amounts of protein that are required, and the preparation of diffraction quality crystals. The application of crystallization chaperones in the form of antibody fragments or other proteins has been proven to facilitate obtaining well-ordered crystals by minimizing the conformational heterogeneity in the target. Additionally, the chaperone can provide initial model-based phasing information (Koide, 2009). Still, single particle electron cryomicroscopy (cryo-EM) has recently developed into an alternative and versatile technique for structural analysis of macromolecular complexes at atomic resolution (Nogales, 2016). Although instrumentation and methods for data analysis improve steadily, the highest achievable resolution of the 3D reconstruction is mostly dependent on the homogeneity of a given sample, and the ability to iteratively refine the orientation parameters of each individual particle to high accuracy. Preferred particle orientation due to surface properties of the macromolecules that cause specific regions to preferentially adhere to the air-water interface or substrate support represent a recurring issue in cryo-EM. So also in this aspect, we are still missing tools such as next generation chaperones to overcome these hurdles.

Natural toxins are chemical agents of biological origin (including chemical agents and proteins) and can be produced by all types of organisms. Enzymatic and non-enzymatic proteins and peptides are the major toxin components, often present in animal venoms, many of which can target various ion channels, receptors, and membrane transporters. Compared to traditional small molecule drugs, toxins that are natural proteins and peptides exhibit higher specificity and potency to their targets. Toxins synthesized by venomous animals from both terrestrial animals and marine animals, such as scorpions, snakes, spiders, bees, cone snails, and sea anemones, are injected into the body for hunt or defense by animal wounding apparatus, such as fangs, barbs, spines, and stingers. Some venomous animals have been used to treat diseases for millennia in many parts of the world. Scorpion venom, as an example, has been used to treat spasms and endogenous wind in traditional Chinese medicine.

Venom toxins are highly potent short peptides or small proteins that are present in limited amounts in the venoms of various unrelated species, such as animals of the genus Conus (cone snails), arthropods (spiders, scorpions, centipedes, bees, etc.), vertebrates (snakes, lizards, etc.), and cnidarians (jellyfishes, sea anemones, etc.), insects, and worms amongst other animals (Mouhat et al., 2004). Venom toxins include at least four major classes of toxin, namely necrotoxins and cytotoxins, which kill cells; neurotoxins, which affect nervous systems; and myotoxins, which damage muscles.

Many of these toxins have been used extensively as biochemical and pharmacological tools to characterize and discriminate between various types of target proteins, such as ion-channels (voltage-gated and ligand-gated) or 7-transmembrane receptors, or G-protein coupled receptors (GPCR) as well as transporters, that differ in ionic selectivity, structure and/or cell function, and as such are of significant interest to the pharmaceutical and biotech industries as both therapeutic leads and pharmacological tools.

The peptide or small protein toxins have evolved over time on the basis of clearly distinct disulphide bridge frameworks and structural motifs, in order to adapt to different ion channel modulating strategies. Indeed, these toxins are structured by a high number of disulphide bridges (from two to five or more) in relation to their backbone length, thereby conferring rigidity to the molecules, a stabilization of their secondary structures, as well as a relative resistance to denaturation (heat, acid/alkali, detergents, etc.). For example, the Inhibitor cystine knot (ICK or also called Knottin) protein motif provides for a knot structure comprising at least 3 disulphide bridges and is very common in invertebrate toxins such as those from arachnids and molluscs. The motif is also found in some inhibitor proteins found in plants. The ICK motif is a very stable protein structure which is resistant to heat denaturation and proteolysis. Engineered knottins have shown significant promise as therapeutics, imaging agents, and targeting agents for chemotherapy. Indeed, immune cells express various voltage-gated and ligand-gated ion channels that mediate the influx and efflux of charged ions across the plasma membrane, thereby controlling the membrane potential and mediating intracellular signal transduction pathways. These channels thus present potential targets for experimental modulation of immune responses and for therapeutic interventions in immune disease. Small molecule drugs and natural toxins acting on such ion channels have illustrated the potential therapeutic benefit of targeting ion channels on immune cells. Though the application of immunotoxins in oncology studies copes with several issues such as the high immunogenicity.

Other examples include peptidergic toxins produced by snails, scorpions and spiders. Despite reported issues with manufacturability and stability, several toxin-derived peptides have advanced towards the clinic. For example, recently completed clinical studies with ShK-168 (Dalazatide), a K⁺ channel blocking sea anemone toxin variant, have shown lasting improvement of psoriasis lesions with an acceptable toxicity and immunogenicity profile. Ziconotide, a 25-amino acid Ca²⁺-channel blocking peptide derived from a snail toxin, is in the clinic for treatment of severe pain in terminal cancer patients.

The application of animal toxins as potential drug candidates in the treatment of human diseases, including cancer, neurodegenerative diseases, cardiovascular diseases, neuropathic pain, as well as autoimmune diseases, still faces a number of obstacles to translate new toxin discovery to their clinical applications. Challenges, strategies, and perspectives in the development of the protein toxin-based drugs are discussed for instance in Chen et al. (2018). The main drawbacks of small protein toxins as therapeutic agents are that they are highly difficult to isolate in a certain amount from extremely limited supplies of venom, since they are disulphide-bridge-rich gene engineering and chemical synthesis remain expensive and uncertain to yield enough bioactive products, as well as their short serum half-lives limiting their final efficacy to their targets in the treatment of diseases.

One structural superfamily largely distributed in Metazoans and several vertebrates is formed by the Three-finger fold toxin proteins, characterized by a short peptidic chain (60-80 residues) and a high content of disulphide bridges (4 to 5, sometimes 3-6). In fact, those toxins involve miniproteins frequently found in Elapidae snake venoms (Kessler et al., 2017). Their structural fold is characterized by three distinct loops rich in β-strands and emerging from a dense, globular core reticulated by four highly conserved disulphide bridges. The number and diversity of receptors, channels, and enzymes identified as targets of three-finger fold toxins is increasing continuously. Snake venom toxins belonging to the three-finger fold superfamily are able to trigger and recognize a wide variety of molecular targets though. Several three-finger fold toxins block the activity of the nicotinic and muscarinic acetylcholine receptors or inhibit the enzyme acetylcholinesterase and have become powerful pharmacological tools for studying the function and structure of their molecular targets. Other three-finger fold toxins, like micrurotoxin1 (MmTX1) and MmTX2, present in Costa Rican coral snake venom that tightly bind to the γ-aminobutyric acid receptors type-A (GAB_{A}A receptors, pentameric ligand-gated ion channels) at subnanomolar concentrations (Rosso et al., 2015). MmTX1 and MmTX2 allosterically increase GABA_{A} receptor susceptibility to agonist, thereby potentiating receptor opening as well as desensitization, possibly by interacting with the α+/β interface. The Charybdotoxin family of scorpion toxins is another example of a group of small peptides that has many family members. Some are pore-blocking toxins of eukaryotic voltage-dependent K⁺ channels (Banerjee et al., 2013).

Venom toxins are peptidic in nature, demonstrate high affinity for their targets, and are stable enough to resist fairly well degradation by proteases present in venoms and target tissues, which make them a unique source of lead compounds and templates for therapeutic drug discovery. Although it is clear that venoms constitute hundreds of peptide-based toxins that together encompass a high degree of stereochemical diversity, only a small fraction of these peptides or small proteins has been addressed in pharmacological studies so far. Structure-activity relationships of representative members and their targets is beneficial to decipher molecular determinants that permit these interactions with therapeutically relevant receptors and enzymes. High-resolution structural analysis would require that those small toxin proteins or peptides are chaperoned by chaperone molecules, which aid in adding mass, as well as in stabilizing certain conformational states or binding sites in complex with their targets. Finally, novel ways of engineering toxin proteins may create new avenues for therapeutic application of ‘engineered’ natural toxin targets.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A and 1B. Flexible fusion proteins compared to rigid toxin fusion proteins

(FIG. 1A) Flexible fusions or linkers at the N- or C-terminal end of a toxin and a scaffold protein using only one direct fusion or linker. (FIG. 1B) Rigid fusions of a toxin and a scaffold protein, wherein a toxin domain is fused with the scaffold protein via at least two direct fusions or linkers that connect a toxin domain to scaffold. The toxin used in this example is a three-finger fold toxin as found in for instance many snake venoms.

FIG. 2. Engineering principles of a toxin fusion protein built from a circularly permutated variant of a scaffold protein that is inserted into the β-turn connecting β-strands β2 and β3 of a three-finger fold toxin

This scheme shows how a toxin can be grafted onto a large scaffold protein via two peptide bonds or two short linkers that connect the toxin to the scaffold. Scissors indicate which exposed turns have to be cut in the toxin and in the scaffold. Dashed lines indicate how the remaining parts of the toxin and the scaffold have to be concatenated by use of peptide bonds or short peptide linkers to build the toxin fusion protein.

FIGS. 3A-3C. Model of a 50 kDa alpha-cobratoxin fusion protein built from a circularly permutated variant of HopQ inserted into the β-turn connecting β-strands 132 and 133 of the alpha-cobratoxin.

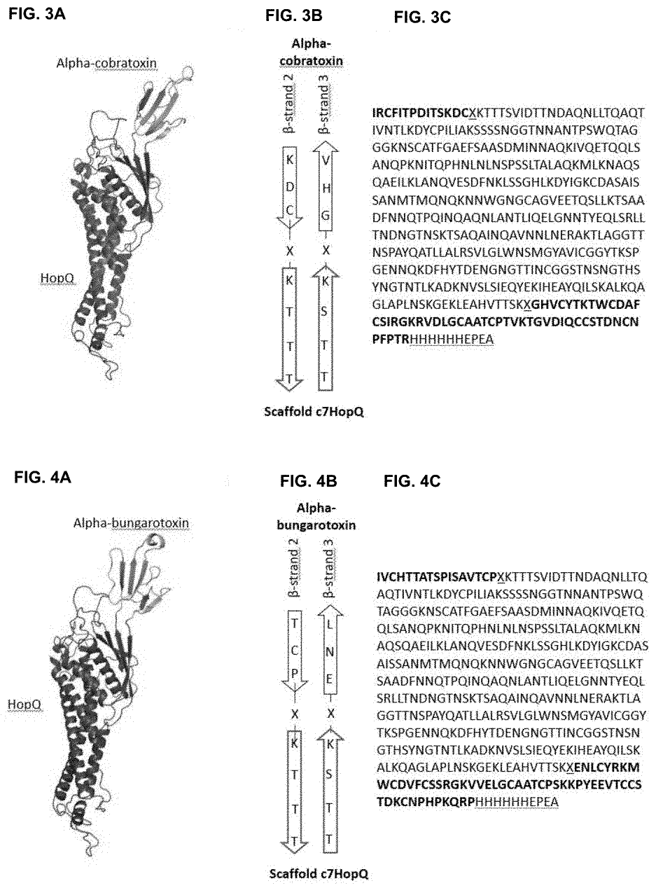

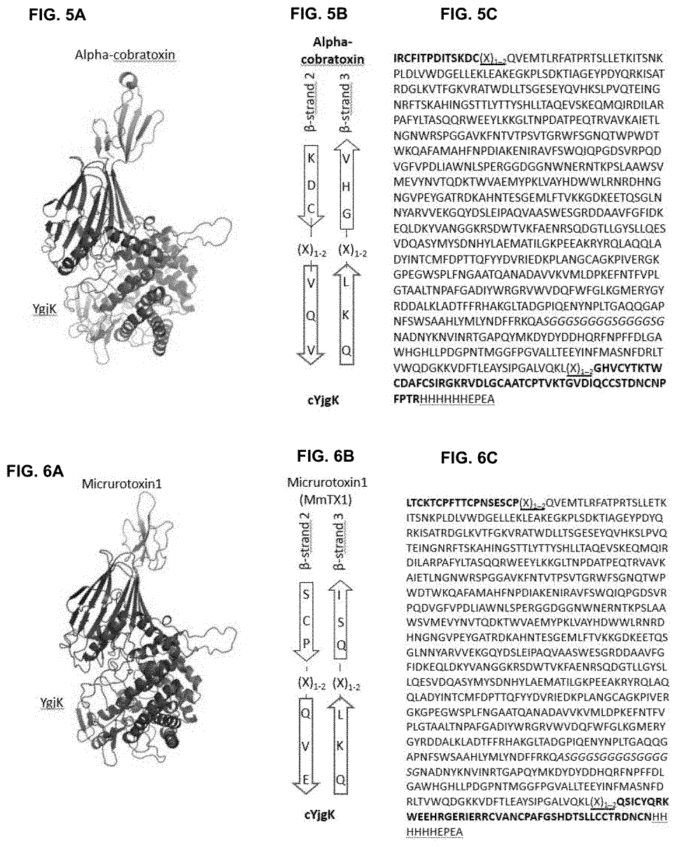

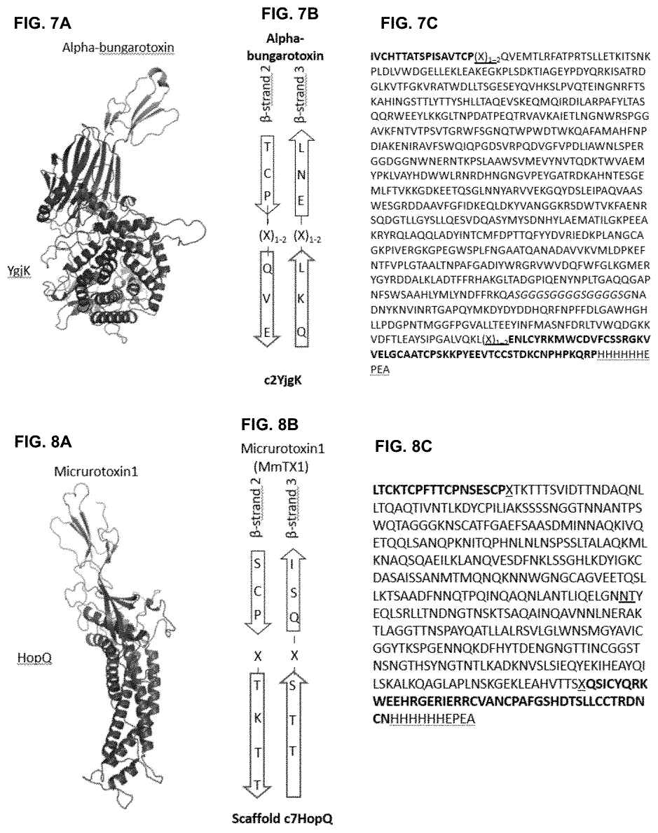

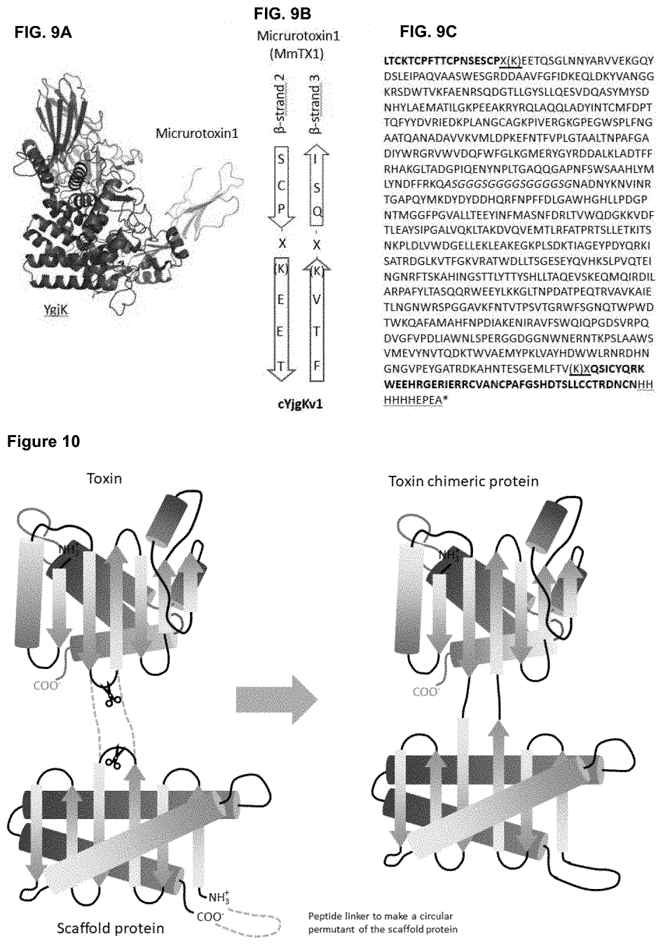

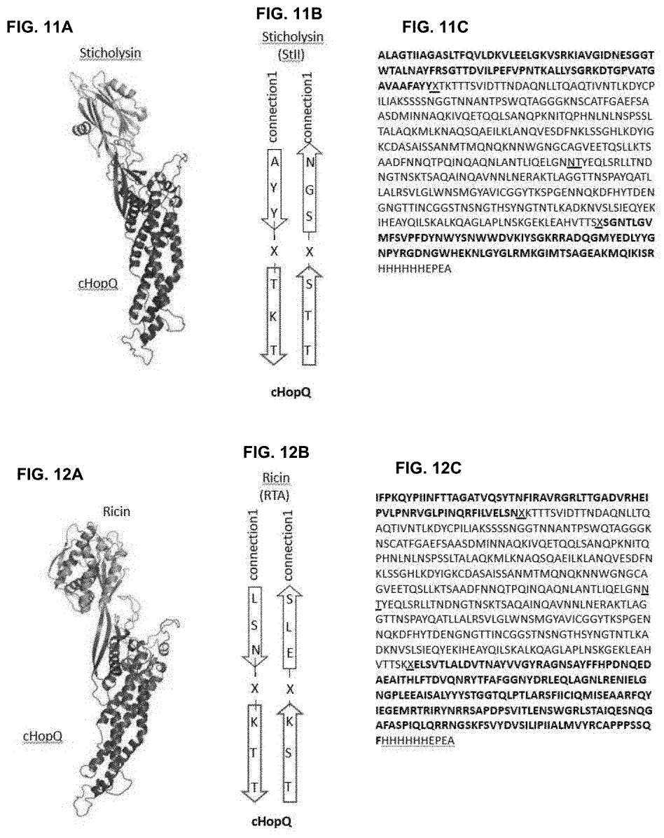

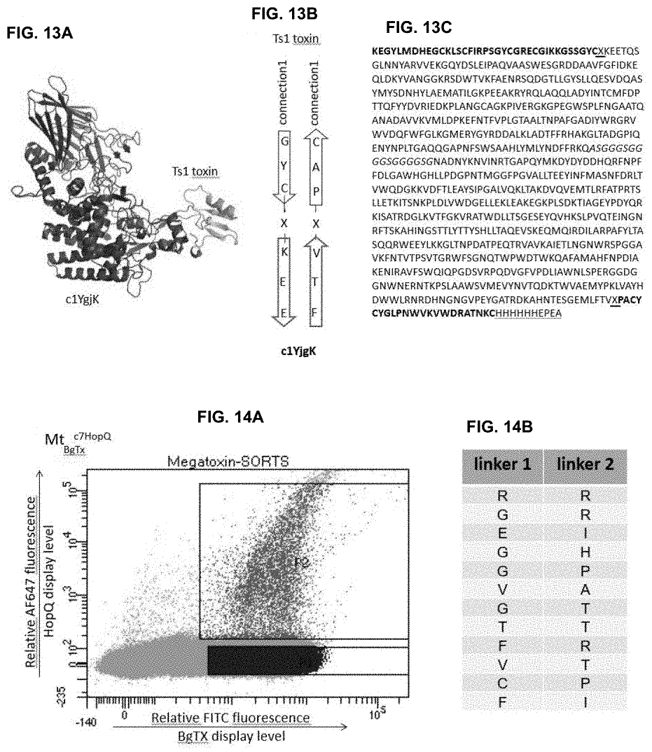

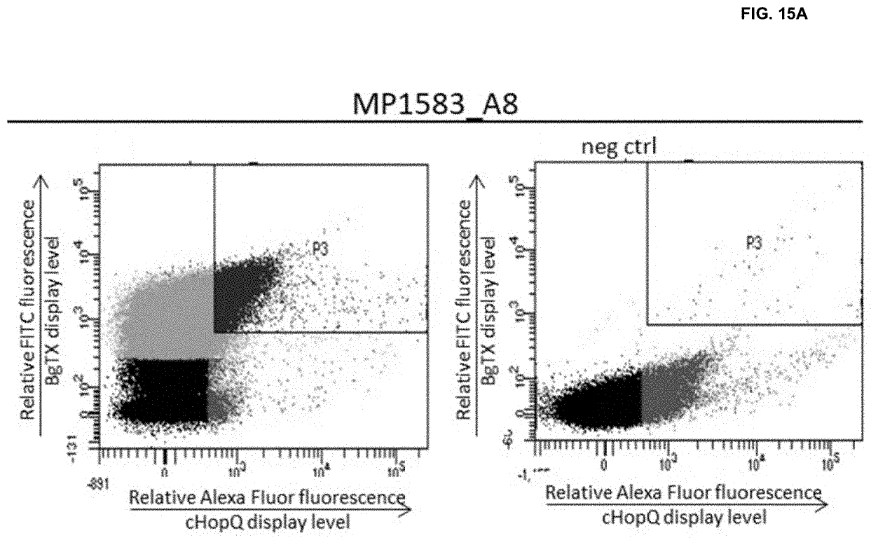

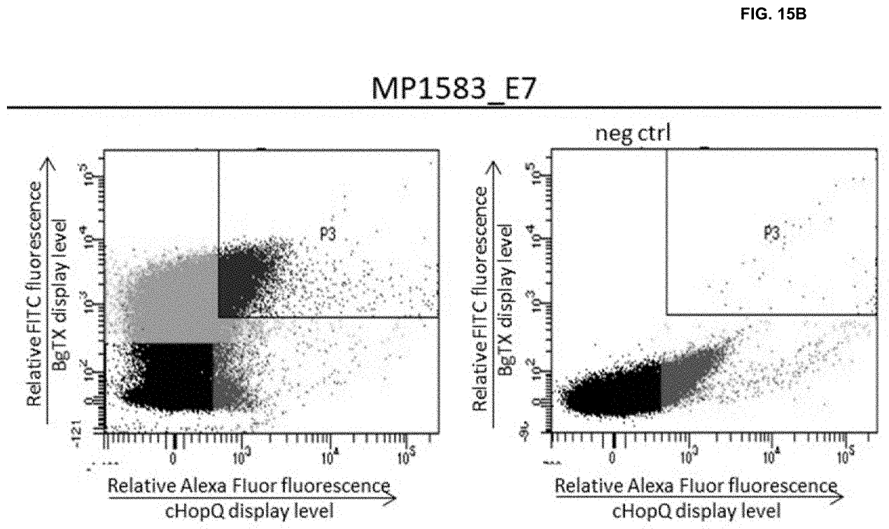

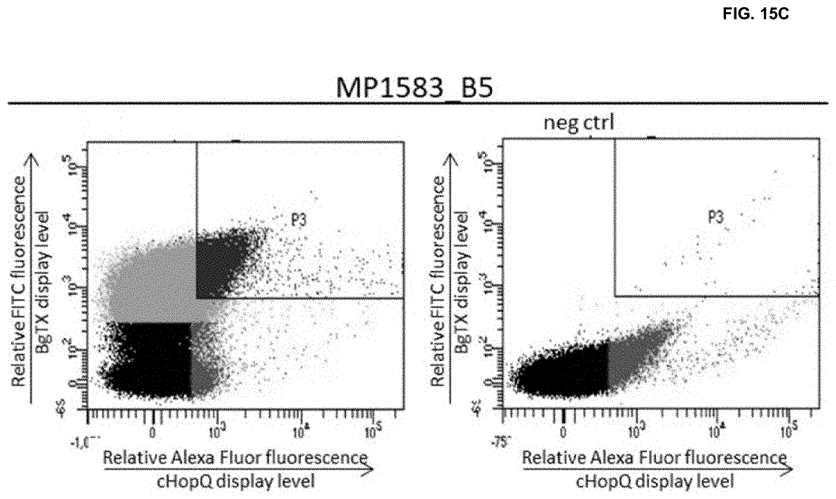

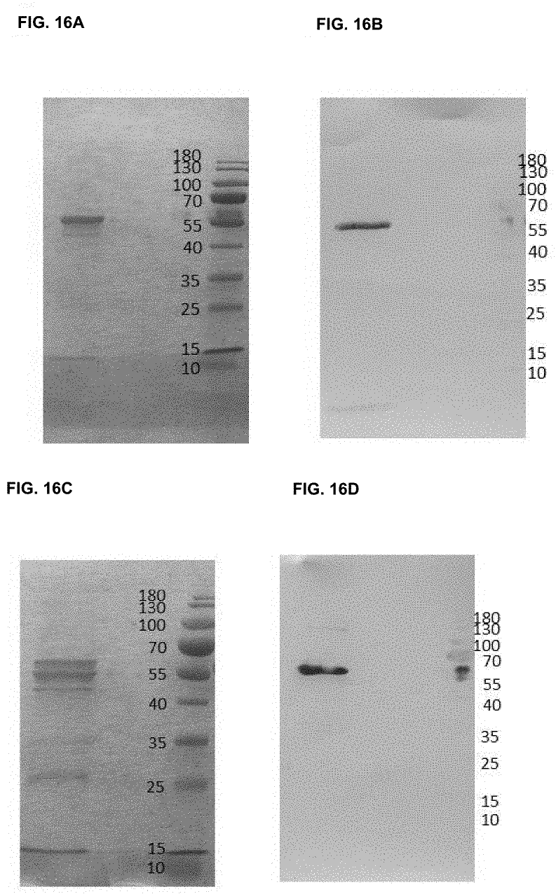

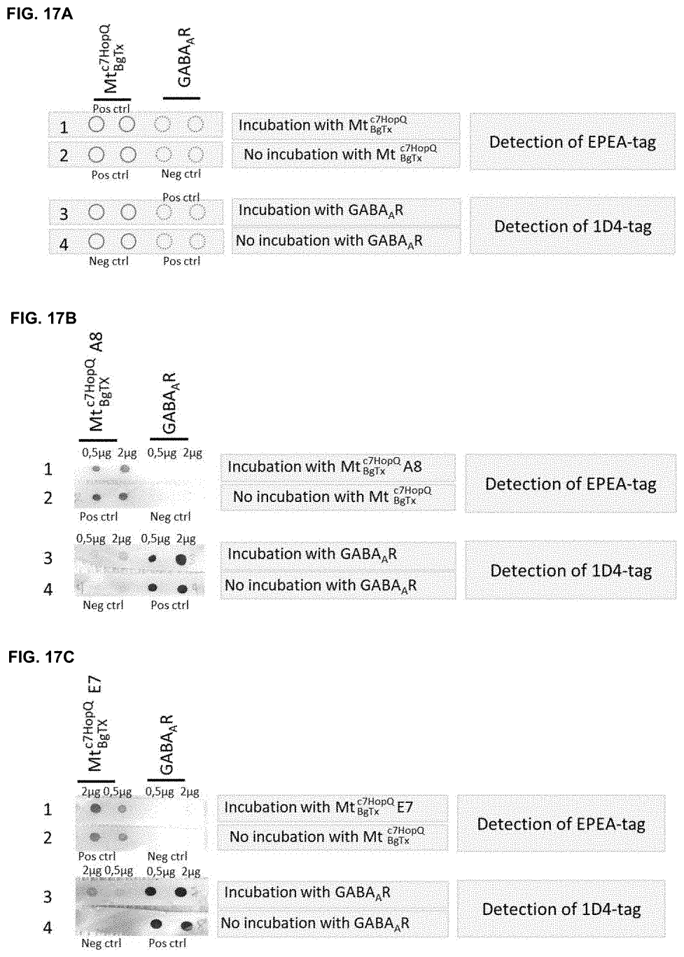

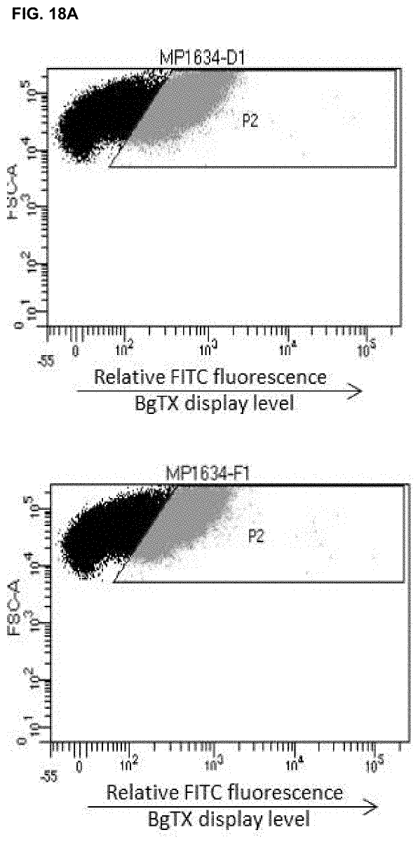

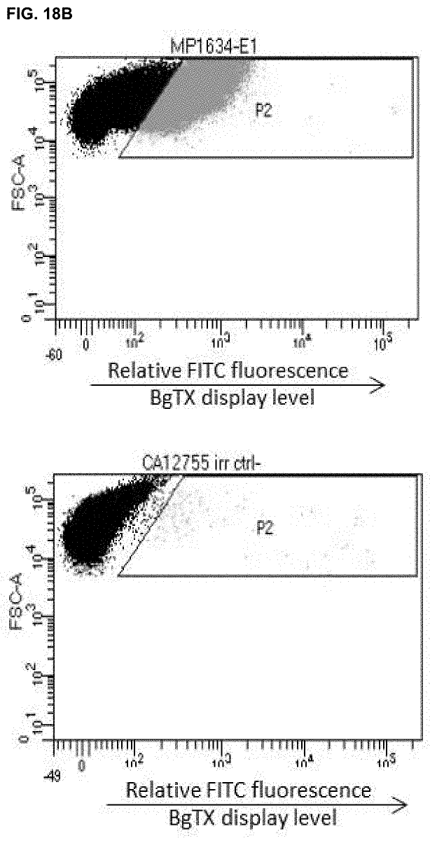

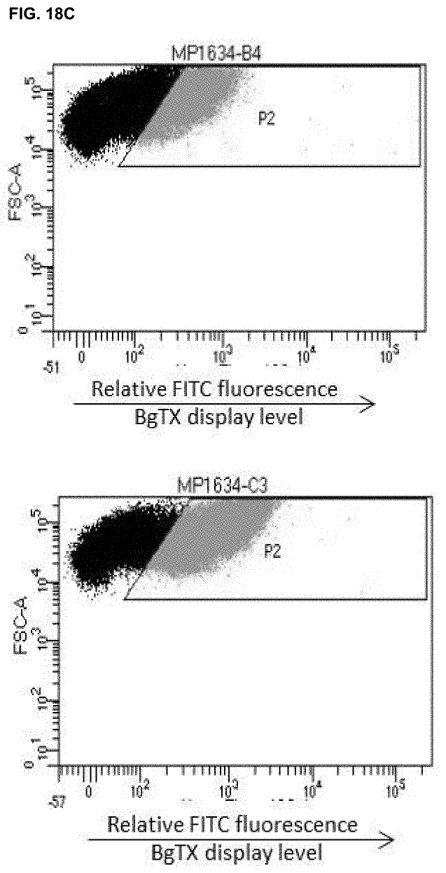

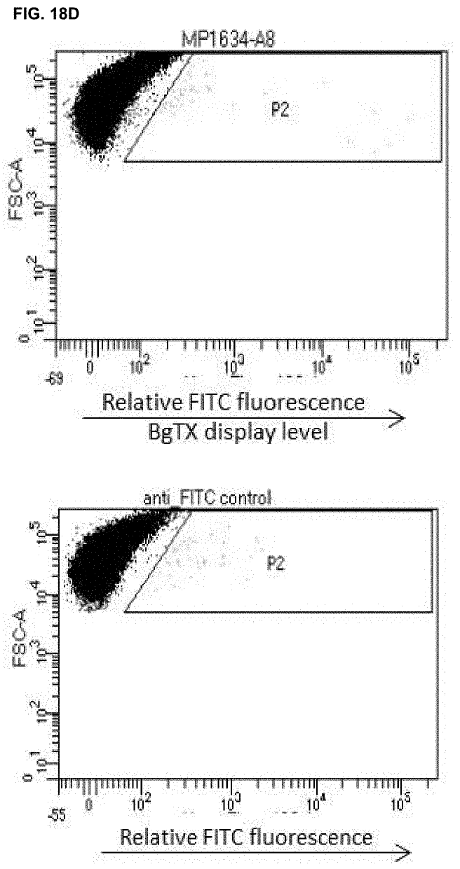

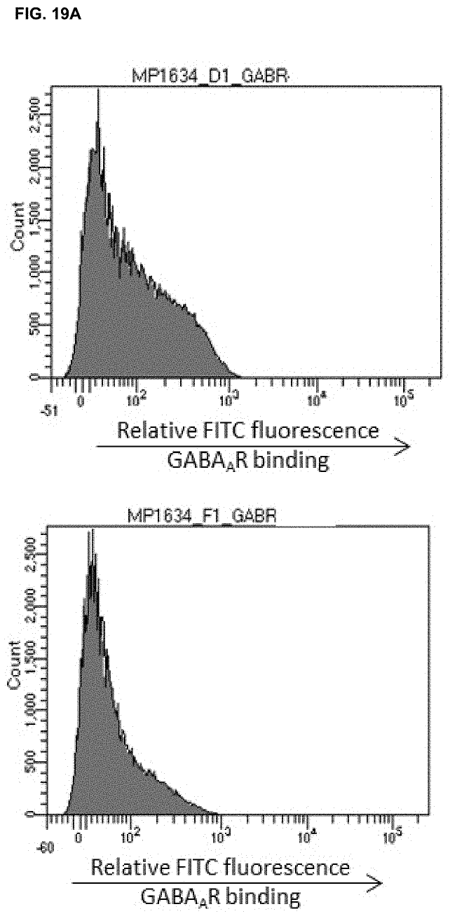

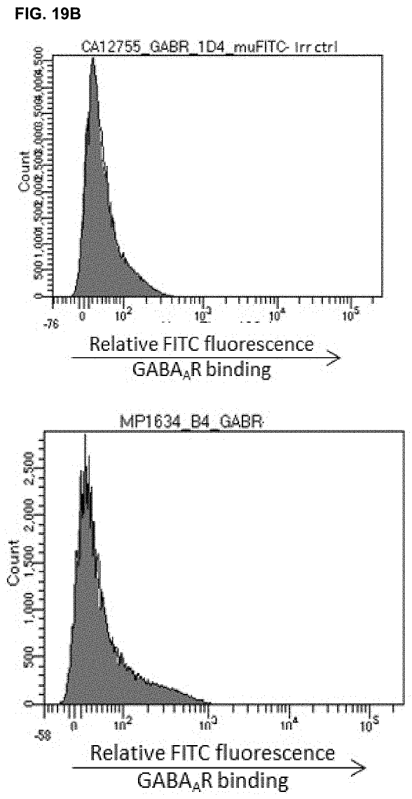

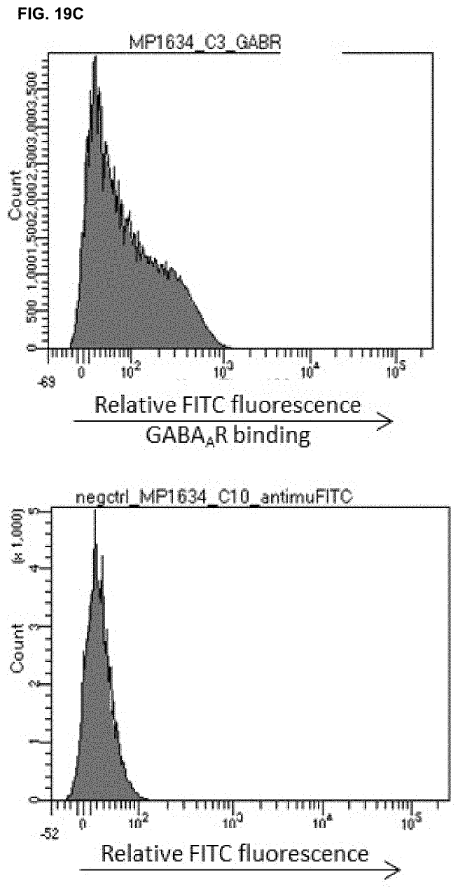

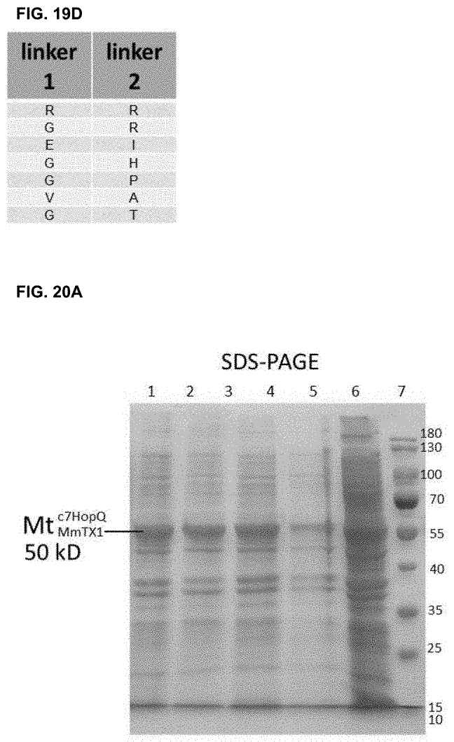

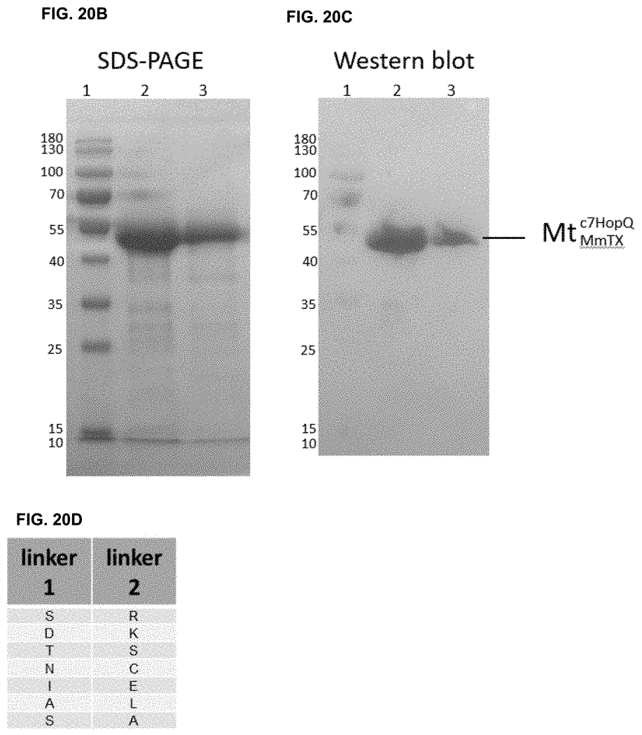

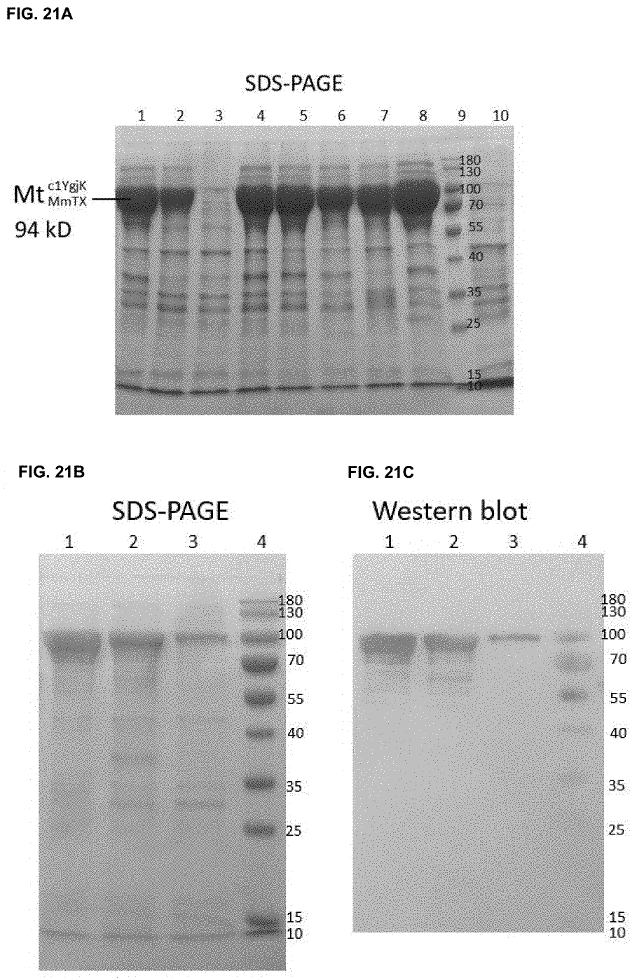

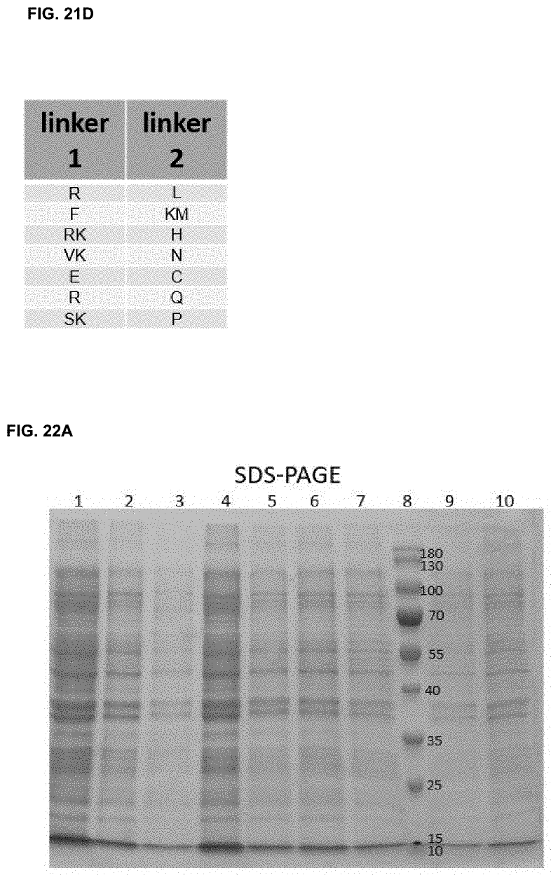

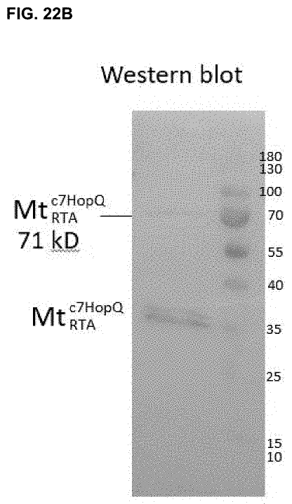

(FIG. 3A) Model of a toxin fusion protein made by fusion of alpha-cobratoxin (top) and a circularly permutated variant of the Adhesin domain of HopQ of H. pylori (bottom) via two peptide bonds or linkers that connect toxin to scaffold. (FIG. 3B) A circularly permutated gene encoding the Adhesin domain of the type 1 HopQ of Helicobacter pylori strain G27 (bottom, PDB 5LP2, SEQ ID NO:16, c7HopQ) was inserted in the β-turn of alpha-cobratoxin (top, PDB 1YI5, SEQ ID NO:1) connecting β-strand β2 to β3 (β-turn β2-β3). (FIG. 3C) Amino acid sequence of the resulting toxin fusion protein chimer (Mt_{alpha-cobratoxin}^{c7HopQ}, SEQ ID NO:2). Sequences originating from the toxin are depicted in bold. Sequences originating from HopQ are in normal text. The peptide linking the N-terminus and the C-terminus of the HopQ to make a circular permutant is depicted in italics. The C-terminal tag includes 6×His and EPEA are underlined with a dotted line.

FIGS. 4A-4C. Model of a 50 kDa alpha-bungarotoxin fusion protein built from a circularly permutated variant of HopQ inserted into the β-turn connecting β-strands β2 and β3 of the alpha-bungarotoxin.

(FIG. 4A) Model of a toxin fusion protein made by fusion of alpha-bungarotoxin (top) and a circularly permutated variant of the Adhesin domain of HopQ of H. pylori (bottom) via two peptide bonds or linkers that connect toxin to scaffold. (FIG. 4B) A circularly permutated gene encoding the Adhesin domain of the type 1 HopQ of Helicobacter pylori strain G27 (bottom, PDB 5LP2, SEQ ID NO:16, c7HopQ) was inserted in the β-turn of alpha-bungarotoxin (top, PDB 4UY2, SEQ ID NO: 3) connecting β-strand β2 to β3 (β-turn β2-β3). (FIG. 4C) Amino acid sequence of the resulting toxin fusion protein chimer (Mt_{alpha-bungarotoxin}^{c7HopQ}, SEQ ID NO:4). Sequences originating from the toxin are depicted in bold. Sequences originating from HopQ are in normal text. The C-terminal tag includes 6×His and EPEA are underlined with a dotted line.

FIGS. 5A-5C. Model of a 94 kDa alpha-cobratoxin fusion protein built from a circularly permutated variant of YgjK inserted into the β-turn connecting β-strands β2 and β3 of the alpha-cobratoxin.

(FIG. 5A) Model of a toxin fusion protein made by fusion of alpha-cobratoxin (top) and a circularly permutated variant of YgjK (bottom) via two peptide bonds or linkers that connect toxin to scaffold. (FIG. 5B) A circularly permutated gene encoding the Escherichia coli K12 YgjK (PDB 3W7S, SEQ ID NO:5) was fused so that the YgjK protein was inserted in the β-turn of alpha-cobratoxin (top, PDB 1YI5, SEQ ID NO: 1) connecting β-strand β2 to β3 (β-turn β2-β3) using short peptide linkers of variable length (1 or 2 amino acids) and random composition. (FIG. 5C) Amino acid sequence of the resulting toxin fusion proteins (Mt_{alpha-cobratoxin}^{c2YgjK}, SEQ ID NO: 6-9). Sequences originating from the toxin are depicted in bold. Sequences originating from YgjK are in normal text. X and XX are short peptide linkers of 1 AA or 2 AA and random composition. The peptide linking the N-terminus and the C-terminus of the YgjK to make a circular permutant is depicted in italics. The C-terminal tag includes 6×His and EPEA are underlined with a dotted line.

FIGS. 6A-6C. Model of a 94 kDa Micrurotoxin1 fusion protein built from a circularly permutated variant of YgjK inserted into the β-turn connecting β-strands β2 and β3 of the Micrurotoxin1.

(FIG. 6A) Model of a toxin fusion protein made by fusion of Micrurotoxin1 (MmTX1, top) and a circularly permutated variant of YgjK (bottom) via two peptide bonds or linkers that connect toxin to scaffold. (FIG. 6B) A circularly permutated gene encoding the Escherichia coli K12 YgjK (PDB 3W7S, SEQ ID NO:5) was fused so that the YgjK protein was inserted in the β-turn of Micrurotoxin1 (top, a structural homologue of bungarotoxin PDB 4UY2, SEQ ID NO: 11) connecting β-strand β2 to β3 (β-turn β2-β3) using short peptide linkers of variable length (1 or 2 amino acids) and random composition. (FIG. 6C) Amino acid sequence of the resulting toxin fusion proteins (Mt_{micrumtoxin1}^{c2YgjK}, SEQ ID NO: 12-15). Sequences originating from the toxin are depicted in bold. Sequences originating from YgjK are in normal text. The peptide linking the N-terminus and the C-terminus of the YgjK to make a circular permutant is depicted in italics. X and XX are short peptide linkers of 1 AA or 2 AA and random composition. The C-terminal tag includes 6×His and EPEA are underlined with a dotted line.

FIGS. 7A-7C. Model of a 95 kDa alpha-bungarotoxin fusion protein built from a circularly permutated variant of YgjK inserted into the β-turn connecting β-strands β2 and β3 of alpha-bungarotoxin.

(FIG. 7A) Model of a toxin fusion protein made by fusion of alpha-bungarotoxin (BgTX, top) and a circularly permutated variant of YgjK (bottom) via two peptide bonds or linkers that connect toxin to scaffold. (FIG. 7B) A circularly permutated gene encoding the E. coli K12 YgjK (PDB 3W7S, SEQ ID NO:5) was fused so that the YgjK protein was inserted in the β-turn of alpha-bungarotoxin (top, PDB 4UY2, SEQ ID NO: 3) connecting β-strand β2 to β3 (β-turn β2-β3) using short peptide linkers of variable length (1 or 2 amino acids) and random composition. (FIG. 7C) Amino acid sequence of the resulting toxin fusion proteins (Mt_{BgTX}^{c2YgjK}, SEQ ID NO: 17-20). Sequences originating from the toxin are depicted in bold. Sequences originating from YgjK are in normal text. The peptide linking the N-terminus and the C-terminus of the YgjK to make a circular permutant is depicted in italics. X and XX are short peptide linkers of 1 AA or 2 AA and random composition. The C-terminal tag includes 6×His and EPEA are underlined with a dotted line.

FIGS. 8A-8C. Model of a 50 kDa micrurotoxin1 fusion protein built from a circularly permutated variant of HopQ inserted into the β-turn connecting β-strands β2 and β3 of micrurotoxin1.

(FIG. 8A) Model of a toxin fusion protein made by fusion of micrurotoxin1 (top) and a circularly permutated variant of the Adhesin domain of HopQ of H. pylori (bottom) via two peptide bonds or linkers that connect toxin to scaffold. (FIG. 8B) A circularly permutated gene encoding the Adhesin domain of the type 1 HopQ of Helicobacter pylori strain G27 (bottom, PDB 5LP2, SEQ ID NO:16, c7HopQ) was inserted in the β-turn of micrurotoxin1 (top; a structural homologue of bungarotoxin PDB 4UY2, SEQ ID NO: 11)) connecting β-strand β2 to β3 (β-turn β2-β3). (FIG. 8C) Amino acid sequence of the resulting toxin fusion protein chimer (Mt_{MmTX1}^{c7HopQ}, SEQ ID NO: 21). Sequences originating from the toxin are depicted in bold. Sequences originating from HopQ are in normal text. The connection of the N-terminus and the C-terminus of the HopQ to make a circular permutant is double underlined The C-terminal tag includes 6×His and EPEA are underlined with a dotted line.

FIGS. 9A-9C. Model of a 94 kDa Micrurotoxin1 fusion protein built from a circularly permutated variant of YgjK inserted into the β-turn connecting β-strands β2 and β3 of the Micrurotoxin1.

(FIG. 9A) A second model of a toxin fusion protein made by fusion of Micrurotoxin1 (MmTX1, right) and a circularly permutated variant of YgjK (left) via two peptide bonds or linkers that connect toxin to scaffold. (FIG. 9B) A circularly permutated gene encoding the Escherichia coli K12 YgjK (PDB 3W7S, SEQ ID NO:5) was fused so that the YgjK protein was inserted in the β-turn of Micrurotoxin1 (a structural homologue of bungarotoxin PDB 4UY2, SEQ ID NO: 11) connecting β-strand β2 to β3 (β-turn β2-β3) using short peptide linkers of variable length (1 or 2 amino acids) and random composition. (FIG. 9C) Amino acid sequence of the resulting toxin fusion proteins (Mt_{micrurotoxin1}^{c1YgjK}, SEQ ID NO: 23-26). Sequences originating from the toxin are depicted in bold. Sequences originating from YgjK are in normal text. The peptide linking the N-terminus and the C-terminus of the YgjK to make a circular permutant is depicted in italics. X and X are short peptide linkers of 1 AA and random composition. The C-terminal tag includes 6×His and EPEA are underlined with a dotted line.

FIG. 10. Engineering principles of a toxin fusion protein built from a (circularly permutated variant of a) scaffold protein that is inserted into the β-turn connecting 2 β-strands of a toxin.

This scheme shows how a toxin can be grafted onto a large scaffold protein via two peptide bonds or two short linkers that connect the toxin to the scaffold. Scissors indicate how an exposed turn should to be cut in the toxin and in the scaffold. Dashed lines indicate how the remaining parts of the toxin and the scaffold should be concatenated by use of peptide bonds or short peptide linkers to build the toxin fusion protein.

FIGS. 11A-11C. Model of a 62 kDa sticholysin II fusion protein built from a circularly permutated variant of HopQ inserted into a β-turn connecting 2 β-strands of the sticholysin.

(FIG. 11A) Model of a toxin fusion protein made by fusion of sticholysin II (StII; top) and a circularly permutated variant of the Adhesin domain of HopQ of H. pylori (bottom) via two peptide bonds or linkers that connect toxin to scaffold. (FIG. 11B) A circularly permutated gene encoding the Adhesin domain of the type 1 HopQ of Helicobacter pylori strain G27 (bottom, PDB 5LP2, SEQ ID NO:16, c7HopQ) was inserted in a β-turn of sticholysin II (top, PDB 1072, SEQ ID NO: 27) connecting 2 β-strands. (FIG. 11C) Amino acid sequence of the resulting toxin fusion protein chimer (Mt_{StII}^{c7HopQ}, SEQ ID NO:28). Sequences originating from the toxin are depicted in bold. Sequences originating from HopQ are in normal text. The connection of the N-terminus and the C-terminus of the HopQ to make a circular permutant is double underlined. The C-terminal tag includes 6×His and EPEA are underlined with a dotted line.

FIGS. 12A-12C. Model of a 71 kDa ricin fusion protein built from a circularly permutated variant of HopQ inserted into a β-turn connecting 2 β-strands of the ricin.

(FIG. 12A) Model of a toxin fusion protein made by fusion of ricin (top) and a circularly permutated variant of the Adhesin domain of HopQ of H. pylori (bottom) via two peptide bonds or linkers that connect toxin to scaffold. (FIG. 12B) A circularly permutated gene encoding the Adhesin domain of the type 1 HopQ of Helicobacter pylori strain G27 (bottom, PDB 5LP2, SEQ ID NO:16, c7HOPQ) was inserted in a β-turn of the ricin chain A fragment 36 to 302 (top; RTA36-302, PDB 5J56, SEQ ID NO:30) connecting 2 β-strands. (FIG. 12C) Amino acid sequence of the resulting toxin fusion protein chimer (Mt_{RTA36-302}^{c7HopQ}, SEQ ID NO:31). Sequences originating from the toxin are depicted in bold. Sequences originating from HopQ are in normal text. The connection of the N-terminus and the C-terminus of the HopQ to make a circular permutant is double underlined. The C-terminal tag includes 6×His and EPEA are underlined with a dotted line.

FIGS. 13A-13C. Model of a 95 kDa Ts1 toxin fusion protein built from a circularly permutated variant of YgjK inserted into a β-turn connecting 2 β-strands of the Ts1 toxin.

(FIG. 13A) A model of a toxin fusion protein made by fusion of Ts1 toxin (Ts1; right) and a circularly permutated variant of YgjK (left) via two peptide bonds or linkers that connect toxin to scaffold. (FIG. 13B) A circularly permutated gene encoding the E. coli K12 YgjK (PDB 3W7S, SEQ ID NO:5) was fused so that the YgjK protein was inserted in a β-turn of Ts1 toxin (PDB 1B7D, SEQ ID NO: 37) connecting β-strand 2 and β-strand 3 of Ts1 toxin using short peptide linkers of random composition. (FIG. 13C) Amino acid sequence of the resulting toxin fusion proteins (Mt_{Ts1}^{c1YgjK}, SEQ ID NO: 38). Sequences originating from the toxin are depicted in bold. Sequences originating from YgjK are in normal text. The peptide linking the N-terminus and the C-terminus of the YgjK to make a circular permutant is depicted in italics. X is a short peptide linker of 1 AA and random composition. The C-terminal tag includes 6×His and EPEA are underlined with a dotted line.

FIGS. 14A and 14B. Fluorescence-activated cell sorting to select EBY100 yeast cells displaying on their surface different Mt_{BgTx}^{c7HopQ} bungarotoxin fusion proteins.

(FIG. 14A) EBY100 yeast cells transformed with pTMB2BgTx encoding toxin fusion proteins Mt_{BgTx}^{c7HopQ} with different linkers and fused to Aga2p, ACP and myc-tag (SEQ ID NO:22) were sorted using anti-bungarotoxin antibodies and anti-mouse-FITC together with an anti-HopQ labelled with alexa647. Cells that fell into the P1 gate were sorted and sequence analysed. (FIG. 14B) The amino acid sequence of the peptide linkers connecting the toxin and the scaffold protein are indicated for several variants.

FIGS. 15A-15C. Flow cytometric analysis of the display of toxin fusion protein Mt_{BgTx}^{c7HopQ} with different linker on the surface of EBY100 yeast cells.

Dot plot representations of the relative fluorescence intensity of individual EBY100 yeast cells, transformed with different pTMB2BgTx plasmids (MP1583_A8 (FIG. 15A), MP1583_E7 (FIG. 15B), MP1583_B5 (FIG. 15C)) each encoding and displaying a bungarotoxin fusion protein Mt_{BgTx}^{c7HopQ} with different linkers and fused to Aga2p and ACP (SEQ ID NO:22) are shown. The yeast cells of each clone were stained with anti-bungarotoxin and anti-rabbit-FITC to detect the presence of bungarotoxin, and compared to the same sample stained anti-HA and anti-rabbit-FITC to see the background staining.

FIGS. 16A-16D. The expression of recombinant toxin fusion proteins in E. coli cells analyzed by SDS-PAGE and Western Blot.

The Mt_{BgTx}^{c7HopQ} fusion proteins were expressed in E. coli and purified. A band with the correct size is seen on the SDS-PAGE. (FIG. 16A) Mt_{BgTx}^{c7HopQ} clone MP1583_A8 (lane 1), protein marker (PageRuler™ Prestained Protein Ladder, Fermentas cat. Nr. SM0671) (lane 2). (FIG. 16B) The presence of fusion protein was detected in Western blot by using anti-EPEA detection as explained in Example 2. (FIG. 16C) SDS-PAGE of Mt_{BgTx}^{c7HopQ} clone MP1583_E7 (lanes 1), Protein marker (PageRuler™ Prestained Protein Ladder) (lane 2). (FIG. 16D) The presence of fusion protein was detected in Western blot by using anti-EPEA detection as explained in Example 2. Mt_{BgTx}^{c7HopQ} clone MP1583_E7 (lanes 1), Protein marker (PageRuler™ Prestained Protein Ladder) (lane 2).

FIGS. 17A-17C. Binding of the Mt_{BgTx}^{c7HopQ} to GABA_{A}R 133 pentamer is confirmed by dot blot.

The Mt_{BgTx}^{c7HopQ} fusion proteins, expressed in E. coli and purified were used in a dot blot to confirm binding to the GABA_{A}R as explained in example 5. (FIG. 17A) Dot blot set-up: Mt_{BgTx}^{c7HopQ} carrying an EP EA tag was spotted onto nitrocellulose, next to the GABA_{A}R β3 carrying a 1D4-tag. Strip1 was incubated with the Mt_{BgTx}^{c7HopQ}, Strip2 was not incubated with the Mt_{BgTx}^{c7HopQ} and serves as a negative control for the binding to GABA_{A}R, and as positive control for EPEA detection. To detect binding of Mt_{BgTx}^{c7HopQ} to GABA_{A}R, strip 1 and 2 were stained by using an anti-EPEA antibody. Strip3 was incubated with the GABA_{A}R, Strip4 was not incubated with the GABA_{A}R and serves as a negative control for the binding to Mt_{BgTx}^{c7HopQ} and as positive control for the 1D4 detection. To detect binding of GABA_{A}R to Mt_{BgTx}^{c7HopQ}, strip 3 and 4 were stained by using an anti-1D4 antibody. (FIG. 17B) Mt_{BgTx}^{c7HopQ}_A8 carrying an EPEA tag was spotted onto nitrocellulose, next to the GABA_{A}R 133 pentamer. Detection of binding was done as described in A. (FIG. 17C) Mt_{BgTx}^{c7HopQ}_E7 carrying an EPEA tag was spotted onto nitrocelluse, next to the GABA_{A}R β3. Detection of binding was done as described in A.

FIGS. 18A-18D. Flow cytometric analysis of the display of a toxin fusion protein Mt_{BgTx}^{c2YgjK} with different linkers on the surface of EBY100 yeast cells.

(FIGS. 18A-18D) Dot plot representations of the relative fluorescence intensity of individual EBY100 yeast cells, transformed with different pTMB5BgTx plasmids, each encoding and displaying a toxin fusion protein Mt_{BgTx}^{c2YgjK} with different linkers and fused to Aga2p and ACP (SEQ ID NO:32-35) are shown. All samples were stained with anti-bungarotoxin and anti-rabbit-FITC to detect the presence of bungarotoxin. Yeast cells transformed with Mb_{Nb207}^{c1YgjK} (CA12755) were used as negative control for the anti-BgTX staining, Mt_{BgTx}^{c7HopQ}_E7 (anti-FITC control) was only incubated with anti-rabbit-FITC to see the FITC background staining.

FIGS. 19A-19D. Flow cytometric analysis of the binding of different toxin fusion protein Mt_{BgTx}^{c2YgjK} on the surface of EBY100 yeast cells to the GABA_{A}R 133 pentamer.

(FIGS. 19A-19C) The single-parameter histograms show the relative fluorescence intensity of different yeast clones (called MP1634_D1, F1, B4, C3), each transformed with a different pTMB5BgTx plasmid and each encoding and displaying a toxin fusion protein Mt_{BgTx}^{c2YgjK} with different linkers and fused to Aga2p and ACP (SEQ ID NO:32-35) are shown. All samples were incubated with the pentamer GABA_{A}R β3, followed by incubation with mouse anti-1D4-tag and anti-mouse-FITC to detect the binding to GABA_{A}R β3. Yeast cells transformed with Mb_{Nb207}^{c1YgjK} (CA12755) were used as negative control for the staining, MP1634_C10 (anti-mouse-FITC control) was only incubated with anti-mouse-FITC to see the FITC background staining. (FIG. 19D) Sequences of linkers connecting toxin to scaffold of individual clones expressing Mt_{BgTx}^{c2YgjK} on the surface of EBY100 yeast cells.

FIGS. 20A-20D. Expression in E. coli of toxin fusion proteins Mt_{MmTX1}^{c7HopQ}.

(FIG. 20A) The Mt_{MmTX1}^{c7HopQ} fusion proteins were expressed in E. coli. Periplasmic extracts were analysed on SDS-PAGE (lanes 1-6). Protein marker (PageRuler™ Prestained Protein Ladder) (lane 7). A band of 50 kDa corresponding to the size of Mt_{MmTX1}^{c7HopQ} was seen on the gel. (FIG. 20B) IMAC purified Mt_{MmTX1}^{c7HopQ} was analysed on an SDS-PAGE: Protein marker (PageRuler™ Prestained Protein Ladder, lane 1), Clone MP1583_C9 (lane 2), and MP1583_A8 (lane 3). (FIG. 20C) Purified Mt_{MmTX1}^{c7HopQ}, transferred to a membrane is detected in Western blot by using an anti-EPEA tag detection as explained in Example 8. The blot image showing: Protein marker (PageRuler™ Prestained Protein Ladder, lane 1), Clone MP1583_C9 (lane 2), MP1583_A8 (lane 3). A band of 50 kDa corresponding to the size of Mt_{MmTX1}^{c7HopQ} is detected. (FIG. 20D) Sequences of linkers connecting toxin to scaffold of individual clones expressing Mt_{MmTX1}^{c7HopQ} on the surface of EBY100 yeast cells.

FIGS. 21A-21D. Expression in E. coli of toxin fusion proteins Mt_{MmTX1}^{c1YgjK}.

(FIG. 21A) The Mt_{MmTX1}^{c1YgjK} fusion proteins were expressed in E. coli. Periplasmic extracts were analyzed on SDS-PAGE (lanes 1-8), Protein marker (PageRuler™ Prestained Protein Ladder, Fermentas cat. Nr. SM0671) (lane 9), and a Nb was expressed in parallel (lane10) as control. A band of 94 kDa corresponding to the size of Mt_{MmTX1}^{c1YgjK} is seen on the gel. (FIG. 21B) Mt_{MmTX1}^{c1YgjK} was analyzed on an SDS-PAGE: Clone MP1639_D3 (lane 1), MP1639_F4 (lane 2), MP1639_A9 (lane 3), protein marker (PageRuler™ Prestained Protein Ladder, lane 4). (FIG. 21C) Mt_{MmTX1}^{c1YgjK}, transferred to a membrane is detected in Western blot by using anti-EPEA tag detection as explained in Example 9. The blot image showing: Clone MP1639_D3 (lane 1), MP1639_F4 (lane 2), MP1639_A9 (lane 3), protein marker (PageRuler™ Prestained Protein Ladder, lane 4). A band of 94 kDa corresponding to the size of Mt_{MmTX1}^{c1YgjK} is detected. (FIG. 21D) Sequences of linkers connecting toxin to scaffold of individual clones expressing MtMmTX1 c1YgjK in E. coli.

FIGS. 22A-22B. Expression in E. coli of toxin fusion proteins Mt_{RTA}^{c7HopQ}.

(FIG. 22A) The Mt_{RTA}^{c7HopQ} fusion proteins were expressed in E. coli. Periplasmic extracts were analysed on SDS-PAGE (lanes 1-7, 9, 10), Protein marker (PageRuler™ Prestained Protein Ladder) (lane 8). No specific band corresponding to the size of Mt_{R-m}^{c7HopQ} was visible on the gel. (FIG. 22B) Affinity purified Mt_{R-m}^{c7HopQ} was loaded on SDS-PAGE and transferred to a membrane. Detection of Mt_{RTA}^{c7HopQ} in Western blot is done by an anti-EPEA tag detection as explained in Example 11. The blot image showing: purified Mt_{RTA}^{c7HopQ} (lane 1), Protein marker (lane 2). A very faint band of 71 kDa corresponding to the size of Mt_{MmTX1}^{c7HopQ} is detected, next to smaller bands around 35 kDa indicating that Mt_{R-m}^{c7HopQ} fusion protein is cleaved.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

With a “genetic construct”, “chimeric gene”, “chimeric construct” or “chimeric gene construct” is meant a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence. The regulatory nucleic acid sequence of the chimeric gene is not operatively linked to the associated nucleic acid sequence as found in nature. In particular, the term “genetic fusion construct” as used herein refers to the genetic construct encoding the mRNA that is translated to the fusion protein of the invention as disclosed herein.

The term “vector”, “vector construct,” “expression vector,” or “gene transfer vector,” as used herein, is intended to refer to a nucleic acid molecule capable of transporting another nucleic acid molecule to which it has been linked, and includes any vector known to the skilled person, including any suitable type including, but not limited to, plasmid vectors, cosmid vectors, phage vectors, such as lambda phage, viral vectors, such as adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Expression vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Suitable vectors have regulatory sequences, such as promoters, enhancers, terminator sequences, and the like as desired and according to a particular host organism (e.g. bacterial cell, yeast cell). Cloning vectors are generally used to engineer and amplify a certain desired DNA fragment and may lack functional sequences needed for expression of the desired DNA fragments. The construction of expression vectors for use in transfecting prokaryotic cells is also well known in the art, and thus can be accomplished via standard techniques (see, for example, Sambrook, et al. Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. ‘Host cells’ can be either prokaryotic or eukaryotic. The cells can be transiently or stably transfected.

Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. For all standard techniques see, for example, Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016). Recombinant host cells, in the present context, are those which have been genetically modified to contain an isolated DNA molecule, nucleic acid molecule or expression construct or vector of the invention. The DNA can be introduced by any means known to the art which are appropriate for the particular type of cell, including without limitation, transformation, lipofection, electroporation or viral mediated transduction. A DNA construct capable of enabling the expression of the chimeric protein of the invention can be easily prepared by the art-known techniques such as cloning, hybridization screening and Polymerase Chain Reaction (PCR). Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (2012), Wu (ed.) (1993) and Ausubel et al. (2016). Representative host cells that may be used with the invention include, but are not limited to, bacterial cells, yeast cells, plant cells and animal cells. Bacterial host cells suitable for use with the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Animal host cells suitable for use with the invention include insect cells and mammalian cells (most particularly derived from Chinese hamster (e.g. CHO), and human cell lines, such as HeLa. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. The host cells may be provided in suspension or flask cultures, tissue cultures, organ cultures and the like. Alternatively, the host cells may also be transgenic animals.

The terms “protein”, “polypeptide”, “peptide”, or “small protein” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). The term “peptide” or “small protein” may be limited in the number of amino acids typically not more than about 40, 50, 60, 70, 80, 90, or 100 residues. By “recombinant polypeptide” is meant a polypeptide made using recombinant techniques, i.e., through the expression of a recombinant or synthetic polynucleotide. When the chimeric polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation. By “isolated” is meant material that is substantially or essentially free from components that normally accompany it in its native state. For example, an “isolated polypeptide” refers to a polypeptide which has been purified from the molecules which flank it in a naturally-occurring state, e.g., a fusion protein as disclosed herein which has been removed from the molecules present in the production host that are adjacent to said polypeptide. An isolated chimer can be generated by amino acid chemical synthesis or can be generated by recombinant production. The expression “heterologous protein” may mean that the protein is not derived from the same species or strain that is used to display or express the protein.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant”, “analogue” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product. Alternatively, a variant may also include synthetic molecules, e.g. a toxin ligand variant may be similar in structure and/or function to the natural toxin, but may concern a small molecule, or a synthetic peptide or protein, which is man-made.

A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions. Protein secondary structure elements (SSEs) typically spontaneously form as an intermediate before the protein folds into its three dimensional tertiary structure. The two most common secondary structural elements of proteins are alpha helices and beta (β) sheets, though β-turns and omega loops occur as well. Beta sheets consist of beta strands (also β-strand) connected laterally by at least two or three back-bone hydrogen bonds, forming a generally twisted, pleated sheet. A β-strand is a stretch of poly-peptide chain typically 3 to 10 amino acids long with backbone in an extended conformation. AB-turn is a type of non-regular secondary structure in proteins that causes a change in direction of the polypeptide chain. Beta turns (β turns, β-turns, β-bends, tight turns, reverse turns) are very common motifs in proteins and polypeptides, which mainly serve to connect β-strands.

The term “circular permutation of a protein” or “circularly permutated protein” refers to a protein which has a changed order of amino acids in its amino acid sequence, as compared to the wild type protein sequence, with as a result a protein structure with different connectivity, but overall similar three-dimensional (3D) shape. A circular permutation of a protein is analogous to the mathematical notion of a cyclic permutation, in the sense that the sequence of the first portion of the wild type protein (adjacent to the N-terminus) is related to the sequence of the second portion of the resulting circularly permutated protein (near its C-terminus), as described for instance in Bliven and Prlic (2012). A circular permutation of a protein as compared to its wild protein is obtained through genetic or artificial engineering of the protein sequence, whereby the N- and C-terminus of the wild type protein are ‘connected’ and the protein sequence is interrupted at another site, to create a novel N- and C-terminus of said protein. The circularly permutated scaffold proteins of the invention are the result of a connected N- and C-terminus of the wild type protein sequence, and a cleavage or interrupted sequence at an accessible or exposed site (preferentially a β-turn or loop) of said scaffold protein, whereby the folding of the circularly permutate scaffold protein is retained or similar as compared to the folding of the wild type protein. Said connection of the N- and C-terminus in said circularly permutated scaffold protein may be the result of a peptide bond linkage, or of introducing a peptide linker, or of a deletion of a peptide stretch near the original N- and C-terminus if the wild type protein, followed by a peptide bond or the remaining amino acids.

The term “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link. The terms “chimeric polypeptide”, “chimeric protein”, “chimer”, “fusion peptide”, “fusion protein”, or “non-naturally-occurring protein” are used interchangeably herein and refer to a protein that comprises at least two separate and distinct polypeptide components that may or may not originate from the same protein. The term also refers to a non-naturally occurring molecule which means that it is man-made. The term “fused to”, and other grammatical equivalents, such as “covalently linked”, “connected”, “attached”, “ligated”, “conjugated” when referring to a chimeric polypeptide (as defined herein) refers to any chemical or recombinant mechanism for linking two or more polypeptide components. The fusion of the two or more polypeptide components may be a direct fusion of the sequences or it may be an indirect fusion, e.g. with intervening amino acid sequences or linker sequences, or chemical linkers. The fusion of two polypeptides or of a toxin and a scaffold protein, as described herein, may also refer to a non-covalent fusion obtained by chemical linking. For instance, the C-terminus of the β2 β-strand and the N-terminus of the β3 β-strand of the venom toxin core domain could both be linked to a chemical unit, which is capable of binding a complementary chemical unit or binding pocket linked or fused to parts or full length (circularly permutated) scaffold protein, at its exposed or accessible sites.

As used herein, the term “protein complex” or “complex” refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions. A protein complex can be a non-covalent interaction of only proteins, and is then referred to as a protein-protein complex; for instance, a non-covalent interaction of two proteins, of three proteins, of four proteins, etc. More specifically, a complex of the fusion protein and the toxin target, or a complex of the toxin and the toxin target specifically binding to the toxin. The protein complex of the functional fusion protein, bound by its toxin part to a target, for which said target is known to bind to specifically bind said toxin, will be the complex formed that is used herein. For instance, it is used in 3D structural analysis, wherein it is the aim to resolve the structure of and interaction between the toxin target, such as the receptor or ion channel or transporter, and the toxin that is part of the fusion protein. It is less relevant whether the full structure of the fusion protein is determined. It will be understood that a protein complex can be multimeric.

As used herein, the terms “determining,” “measuring,” “assessing,” and “assaying” are used interchangeably and include both quantitative and qualitative determinations.

The terms “suitable conditions” refers to the environmental factors, such as temperature, movement, other components, and/or “buffer condition(s)” among others, wherein “buffer conditions” refers specifically to the composition of the solution in which the assay is performed. The said composition includes buffered solutions and/or solutes such as pH buffering substances, water, saline, physiological salt solutions, glycerol, preservatives, etc. for which a person skilled in the art is aware of the suitability to obtain optimal assay performance.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. In general, a binding domain can be immunoglobulin-based or immunoglobulin-like or it can be based on domains present in proteins, including but not limited to microbial proteins, protease inhibitors, toxins, fibronectin, lipocalins, single chain antiparallel coiled coil proteins or repeat motif proteins. Binding also includes the interaction between a ligand and its receptor, or also include the toxin and toxin target interactions. By the term “specifically binds,” as used herein is meant a binding domain which recognizes a specific target, but does not substantially recognize or bind other molecules in a sample. For a toxin, it is known to be a high affinity binder for specifically binding a toxin target, which can be a receptor, an ion channel, a transporter, among others, so the binding to its target is specific. Though specific binding does not mean exclusive binding. However, specific binding does mean that such toxins or vice versa such targets, have a certain increased affinity or preference for one or a few toxin family members or vice versa target family members. The term “affinity”, as used herein, generally refers to the degree to which a ligand (as defined further herein) binds to a target protein so as to shift the equilibrium of target protein and ligand toward the presence of a complex formed by their binding. Thus, for example, where a receptor and a ligand are combined in relatively equal concentration, a ligand of high affinity will bind to the receptor so as to shift the equilibrium toward high concentration of the resulting complex.

Methods of determining the spatial conformation of amino acids are known in the art, and include, for example, X-ray crystallography and multi-dimensional nuclear magnetic resonance. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

Finally, the term “functional fusion protein” or “conformation-selective fusion protein” in the context of the present invention refers to a fusion protein that is functional in binding to its toxin target protein, optionally in a conformation-selective manner, and in activation/inactivation of the target (depending on the known features of the toxin). A binding domain that selectively binds to a particular conformation of a target protein refers to a binding domain that binds with a higher affinity to a target in a subset of conformations than to other conformations that the target may assume. One of skill in the art will recognize that binding domains that selectively bind to a particular conformation of a target will stabilize or retain the target in this particular conformation. For example, an active state conformation-selective binding domain will preferentially bind to a target in an active conformational state and will not or to a lesser degree bind to a target in an inactive conformational state, and will thus have a higher affinity for said active conformational state; or vice versa. The terms “specifically bind”, “selectively bind”, “preferentially bind”, and grammatical equivalents thereof, are used interchangeably herein. The terms “conformational specific” or “conformational selective” are also used interchangeably herein, and all provide for functionalities of said fusion protein.

### DETAILED DESCRIPTION

The present application relates to the design and generation of novel functional fusion proteins and uses thereof, such as their role as next generation chaperones in structural analysis, or as a therapeutic. The fusion proteins as described herein are based on the finding that toxin proteins or peptides can be enlarged into rigid fusion proteins to facilitate the structural analysis of target-bound complexes in certain conformational states. Depending on the type of scaffold protein where the toxin is fused with, therapeutic application may as well be envisaged for said functional fusion proteins. In fact, the disclosure provides for a fusion protein based on the given that families or even superfamilies of toxins share sequence similarity and more importantly exhibit structural homology, although they do not exhibit functional similarity. Since toxins are grouped according to their function and/or their structure, one can start from the similarities in structural elements within a subgroup of toxins to design the generic fusion scheme. For instance, for one family with a homologous tertiary structure, the position in the structural domain that is exposed and accessible for fusion with a scaffold protein can be generally applied, taking into account the position of its target binding site, which should be avoided, resulting in the formation of a toxin-integrated fusion protein acting as chaperone for structural analysis of toxin/target complexes. The presented fusion proteins thereby provide a novel tool to facilitate high-resolution cryo-EM and X-ray crystallography structural analysis of toxin/target complexes by adding mass and supplying structural features. So the design and generation of these next-generation chaperones will allow for structural analysis of any possible complex of fusions including toxin peptides or variants thereof with their target thereby adding mass and structurally defined features to the complex of interest to obtain high resolution structures without altering conformational states. In fact, the functional fusion proteins are therefore advantageous as a tool in structural and pharmacological analysis, but also in structure-based drug design and screening, and become an added value for discovery and development of novel biologicals and small molecule agents. Finally, their potential as a therapeutic agent may be envisaged herein, as the enlarged toxins may overcome several drawbacks that have been observed for protein toxin-based drugs, such as an improved manufacturability and half-life can be expected when suitable scaffold proteins are applied to generate the functional fusions.

A novel concept for the design of rigidly fused toxin-containing fusion proteins is presented herein. The novel fusion proteins originate through generation of fusions between a toxin and a scaffold protein, wherein the scaffold protein interrupts the topology of the toxin protein or peptide, which surprisingly still appears in its typical fold and functions to specifically bind its cognate target, in a similar manner as compared to the non-fused toxin protein or peptide. The novel fusion proteins are demonstrated herein as fusions originating from three-finger fold toxins, through an interruption of the toxin domain amino acid sequence allowing insertion of a scaffold protein, thereby interrupting the topology of the toxin protein, which still appears in its typical fold and functions to specifically bind its target, in a similar manner as compared to the non-fused toxin. A classical junction of polypeptide components, while typically unjoined in their native state, is performed by joining their respective amino (N-) and carboxyl (C-) termini directly or through a peptide linkage to form a single continuous polypeptide. These fusions are often made via flexible linkers, or at least connected in a flexible manner, which means that the fusion partners are not in a stable position or conformation with respect to each other. As presented in FIG. 1A, by linking proteins via the N- and C-terminal ends, a simple linear concatenation, the fusion is easy, but may be non-stable, prone to degradation, and in some case therefore resulting in non-functional ligand protein. On the other hand, a rigid chimeric/fusion protein as presented herein, with one or more fusion points or connections within the primary topology of two or more proteins, possesses at least one non-flexible fusion point (FIG. 1B). The invention inherently comprises a toxin protein or peptide wherein rotation or bending of the toxin protein opposed to its fusion partner, the folded scaffold protein, is prohibited via the creation of several fusions. Through the presence of several fusions within the same chimer, an improved rigidity of the novel chimer of the invention is obtained, and is the result of perfectly designing the fusion sites to allow a fusion that can still retain its toxin domain fold, as well as its function to bind its target. The rigidity of a protein is in fact inherent to the (tertiary) structure of the protein, in this case the novel chimera. It has been shown that increased rigidity can be obtained by altering topologies of known protein folds (King et al., 2015). The rigidity of the fusion created in the fusion protein of the invention hence provides for a rigidity sufficiently strong to ‘orient’ or ‘fix’ the toxin receptor where the fused toxin specifically binds to, though mostly the rigidity will still be lower than the rigidity of the target itself. This interruption of primary topology, but not final tertiary structure of the toxin fold, does not affect target binding, leading to functionality and the opening of therapeutically relevant avenues in the fields involving toxin structural biology and drug discovery. The present invention relates to a novel combination of providing unique next-generation fusion technology, and high affinity and/or conformation-selective toxin target-binding potential, to allow non-covalent binding of proteins. This novel type of functional fusion proteins aids in several valuable applications depending on the type of toxin or toxin variant, or the type of folded scaffold protein that is used for the generation of the fusion protein. The advantages are numerous, with a straightforward use in structural biology, to facilitate Cryo-EM and X-ray crystallography, by adding mass to the toxin ligand, and further improving these toxins as pharmacological tools in small molecule drug design. Depending on the toxin or its target of interest, further applications of the fusion proteins of the invention are found to specifically involve druggable target sites to enable screening for pathway-selective highly potent compounds. With the rapid advancement of such technologies in biotechnology, it is foreseeable that the invention will impact the creation of novel protein therapeutics and in improved performance of current protein drugs.

Protein toxins are produced by many species, such as for instance the Ricin toxin (also see Example 11), which originates from Ricinus communis or castor bean plants, and is a heterodimer consisting of RTA, a ribosome-inactivating protein, and RTB, a lectin that facilitates receptor-mediated uptake into mammalian cells. Venom toxins concern the poison produced by some snakes, scorpions, as mentioned herein, transmitted by biting or stinging. So venom is any poisonous compound secreted by an animal intended to harm or disable another. When an organism produces a venom, its final form may contain hundreds of different bioactive elements, such as peptides, proteins and non-proteins small molecules, that interact with each other inevitably producing its toxic effects. The active components of these venoms are isolated, purified, and screened in assays. These may be either phenotypic assays to identify component that may have desirable therapeutic properties (forward pharmacology) or target directed assays to identify their biological target and mechanism of action (reverse pharmacology). In this way, toxic venomous poisons may be a starting point for a therapeutic drug. Venom in medicine is the medicinal use of venoms for therapeutic benefit in treating diseases. The term ‘venom toxin’ is defined herein as the peptidic toxins that are produced and secreted in venom of animals of the genus Conus (cone snails), arthropods (spiders, scorpions, centipedes, bees, etc.), vertebrates (snakes, lizards, etc.), and cnidarians (jellyfishes, sea anemones, etc.), insects, and worms. For an overview of those toxins and their targets, see the Venomzone platform (https://venomzone.expasy.org/). Venom toxins produced by these different organisms contain peptides that have evolved to have highly selective and potent pharmacological effects on specific targets for protection and predation. Several toxin-derived peptides have become drugs and are used for the management of diabetes, hypertension, chronic pain, and other medical conditions. Despite the similarity in their composition, toxin-derived peptide drugs have very profound differences in their structure and conformation, in their physicochemical properties (that affect solubility, stability, etc.), and subsequently in their pharmacokinetics (the processes of absorption, distribution, metabolism, and elimination following their administration to patients) (also see Stepensky 2018). In the scope of the invention, it is important to align the conserved structural regions within a venom toxin family in order to find the suitable ‘generically applicable’ manner of designing the fusion protein according to the invention.

Non-limiting examples described herein relate to Sticholysin II (StnII) (also see Example 10), which is a 20 kDa protein from the sea-anemone Stichodactyla helianthus which shows a cytotoxic activity by forming oligomeric aqueous pores in the cell plasma membrane. Sticholysin II binds specifically to sphingomyelin by two domains that recognize respectively the hydrophilic (i.e. phosphorylcholine) and the hydrophobic (i.e. ceramide) moieties of the molecule. Another non-limiting example disclosed herein is the anti-mammalian β-toxin Ts1 (see also Example 12), the main component of the Brazilian scorpion Tityus serrulatus venom, a neurotoxin that has upon recombinant production been shown to block Na⁺ current through NaV1.5 channels without affecting the processes of activation and inactivation. The folding of the polypeptide chain of Ts1 is similar to that of other scorpion toxins. A cysteine-stabilised alpha-helix/beta-sheet motif forms the core of the flattened molecule. All residues identified as functionally important by chemical modification and site-directed mutagenesis are located on one side of the molecule, which is therefore considered as the Na⁺ channel recognition site. For the purpose of the functional fusion proteins of the present invention, the skilled person should use the structural basis available in the public domain for such a toxin, in combination with the state of the art functional data to determine the exposed β-turns that will be suitable for fusing the toxin with the scaffold protein without losing the target binding or toxin functionality in the final fusion protein.

Another non-limiting example disclosed herein provides for snake venoms, which are complex mixtures of pharmacologically active peptides and protein toxins, belonging to a small number of super families of proteins. One of those super families involve three-finger fold toxins, which form a superfamily of non-enzymatic proteins found in all families of snakes.

Three-finger fold toxins have a common structure of three β-stranded loops comprising a number of β-strands extending from or forming a central core containing all four conserved disulphide bonds. Despite the common scaffold, they bind to different receptors/acceptors and exhibit a wide variety of biological effects. Thus, the structure-function relationships of this group of toxins are complicated and challenging. Studies have shown that the functional sites in these ‘sibling’ toxins are located on various segments of the molecular surface. Targeting to a wide variety of receptors and ion channels and hence distinct functions in this group of mini proteins is achieved through a combination of accelerated rate of exchange of segments as well as point mutations in exons (Kini and Doley, 2010).

All three-finger fold toxins have structurally conserved regions which contribute to the proper folding and structural integrity of the polypeptide chain. In addition to eight conserved cysteine residues found in the core region, which allow forming up to five disulfide bridges, four of which are conserved within the entire group in the central core, they also have a conserved aromatic residue (often Tyr25 or Phe27) needed for the stabilization of the β-sheet and the correct folding of the protein. Some charged amino acid residues (e.g., Asp60 in α-cobratoxin) have also been conserved and they stabilize the native conformation of the protein by forming a salt link with the C or N-terminus of the toxin. In general, they are monomers and have a short N- and C-terminal two residues before and after the first and the last cysteine residues respectively. Most three-finger fold toxins have minor differences in their loop length and conformation, particularly with homologous turns and twists. The structure is essentially flat with a small concavity. The folding pattern can slightly change between toxins depending on small variations in the size and turns of the loops, or in the number of strands. The functional sites are located on the C-tail and/or the surface of the loops, but there's no specific or common location for all of them.

Three finger-fold toxins are classified according to their biological effects as neurotoxins (α-neurotoxins, inhibitors of the muscle nicotinic acetylcholine receptors; κ-bungarotoxins, that selectively target neuronal nicotinic acetylcholine receptors; and muscarinic toxins, agonists or antagonists of muscarinic acetylcholine receptors), inhibitors of the acetylcholinesterase (fasciculins), cardiotoxins (cytotoxins that form pores in the membranes), β-cardiotoxins and related toxins (bind to β1 and β2 adrenergic receptors), nonconventional toxins (candoxins), L-type calcium channel blockers (calciseptines), platelet aggregation inhibitors (dendroaspins, antagonists of cell-adhesion processes) and other three-finger fold toxins.

In a particular example, α-Cobratoxin (also see Examples 1 and 3) was used to demonstrate the fusion protein design as described further herein. α-Cobratoxins are part of the three-finger fold superfamily and form three hairpin type loops with its polypeptide chain. The two minor loops are loop I (amino acids 1-17) and loop III (amino acids 43-57). Loop II (amino acids 18-42) is the major one. Following these loops, α-cobratoxin has a tail (amino acids 58-71). The loops are knotted together by four disulfide bonds (Cys3-Cys20, Cys14-Cys41, Cys45-Cys56, and Cys57-Cys62). Loop II contains another disulfide bridge at the lower tip (Cys26-Cys30). Stabilization of the major loop occurs through β-sheet formation. The β-sheet structure extends to amino acids 53-57 of loop III. Here it forms a triple-stranded, antiparallel β-sheet. This g-sheet has an overall right-handed twist. This β-sheet consists of eight hydrogen bonds. The folded tip is held stable by two α-helical and two β-turn hydrogen bonds. The first loop is stabilized because of one β-turn and two β-sheet hydrogen bonds. Loop III stays intact because of a β-turn and hydrophobic interactions. The tail of the α-cobratoxin structure is attached to the rest of the structure by disulfide bridge Cys57-Cys62. It is also stabilized by the tightly hydrogen bound side chain of Asn63. α-Cobratoxin can occur in both a monomeric form and a disulfide-bound dimeric form. α-Cobratoxin dimers can be homodimeric as well as heterodimeric with cytotoxin 1, cytotoxin 2 and cytotoxin 3. As a homodimer it is still able to bind to muscle type and α7 nAChR nicotinic acetylcholine receptors, but with a lower affinity than in its monomeric form. In addition, the homodimer acquires the capacity to block α-3/β-2 nACh Rs.

In a first aspect, the invention relates to a functional fusion protein comprising a toxin protein, such as a venom toxin, fused with a scaffold protein, which is a folded protein of at least 50 amino acids, wherein said toxin contains a domain with at least 3 β-strands, also referred to herein as a β-strand-containing domain, as is the case for instance for a three-finger fold toxin, wherein said scaffold protein interrupts the topology of the toxin domain at one or more accessible sites in an exposed β-turn of said toxin via at least two or more direct fusions or fusions made by a linker. Said exposed β-turn is meant herein as an accessible site that connects 2 β-strands of said β-strand-containing domain, wherein said exposed β-turn is different from the binding site of the target protein of said toxin, because any fusion of a scaffold to said binding site would render the fusion protein non-functional in its target binding. A toxin as used herein may also encompass toxin homologues, toxin variants, or toxin analogues, moreover, the toxin peptide may also be a peptidomimetic, or a synthetically produced or modified peptide. An embodiment provides a functional fusion protein wherein the toxin domain is fused with the scaffold protein in such a manner that the scaffold protein is “interrupting” the toxin domain its topology. In general, the “topology” of a protein refers to the orientation of regular secondary structures with respect to each other in three-dimensional space. Protein folds are defined mostly by the polypeptide chain topology (Orengo et al., 1994). So, at the most fundamental level, the ‘primary topology’ is defined as the sequence of secondary structure elements (SSEs), which is responsible for protein fold recognition motifs, and hence secondary and tertiary protein/domain folding. So in terms of protein structure, the true or primary topology is the sequence of SSEs, i.e. if one imagines of being able to hold the N- and C-terminal ends of a protein chain, and pull it out straight, the topology does not change whatever the protein fold. The protein fold is then described as the tertiary topology, in analogy with the primary and tertiary structure of a protein (also see Martin, 2000). The toxin domain of the fusion protein of the invention is hence interrupted in its primary topology, by introducing the scaffold protein fusion, but said toxin domain retained its tertiary structure allowing to retain its functional target binding capacity.

The “scaffold protein” refers to any type of protein which has a structure allowing a fusion with another protein, in particular with a toxin, as described herein. The classic principle of protein folding is that all the information required for a protein to adopt the correct three-dimensional conformation is provided by its amino acid sequence, resulting in specific folded proteins held together by various molecular interactions. To be useful as a scaffold herein, the scaffold protein must fold into distinct three-dimensional conformations. So, said scaffold protein is defined herein as a ‘folded’ protein, limiting the amino acid length to a minimum, because for short peptides it is generally known that these are very flexible, and not providing for a folded structure. So, the scaffold protein as used in the novel functional fusion proteins are inherently different from peptides or very small polypeptides, such as those composed of 40 amino acids or less, are not considered suitable scaffold proteins for fusing as a MegaToxin. So, the ‘scaffold protein’ as defined herein is a folded protein of at least 200 amino acids, or 150 amino acids, or at least 100 amino acids, or at least 50 amino acids, or more preferably at least 40 amino acids, at least 30 amino acids, at least 20 amino acids, at least 10 amino acids, at least 9 amino acids. Linkers or peptides, specifically linker of 8 or fewer amino acids are not suited as scaffold proteins for the purpose of the invention. Furthermore, such a “scaffold”, “junction” or “fusion partner” protein preferably has at least one exposed region in its tertiary structure to provide at least one accessible site to cleave as fusion point for the toxin. The scaffold polypeptide is used to assemble with the toxin domain and thereby results in the fusion protein in a docked configuration to increase mass, provide symmetry, and/or provide an enlarged toxin inducing a specific conformation state of the equivalent target and/or improve or add a functionality to the target. So, depending on the type of scaffold protein that is used, a different purpose of the resulting fusion protein is foreseen. The type and nature of the scaffold protein is irrelevant in that it can be any protein, and depending on its structure, size, function, or presence, the scaffold protein fused with said toxin domain as in the fusion protein of the invention will be of use in different application fields. The structure of the scaffold protein will impact the final chimeric structure, so a person skilled in the art should implement the known structural information on the scaffold protein and take into account its impact on the toxin properties of the fusion protein when selecting the scaffold. Examples of scaffold proteins are provided in the Examples of the present application as a basis to enable the skilled person to produce such MegaToxins, by selecting the scaffold and the fusion sites. A non-limiting number of scaffold proteins provided herein are enzymes, membrane proteins, receptors, adaptor proteins, chaperones, transcription factors, nuclear proteins, antigen-binding proteins themselves, such as Nanobodies, among others, may be applied as scaffold protein to create fusion proteins of the invention. In a specific embodiment, antigen-binding proteins such as antibodies or antibody-like proteins or derivatives thereof, such as Nanobodies or ISVDs are not suitable as a scaffold protein. In a preferred embodiment, the 3D-structure of said scaffold proteins is known or can be predicted or modelled by a skilled person, so the accessible sites to fuse the toxin domain with can be determined by said skilled person.

The novel chimeric or fusion proteins are fused in a unique manner to avoid that the junction is a flexible, loose, weak link/region within the chimeric protein structure. A convenient means for linking or fusing two polypeptides is by expressing them as a fusion protein from a recombinant nucleic acid molecule, which comprises a first polynucleotide encoding a first polypeptide operably linked to a second polynucleotide encoding the second polypeptide, in the classical known manner. In the recombinant nucleic acid molecule of the present invention however, the interruption of the topology of the toxin domain by said scaffold is also reflected in the design of the genetic fusion from which said fusion protein is expressed. So, in one embodiment, the functional fusion protein is encoded by a chimeric gene formed by recombining parts of a gene encoding for a protein toxin, and parts of a gene encoding the folded scaffold protein, wherein said encoded scaffold protein interrupts the primary topology of the encoded toxin domain at one or more accessible sites of an exposed β-turn of said toxin via at least two or more direct fusions or fusions made by encoded peptide linkers. So, the polynucleotides encoding the polypeptides to be fused are fragmented and recombined in such a way to provide the fusion protein that provides a rigid non-flexible link, connection or fusion between said proteins. The novel chimera are made by fusing the scaffold protein with the toxin domain in such a manner that the primary topology of the toxin domain is interrupted, meaning that the amino acid sequence of the toxin domain is interrupted at accessible site(s) of an exposed β-turn and joined to the accessible amino acid(s) of the scaffold protein, which sequence is therefore also possibly interrupted. The junctions are made intramolecularly, in other words internally within the amino acid sequences (see Examples and Figures). So, the recombinant fusions of the present invention result in functional chimera not solely fused at N- or C-termini, but comprising at least one internal fusion site, where the sites are fused directly or fused via a linker peptide. Where a circularly permutated scaffold is applied to produce the fusion protein, the amino acid sequence of said scaffold protein will be changed by connecting the N- and C-terminus, followed by a cleavage or separation of the amino acid sequence at another site within the sequence of the scaffold protein, corresponding to an accessible site in its tertiary structure, to be fused to the amino acid sequence of the toxin parts. Said N- and C-terminus connection for obtaining the circular permutation may be through a direct fusion, a linker peptide, or even via a short deletion of the region near N- and C-terminus followed by peptide bond of the ends.

The term “accessible site(s)”, “fusion site(s)” or “fusion point” or “connection site” or “exposed site”, are used interchangeably herein and all refer to amino acid sites of the protein sequence that are structurally accessible, preferably positions at the surface of the protein, or at exposed β-turns or loops in said β-strand-containing domain of said toxin, on the surface. A person skilled in the art will be able to determine those sites. The loops or (β)-turns involved in, or sterically hindering, the toxin target-binding sites should be avoided to be interrupted or cleaved for fusion to the scaffold as this may lead to loss of target-binding, hence loss of functionality, which is not suitable for the fusion proteins of the invention, and hence not intended to be applied here as accessible fusion site. So, with ‘accessible sites’ and ‘exposed regions’ as ‘loops’ or ‘beta turns’ as described herein is meant those sites and regions that are not the receptor sites or regions, which may differ in respect of the target. So, accessible sites can therefore include amino- and/or carboxy-terminal sites of the proteins, but the chimer cannot be exclusively based on fusion from accessible sites made up of N- or C-termini. At least one or more sites of the exposed β-turns or loops of the toxin domain are used for fusion to the scaffold protein as to result in an interruption of the topology of the known conventional domain fold. So, in one embodiment the at least one accessible site is not an N-terminal and/or C-terminal site of said domain if the at least one is one, and/or does not include an N- or C-terminal site of said domain. In a particular embodiment, the at least one site is not an N- or C-terminal amino acid of said domain. In another embodiment, the accessible site can be an N- or C-terminal site of the toxin, when at least more than one site is used to be fused to the scaffold protein. The scaffold protein is fused via accessible sites visible from its tertiary structure as well, for which in one embodiment, said at least one site is not an N- or C-terminal end of the scaffold protein, and in an alternative embodiment, the at least one site is the N- or C-terminal end of said scaffold.

More specifically, in one embodiment, the fusion protein is disclosed wherein the three-finger fold toxin is interrupted to insert the circularly permutated scaffold protein, in an exposed region at the accessible site of the beta turn that connects beta-strand β2 and β3 of said toxin domain.

In some embodiments of the invention, the fusions can be direct fusions, or fusions made by a linker peptide, said fusion sites being immaculately designed to result in a rigid, non-flexible fusion protein. In addition to the position of the selected accessible site(s), the length and type of the linker peptide contributes to the rigidity and possibly the functionality of the resulting fusion protein. Within the context of the present invention, the polypeptides constituting the fusion protein are fused to each other directly, by connection via a peptide bond, or indirectly, whereby indirect coupling assembles two polypeptides through connection via a short peptide linker. Preferred “linker molecules”, “linkers”, or “short polypeptide linkers” are peptides with a length of maximum ten amino acids, more likely four amino acids, typically is only three amino acids in length, but is preferably only two or even more preferred only a single amino acid to provide the desired rigidity to the junction of fusion at the accessible sites. Non-limiting examples of suitable linker sequences are described in the Example section, which can be randomized, and wherein linkers have been successfully selected to keep a fixed distance between the structural domains, as well as to maintain the fusion partners their independent functions (e.g. target-binding). In the embodiment relating to the use of rigid linkers, these are generally known to exhibit a unique conformation by adopting α-helical structures or by containing multiple proline residues. Under many circumstances, they separate the functional domains more efficiently than flexible linkers, which may as well be suitable, preferably in a short length of only 1-4 amino acids.

In one embodiment, the accessible site(s) of the toxin domain are in an exposed β-turn or loops of the domain fold. Said exposed β-turns or loops are identified as less fixed amino acid stretches, that are mostly located at the surface of the protein, and on the edges of a β-strand-containing domain structure. The most straightforward identification of “exposed regions” of the toxin domain are the exposed loops, preferably the β-turns, which are exposed loops located at the edges of the 13 sheet 3D-structure.

One embodiment relates to the functional fusion protein wherein the toxin comprises a β-strand-containing domain of at least three β-strands and wherein said scaffold protein interrupts the topology of the β-strand-containing domain at one or more accessible sites in an exposed β-turn of said at least 3 β-strand-containing domain. In a specific embodiment, said β-strand-containing domain of at least three β-strands comprises antiparallel β-strands. Said toxin may be a venom toxin. Furthermore, said toxin or venom toxin may comprise a three-finger fold domain. In a specific embodiment, said toxin comprising a three-finger fold domain is fused with the scaffold protein via inserting the scaffold protein in a β-turn that connects β-strand β2 and β-strand β3 of said three-finger fold domain of the toxin.

In another embodiment, the scaffold protein has a circular permutation. In a preferred embodiment, said circular permutation of the scaffold protein is present at the N- and/or C-terminus of the scaffold protein, or most preferably is between the N- and C-terminus of the scaffold protein. Another embodiment provides a scaffold protein comprising at least 2 anti-parallel β-strands.

A further aspect of the invention relates to a novel functional fusion protein comprising a toxin domain fused with a scaffold protein, wherein said scaffold protein interrupts the topology of said toxin domain, and wherein the total mass or molecular weight of the scaffold protein(s) is at least 30 kDa, so that the addition of mass and structural features by binding of the fusion to the target, such as the receptor of the ligand, will be significant and sufficient to allow 3-dimensional structural analysis of the target when non-covalently bound to said chimer. In another embodiment, the total mass or molecular weight of the scaffold protein(s) is at least 40, at least 45, at least 50, or at least 60 kDa. This particular size or mass increase will affect the signal-to-noise ratio in the images to decrease. Secondly, the chimer will offer a structural guide by providing adequate features for accurate image alignment for small or difficult to crystallize proteins to reach a sufficiently high resolution using cryo-EM and X-ray crystallography.

A further aspect of the invention relates to a nucleic acid molecule encoding said fusion protein of the present invention. Said nucleic acid molecule comprises the coding sequence of said toxin and said folded scaffold protein(s), and/or fragments thereof, wherein the interrupted topology of said domain is reflected in the fact that said domain sequence will contain an insertion of the scaffold protein sequence(s) (or a circularly permutated sequence, or a fragment thereof), so that the N-terminal toxin fragment and C-terminal toxin domain fragment are separated by the scaffold protein sequence or fragments thereof within said nucleic acid molecule. In another embodiment, a chimeric gene is described with at least a promoter, said nucleic acid molecule encoding the fusion protein, and a 3′ end region containing a transcription termination signal. Another embodiment relates to an expression cassette encoding said fusion protein of the present invention, or comprising the nucleic acid molecule or the chimeric gene encoding said fusion protein. Said expression cassettes are in certain embodiments applied in a generic format as a library, containing a large set of toxin fusions to select for the most suitable binders of the target. Further embodiments relate to vectors comprising said expression cassette or nucleic acid molecule encoding the fusion protein of the invention. In particular embodiments, vectors for expression in E. coli or other suitable expression hosts allow to produce the fusion proteins and purify them in the presence or absence of their targets. Alternative embodiments relate to host cells, comprising the fusion protein of the invention, or the nucleic acid molecule or expression cassette or vector encoding the fusion protein of the invention. In particular embodiments, said host cell further co-expresses the target protein or for instance receptor that specifically binds the toxin of the fusion protein. Another embodiment discloses the use of said host cells, or a membrane preparation isolated thereof, or proteins isolated therefrom, for ligand screening, drug screening, protein capturing and purification, or biophysical studies. The present invention providing said vectors further encompasses the option for high-throughput cloning in a generic fusion vector. Said generic vectors are described in additional embodiments wherein said vectors are specifically suitable for surface display in yeast, phages, bacteria or viruses. Furthermore, said vectors find applications in selection and screening of libraries comprising such generic vectors or expression cassettes with a large set of different ligands, in particular with different linkers for instance. So, the differential sequence in said libraries constructed for the screening of novel fusion protein for specific receptors is provided by the difference in the linker sequence, or alternatively in other regions.

In one embodiment, the vectors of the present invention are suitable to use in a method involving displaying a collection of toxin fusion proteins at the extracellular surface of a population of cells. Surface display methods are reviewed in Hoogenboom, (2005; Nature Biotechnol 23, 1105-16), and include bacterial display, yeast display, (bacterio)phage display. Preferably, the population of cells are yeast cells. The different yeast surface display methods all provide a means of tightly linking each fusion protein encoded by the library to the extracellular surface of the yeast cell which carries the plasmid encoding that protein. Most yeast display methods described to date use the yeast Saccharomyces cerevisiae, but other yeast species, for example, Pichia pastoris, could also be used. More specifically, in some embodiments, the yeast strain is from a genus selected from the group consisting of Saccharomyces, Pichia, Hansenula, Schizosaccharomyces, Kluyveromyces, Yarrowia, and Candida. In some embodiments, the yeast species is selected from the group consisting of S. cerevisiae, P. pastoris, H. polymorpha, S. pombe, K. lactis, Y. lipolytica, and C. albicans. Most yeast expression fusion proteins are based on GPI (Glycosyl-Phosphatidyl-Inositol) anchor proteins which play important roles in the surface expression of cell-surface proteins and are essential for the viability of the yeast. One such protein, alpha-agglutinin consists of a core subunit encoded by AGA1 and is linked through disulfide bridges to a small binding subunit encoded by AGA2. Proteins encoded by the nucleic acid library can be introduced on the N-terminal region of AGA1 or on the C-terminal or N-terminal region of AGA2. Both fusion patterns will result in the display of the polypeptide on the yeast cell surface.

The vectors disclosed herein may also be suited for prokaryotic host cells to surface display the proteins. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformnis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. When the host cell is a prokaryotic cell, examples of suitable cell surface proteins include suitable bacterial outer membrane proteins. Such outer membrane proteins include pili and flagella, lipoproteins, ice nucleation proteins, and autotransporters. Exemplary bacterial proteins used for heterologous protein display include LamB (Charbit et al., EMBO J, 5(11): 3029-37 (1986)), OmpA (Freudl, Gene, 82(2): 229-36 (1989)) and intimin (Wentzel et al., J Biol Chem, 274(30): 21037-43, (1999)). Additional exemplary outer membrane proteins include, but are not limited to, FliC, pullulunase, OprF, Oprl, PhoE, MisL, and cytolysin. An extensive list of bacterial membrane proteins that have been used for surface display are detailed in Lee et al., Trends Biotechnol, 21(1): 45-52 (2003), Jose, Appl Microbiol Biotechnol, 69(6): 607-14 (2006), and Daugherty, Curr Opin Struct Biol, 17(4): 474-80 (2007).

Furthermore, to allow an in-depth screening selection, vectors can be applied in yeast and/or phage display, followed FACS and panning, respectively. Display of toxin fusion proteins on yeast cells in combination with the resolving power of fluorescent-activated cell sorting (FACS), for instance, provides a preferred method of selection. In yeast display each toxin fusion protein is for instance displayed as a fusion to the Aga2p protein at 50.000 copies on the surface of a single cell. For selection by FACS, the labelling with different fluorescent dyes will determine the selection procedure. The fusion protein-displaying yeast library can next be stained with a mixture of the used fluorescent proteins. Two-colour FACS can then be used to analyse the properties of each fusion protein that is displayed on a specific yeast cell to resolve separate populations of cells. Yeast cells displaying a fusion protein that is highly suitable for binding the protein of interest, such as a receptor or antibody, will bind and can be sorted along the diagonal in a two-colour FACS. The use of vectors for such a selection method is most preferred when screening of fusion proteins specifically targeting a transient protein-protein interaction or conformation-selective binding state for instance. Similarly, vectors for phage display are applied, and used for display of the fusion proteins on the bacteriophages, followed by panning. Display can for instance be done on M13 particles by fusion of the toxin fusion proteins, within said generic vector, to phage coat protein III (Hoogenboom, 2000; Immunology today. 5699:371-378). For selection of fusion proteins specifically binding certain conformations and/or a transient protein-protein interaction for instance, only one of the interacting protomers is immobilized onto the solid phase. Bio-selection by panning of the phage-displayed fusion proteins is then performed in the presence of excess amounts of the remaining soluble protomer. Optionally, one can start with a round of panning on a cross-linked complex or protein that is immobilized on the solid phase.

Another aspect of the invention relates to a protein complex comprising said functional fusion protein, and a toxin target protein(s), wherein said target protein is specifically bound to the toxin fusion protein. More particular, wherein said target protein is bound to the toxin part of said fusion protein. More specifically a functional conformation may be bound and involve an agonist conformation, may involve a partial agonist conformation, or a biased agonist conformation, among others. Alternatively, a complex of the invention is disclosed, wherein the toxin of the fusion proteins stabilizes the target protein in a functional conformation, wherein said functional conformation is an inactive conformation, or wherein said functional conformation involves an inverse agonist conformation.

Another embodiment of the invention relates to a method of producing the toxin-containing functional fusion protein according to the invention comprising the steps of (a) culturing a host comprising the vector, expression cassette, chimeric gene or nucleic acid sequence of the present invention, under conditions conducive to the expression of the fusion protein, and (b) optionally, recovering the expressed polypeptide.

Another aspect relates to the use of the toxin fusion protein of the present invention or of the use of the nucleic acid molecule, chimeric gene, the expression cassette, the vectors, or the complex, in structural analysis of its target protein. In particular, the use of the fusion protein in structural analysis of a target protein wherein said target protein is a protein specifically bound to said toxin part of said fusion protein. “Solving the structure” or “structural analysis” as used herein refers to determining the arrangement of atoms or the atomic coordinates of a protein, and is often done by a biophysical method, such as X-ray crystallography or cryogenic electron-microscopy (cryo-EM). Specifically, an embodiment relates to the use in structural analysis comprising single particle cryo-EM or comprising crystallography. The use of such toxin-containing fusion proteins of the present invention in structural biology renders the major advantage to serve as crystallization aids, namely to play a role as crystal contacts and to increase symmetry, and even more to be applied as rigid tools in Cryo-EM, which will be very valuable to solve large structures of difficult targets or complex visualization, to reduce size barriers coped with today, also to increase symmetry, and to stabilize and visualize specific conformational states of the target in complex with said toxin fusion protein.

Using cryo-EM for structure determination has several advantages over more traditional approaches such as X-ray crystallography. In particular, cryo-EM places less stringent requirements on the sample to be analysed with regard to purity, homogeneity and quantity. Importantly, cryo-EM can be applied to targets that do not form suitable crystals for structure determination. A suspension of purified or unpurified protein, either alone or in complex with other proteinaceous molecules can be applied to carbon grids for imaging by cryo-EM. The coated grids are flash-frozen, usually in liquid ethane, to preserve the particles in the suspension in a frozen-hydrated state. Larger particles can be vitrified by cryofixation. The vitrified sample can be cut in thin sections (typically 40 to 200 nm thick) in a cryo-ultramicrotome, and the sections can be placed on electron microscope grids for imaging. The quality of the data obtained from images can be improved by using parallel illumination and better microscope alignment to obtain resolutions as high as ˜3.3 Å. At such a high resolution, ab initio model building of full-atom structures is possible. However, lower resolution imaging might be sufficient where structural data at atomic resolution on the chosen or a closely related target protein and the selected heterologous protein or a close homologue are available for constrained comparative modelling. To further improve the data quality, the microscope can be carefully aligned to reveal visible contrast transfer function (CTF) rings beyond ⅓ Å⁻¹ in the Fourier transform of carbon film images recorded under the same conditions used for imaging. The defocus values for each micrograph can then be determined using software such as CTFFIND.

A method for determining a 3-dimensional structure of a functional fusion protein as described herein in complex with a toxin target protein comprising the steps of: (i) providing the fusion protein according to the invention, and providing the toxin target to form a complex, wherein said target protein is bound to the toxin part of the fusion protein of the invention, or providing the functional complex as described herein above; (ii) display said complex in suitable conditions for structural analysis, wherein the 3D structure of said protein complex is determined at high-resolution.

In a specific embodiment, said structural analysis is done via X-ray crystallography. In another embodiment, said 3D analysis comprises Cryo-EM. More specifically, a methodology for Cryo-EM analysis is described here as follows. A sample (e.g. the fusion protein of choice in a complex with a target of interest), is applied to a best-performing discharged grid of choice (carbon-coated copper grids, C-Flat, 1.2/1.3 200-mesh: Electron Microscopy Sciences; gold R1.2/1.3 300 mesh UltraAuFoil grids: Quantifoil; etc.) before blotting, and then plunge-frozen in to liquid ethane (Vitrobot Mark IV (FEI) or other plunger of choice). Data for a single grid are collected at 300 kV Electron Microscope (Krios 300 kV as an example with supplemented phase plate of choice) equipped with a detector of choice (Falcon 3EC direct-detector as an example). Micrographs are collected in electron-counting mode at a proper magnification suitable for an expected ligand/receptor complex size. Collected micrographs are manually checked before further image processing. Apply drift correction, beam induced motion, dose-weighting, CTF fitting and phase shift estimation by a software of choice (RELION, SPHIRE packages as examples). Pick particles with a software of choice and use them for to 2D classification. Manually-inspected 2D classes and remove false positives. Bin particles accordingly to data collection settings. Generate an initial 3D reference model by applying a proper low-pass filter and generate a number (six as an example) of 3D classes. Use original particles for 3D refinement (if needed use soft mask). Estimate a reconstruction resolution by using Fourier Shell Correlation (FSC)=0.143 criterion. Local resolution can be calculated by the MonoRes implementation in Scipion. Reconstructed cryo-EM maps can be analyzed using UCSF Chimera and Coot software. The design model can be initially fitted using UCSF Chimera and analyzed by software of choice (UCSF Chimera, PyMOL or Coot).

Another advantage of the method of the invention is that structural analysis, which is in a conventional manner only possible with highly pure protein, is less stringent on purity requirements thanks to the use of the toxin fusion proteins. Such toxin-containing functional fusion proteins will specifically filter out the target of interest via its high affinity binding site, within a complex mixture. The target protein can in this way be trapped, frozen and analysed via cryo-EM.

Said method is in alternative embodiments also suitable for 3D analysis wherein the receptor protein is a transient protein-protein complex or is in a transient specific conformational state. Additionally, said fusion protein molecules can also be applied in a method for determining the 3-dimensional structure of a target to stabilize transient protein-protein interactions as targets to allow their structural analysis.

Another embodiment relates to a method to select or to screen for a panel of functional fusion proteins binding to different conformations of the same toxin target protein, comprising the steps of: (i) designing a library of fusion proteins binding the target protein, and (ii) selecting the fusion proteins via surface yeast display, phage display or bacteriophages to obtain a fusion protein panel comprising proteins binding to several relevant conformational states of said receptor protein, thereby allowing several conformations of the target protein to be analysed in for instance cryo-EM in separate images. To obtain specific or certain conformational states, one can make use of cell-based systems wherein the receptor is on the membrane, wherein said cells may be treated or manipulated according to the purpose of the experiment.

In another embodiment, said method and said functional fusion protein of the invention is used for structure-based drug design and structure-based drug screening. The iterative process of structure-based drug design often proceeds through multiple cycles before an optimized lead goes into phase I clinical trials. The first cycle includes the cloning, purification and structure determination of the receptor protein or nucleic acid by one of three principal methods: X-ray crystallography, NMR, or homology modelling. Using computer algorithms, compounds or fragments of compounds from a database are positioned into a selected region of the structure. One could use the fusion protein of the invention to fix or stabilize certain structural conformations of a target. The selected compounds are scored and ranked based on their steric and electrostatic interactions with this target site, and the best compounds are tested with biochemical assays. In the second cycle, structure determination of the target in complex with a promising lead from the first cycle, one with at least micromolar inhibition in vitro, reveals sites on the compound that can be optimized to increase potency. Also at this point, the functional fusion protein of the invention may come into play, as it facilitates the structural analysis of said toxin target protein in a certain conformational state. Additional cycles include synthesis of the optimized lead, structure determination of the new target:lead complex, and further optimization of the lead compound. After several cycles of the drug design process, the optimized compounds usually show marked improvement in binding and, often, specificity for the target. A library screening leads to hits, to be further developed into leads, for which structural information as well as medicinal chemistry for Structure-Activity-Relationship analysis is essential.

In a final aspect of the present invention, the functional fusion protein as described herein is used as a medicament or therapeutic, preferably in a pharmaceutical composition. The term “medicament”, as used herein, refers to a substance/composition used in therapy, i.e., in the prevention or treatment of a disease or disorder. According to the invention, the terms “disease” or “disorder” refer to any pathological state, in particular to the diseases or disorders as defined herein. Although several applications for clinical purpose using natural toxins face issues of immunogenicity, certain applications may benefit from these novel functional fusions proteins as provided herein to further develop for therapeutic purposes. For instance, ion channel targeting in the field of neurodegenerative disorders may be treated using the functional fusion proteins of the present invention, wherein venomous animal toxins modulate for instance ion channel function. Depending on the type of scaffold protein of the toxin-containing functional fusion proteins, the suitability for clinical or medical use will be acceptable for treating pathological progress of neurodegenerative disorders and provide good candidates for new drug development. Neurodegeneration is the progressive disease resulting in the loss of structures or functions, and the final lethal destiny of neurons. Neurodegenerative diseases including Parkinson's disease (PD), Alzheimer's disease (AD), Huntington's disease, epilepsy, multiple sclerosis, amyotrophic lateral sclerosis, etc., affect millions of individuals worldwide. An embodiment of the invention provides for a composition, or a pharmaceutical composition, comprising the functional fusion protein as described herein.

When a fusion protein as described herein is used as a medicament, the scaffold protein may be conjugated to a half-life extension module, or may function as a half-life extension module itself. Such modules are known to a person skilled in the art and include, for example, albumin, an albumin-binding domain, an Fc region/domain of an immunoglobulins, an immunoglobulin-binding domain, an FcRn-binding motif, and a polymer. Particularly preferred polymers include polyethylene glycol (PEG), hydroxyethyl starch (HES), hyaluronic acid, polysialic acid and PEG-mimetic peptide sequences. Modifications preventing aggregation of the isolated (poly-)peptides are also known to the skilled person and include, for example, the substitution of one or more hydrophobic amino acids, preferably surface-exposed hydrophobic amino acids, with one or more hydrophilic amino acids. In one embodiment, the isolated (poly-)peptide or the immunogenic variant thereof or the immunogenic fragment of any of the foregoing, comprises the substitution of up to 10, 9, 8, 7, 6, 5, 4, 3 or 2, preferably 5, 4, 3 or 2, hydrophobic amino acids, preferably surface-exposed hydrophobic amino acids, with hydrophilic amino acids. Preferably, other properties of the isolated (poly-)peptide, e.g., its immunogenicity, antigen-binding functionality, are not compromised by such substitution.

A “patient” or “subject”, for the purpose of this invention, relates to any organism such as a vertebrate, particularly any mammal, including both a human and another mammal, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, also designated “patient” herein.

The term “preventing”, as used herein, may refer to stopping/inhibiting the onset of a disease or disorder (e.g., by prophylactic treatment). It may also refer to a delay of the onset, reduced frequency of symptoms, or reduced severity of symptoms associated with the disease or disorder (e.g., by prophylactic treatment). The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders.

The pharmaceutical composition as described herein can be utilized to achieve the desired pharmacological effect by administration to a patient in need thereof. The present invention includes pharmaceutical compositions that are comprised of a pharmaceutically acceptable carrier and a pharmaceutically effective amount of a compound, or salt thereof, of the present invention. A pharmaceutically effective amount of compound is preferably that amount which produces a result or exerts an influence on the particular condition being treated. In general, “therapeutically effective amount”, “therapeutically effective dose” and “effective amount” means the amount needed to achieve the desired result or results. One of ordinary skill in the art will recognize that the potency and, therefore, an “effective amount” can vary depending on the identity and structure of the compound of the invention. One skilled in the art can readily assess the potency of the compound. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. Such ingredients and procedures include those described in the following references, each of which is incorporated herein by reference: Powell, M. F. et al. (“Compendium of Excipients for Parenteral Formulations” PDA Journal of Pharmaceutical Science & Technology 1998, 52(5), 238-311), Strickley, R. G (“Parenteral Formulations of Small Molecule Therapeutics Marketed in the United States (1999)-Part-1” PDA Journal of Pharmaceutical Science & Technology 1999, 53(6), 324-349), and Nema, S. et al. (“Excipients and Their Use in Injectable Products” PDA Journal of Pharmaceutical Science & Technology 1997, 51 (4), 166-171).

The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

The functional fusion protein of the invention can be administered with pharmaceutically acceptable carriers well known in the art using any effective conventional dosage form, including immediate, slow and timed release preparations, and can be administered by any suitable route such as any of those commonly known to those of ordinary skill in the art. For therapy, the pharmaceutical composition of the invention can be administered to any patient in accordance with standard techniques.

It is to be understood that although particular embodiments, specific configurations as well as materials and/or molecules, have been discussed herein for engineered cells and methods according to the disclosure, various changes or modifications in form and detail may be made without departing from the scope of this invention. The following examples are provided to better illustrate particular embodiments, and they should not be considered limiting the application. The application is limited only by the claims.

### EXAMPLES

General

We have designed rigid fusion proteins, also called ‘MegaToxins’ (Mts), consisting of a toxin and a scaffold protein, wherein the toxin globular core domain, comprising at least three β-strands, is connected to the scaffold protein via two or three short linkers, or via two or three direct linkages, at an exposed β-turn. Depending on the mechanism of action and interaction or binding mode of the toxin with its target, these rigid fusion proteins bind and fix specific and different conformational states of the toxin target. Those MegaToxin fusion proteins represent enlarged toxin ligands and are instrumental as next-generation chaperones for determining protein structures of toxin complexes (with their targets or interactors such as receptors or ion channels for instance), by aiding in several applications including X-ray crystallography and cryo-EM. The MegaToxins function as next generation chaperones by reducing the conformational flexibility of the bound partner and by extending the surfaces predisposed to forming crystal contacts, as well as by providing additional phasing information. By mixing a specific MegaToxin fusion protein with its target, their specific binding interaction leads to “mass” addition and fixing a specific conformational state of the receptor. To design functional MegaToxin fusion protein variants, in silico molecular modelling using Modeler software (https://salilab.org/modeller) was used. Several low free energy MegaToxins were generated. As a proof of concept of this approach, we used three different scaffold proteins, a circularly permutated variant (c7HopQ) of the gene encoding the adhesion domain of HopQ (a periplasmic protein from H. pylori, PDB 5LP2, SEQ ID NO:16) and a circularly permutated variant c1 and variant c2 of the 86 kDa periplasmic protein of E. coli YgjK (PDB 3W7S, SEQ ID NO: 5). These scaffold proteins have been inserted in the β-turn between β-strand 2 (β2) and the β-strand 3 (β3) of the three-finger-fold toxins alpha-cobratoxin (binding the Acetylcholine receptor) (Example 1 and 3), alpha-bungarotoxin (Example 2, 5, 6, and 7), and micrurotoxin1 (Example 4, 8, and 9). Moreover, the RCT plant-originating toxin has been used in Example 11 to provide for a fusion using the HopQ scaffold, as well as the sea-anemone Stichlysin venom toxin (Example 10), and a neurotoxin from scorpion has been fused according to the invention to obtain a fusion with Ts1 in Example 12. The toxin-based fusion proteins were demonstrated to be expressed as secreted proteins in the periplasm of E. coli (Example 2, 8 and 9), and/or in or on the surface of yeast cells (Example 5 and 7), which allowed FACS sorting and determination of the binding capacity to specific antibodies or targets (Example 6 and 7)

### Example 1: Design and Generation of a 50 kDa Fusion Protein Built from a c7HopQ Scaffold Inserted into the β-Strand β2-β3-Connecting β-Turn of Alpha-Cobratoxin

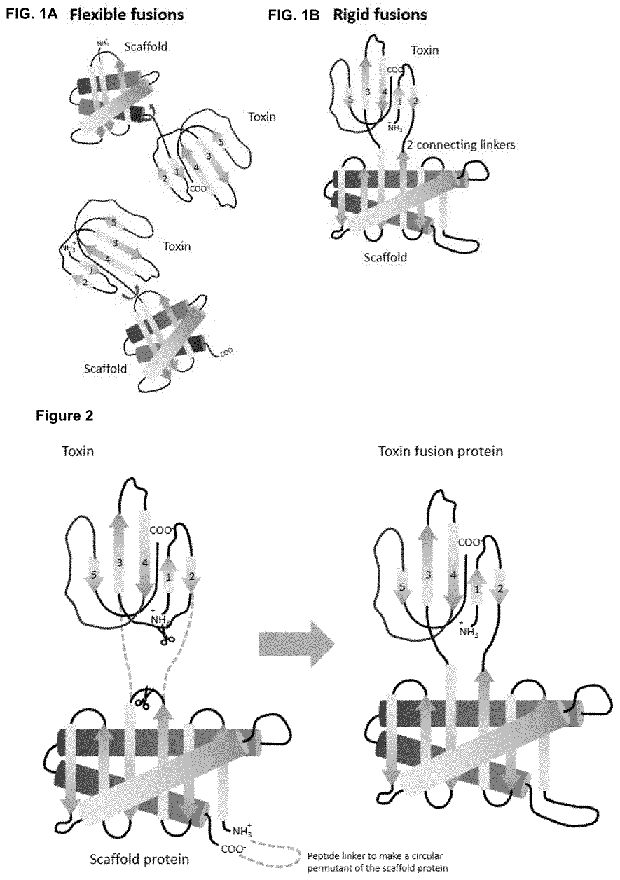

As a first proof of concept of obtaining rigid fusion proteins ‘MegaToxins’, alpha-cobratoxin was grafted onto a large scaffold protein via two peptide bonds that connect alpha-cobratoxin to a scaffold according to FIG. 2 to build a rigid MegaToxin. The 50 kDa MegaToxin described here is a chimeric polypeptide concatenated from parts of the toxin and parts of a scaffold protein connected according to FIGS. 2 and 3. Here, the toxin used is the alpha-cobratoxin (binding the Acetylcholine receptor) as depicted in SEQ ID NO:1 (PDB: 1YI5). The scaffold protein was inserted in the β-turn connecting β-strand 2 and β-strand 3 of the alpha-cobratoxin. The scaffold protein is an adhesin domain of Helicobacter pylori strain G27 (PDB: 5LP2; SEQ ID NO:16) called HopQ (Javaheri et al, 2016). The N- and C-terminus of HopQ was connected, although after a truncation of 7 amino acids in the circular permutation region (called c7HopQ) which otherwise appeared as a loop never fully visible in electron density of crystal structures. This truncated fusion creates a circularly permutated variant of HopQ, called c7HopQ, wherein a cleavage within the amino acid sequence was made somewhere else in its sequence (i.e. in a position corresponding to an accessible site in an exposed region of said scaffold protein). A low free energy Mt_{alpha-cobratoxin}^{c7HopQ} (SEQ ID NO:2) was generated, where all parts were connected as follows: the N-terminus until β-strand 2 of the alpha-cobratoxin (1-14 of SEQ ID NO:1), a C-terminal part of HopQ (residues 192-411 of SEQ ID NO: 16), an N-terminal part of HopQ (residues 18-185 of SEQ ID NO:16), the C-terminal part from β-strand 3 till end of the alpha-cobratoxin (17-68 of SEQ ID NO:1), 6×His tag and EPEA tag (U.S. Pat. No. 9,518,084 B2).

We set out to express the 50 kDa fusion protein in the periplasm of E. coli, purified it to homogeneity and determined its properties. In order to express MegaToxin Mt_{alpha-cobratoxin}^{c7HopQ} in the periplasm of E. coli, we used standard methods to construct a vector that allowed the expression of alpha-cobra MegaToxins: scaffolds can be inserted into the β-turn connecting β-strand 2 (β2) and β-strand 3 (β3) of alpha-cobratoxin. The vector is a derivative of pMESy4 (Pardon et al., 2014) and contains an open reading frame that encodes the following polypeptides: the DsbA leader sequence that directs the secretion of the MegaToxin to the periplasm of E. coli, the N-terminus until β-strand β2 of the alpha-cobratoxin, the circularly permutated variant of HopQ (c7HopQ), the C-terminus from β-strand β3 of the alpha-cobratoxin, the 6×His tag and the EPEA tag followed by the Amber stop codon.

### Example 2: Design and Generation of a 50 kDa Fusion Protein Built from a c7HopQ Scaffold Inserted into the β-Strand β2-β3-Connecting β-Turn of Alpha-Bungarotoxin

As a second proof of concept of obtaining rigid fusion proteins ‘MegaToxins’, alpha-bungarotoxin was grafted onto a large scaffold protein via two peptide bonds that connect alpha-bungarotoxin (BgTX) to a scaffold according to FIG. 2 to build a rigid MegaToxin. The 50 kDa MegaToxin described here is a chimeric polypeptide concatenated from parts of the toxin and parts of a scaffold protein connected according to FIGS. 2 and 4. Here, the toxin used is the alpha-bungarotoxin (binding cholinergic receptors) as depicted in SEQ ID NO:3 (PDB 4UY2). The scaffold protein was inserted in the β-turn connecting β-strand 2 and β-strand 3 of the alpha-bungarotoxin. The scaffold protein is an adhesin domain of Helicobacter pylori strain G27 (PDB: 5LP2; SEQ ID NO:16) called HopQ. The N- and C-terminus of HopQ was connected, although after a truncation of 7 amino acids in the circular permutation region (called c7HopQ) which otherwise appeared as a loop never fully visible in electron density of crystal structures. This truncated fusion creates a circularly permutated variant of HopQ, called c7HopQ, wherein a cleavage within the amino acid sequence was made somewhere else in its sequence (i.e. in a position corresponding to an accessible site in an exposed region of said scaffold protein). A low free energy Mt_{BgTx}^{c7HopQ} (SEQ ID NO:4) was generated, where all parts were connected as follows: the N-terminus until β-strand 2 of the alpha-bungarotoxin (1-17 of SEQ ID NO:3), a C-terminal part of HopQ (residues 193-411 of SEQ ID NO:16), an N-terminal part of HopQ (residues 18-185 of SEQ ID NO:16), the C-terminal part from β-strand 3 till end of the alpha-bungarotoxin (20-73 of SEQ ID NO:3), 6×His tag and EPEA tag (U.S. Pat. No. 9,518,084 B2).

We demonstrated that the MegaToxins Mt_{BgTx}^{c7HopQ} (SEQ ID NO:4) can be expressed as a well-folded protein on the surface of yeast, followed by clone selection via fluorescence-activated cell sorting (FACS; see Example 5).

We set out to express the 50 kDa fusion protein in the periplasm of E. coli, purified it to homogeneity and determined its properties. In order to express MegaToxin Mt_{alpha-bungarotoxin}^{c7HopQ} in the periplasm of E. coli, we used standard methods to construct a vector that allowed the expression of alpha-bungarotoxin MegaToxins: scaffolds can be inserted into the β-turn connecting β-strand 2 (β2) and β-strand 3 (β3) of alpha-bungarotoxin. The vector is a derivative of pMESy4 (Pardon et al., 2014) and contains an open reading frame that encodes the following polypeptides: the DsbA leader sequence that directs the secretion of the MegaToxin to the periplasm of E. coli, the N-terminus until β-strand β2 of the alpha-bungarotoxin, the circularly permutated variant of HopQ (c7HopQ), the C-terminus from β-strand β3 of the alpha-bungarotoxin, the 6×His tag and the EPEA tag followed by the Amber stop codon. The expression and purification of the Mt_{BgTx}^{c7HopQ} was done as described by Pardon et al. (2014).

Two of the selected Mt_{BgTx}^{c7HopQ} clones (called MP1583_8 and MP1583_E7) were expressed in the periplasm of E. coli, purified and analysed on SDS_PAGE and Western blot (FIG. 16).

IMAC and SEC purified samples were separated on 12% SDS-PAGE gels in duplicate. After electrophoresis, proteins from one gel were colored with Coomassie blue (FIGS. 16A and C) while the proteins of the other gel were transferred to a nitrocellulose membrane. This membrane was blocked with 4% skimmed milk. Expression of recombinant Mt_{BgTx}^{c7HopQ} was detected using the biotinylated anti-EPEA (Life Technologies Cat. NO. 7103252100) as the primary antibody and a streptavidin-alkaline phosphatase conjugate (Promega, Cat. NO. V5591) in combination with NBT and BCIP to develop the blot (FIGS. 16B and D). The detection of bands with the appropriate molecular weight (approximately 50 kDa for the Mt_{BgTx}^{c7HopQ}) confirms expression of the MegaToxin fusion protein for all constructs generated.

### Example 3: Design and Generation of a 94 kDa Fusion Protein Built from a c2YgjK Scaffold Inserted into the β-Strand β2-β3-Connecting β-Turn of Alpha-Cobratoxin

As a next example of obtaining rigid fusion proteins ‘MegaToxins’, alpha-cobratoxin was grafted onto a large scaffold protein via two peptide bonds that connect alpha-cobratoxin to a scaffold according to FIG. 2 to build a rigid MegaToxin. The 94 kDa MegaToxin described here is a chimeric polypeptide concatenated from parts of the toxin and parts of a scaffold protein connected according to FIGS. 2 and 5. Here, the toxin used is the alpha-cobratoxin (binding the Acetylcholine receptor) as depicted in SEQ ID NO:1 (PDB: 1YI5). The scaffold protein was inserted in the β-turn connecting β-strand 2 and β-strand 3 of the alpha-cobratoxin. The alternative scaffold protein used was YgjK, a 86 kDa periplasmic protein of E. coli (PDB 3W7S, SEQ ID NO: 5). To create Mt_{alpha-cobratoxin}^{c2YgjK} variants all parts were connected to each other from the amino to the carboxy terminus in the next given order by peptide bonds (SEQ ID NO:6-9): the N-terminus until β-strand 2 of the alpha-cobratoxin (1-14 of SEQ ID NO:1), a peptide linker of one or two amino acids with random composition, the C-terminal part of YgjK (residues 106-760 of SEQ ID NO: 5), a short peptide linker (SEQ ID NO: 10) connecting the C-terminus and the N-terminus of YgjK to produce a circular permutant of the scaffold protein, the N-terminal part of YgjK (residues 1-100 of SEQ ID NO:5), a peptide linker of one or two amino acids with random composition, the C-terminal part from β-strand 3 till end of the alpha-cobratoxin (17-68 of SEQ ID NO:1), 6×His tag and EPEA tag (U.S. Pat. No. 9,518,084 B2).

We set out to express the 94 kDa fusion protein in the periplasm of E. coli, purified it to homogeneity and determined its properties. In order to express MegaToxin Mt_{alpha-cobratoxin}^{c2YgjK} in the periplasm of E. coli, we used standard methods to construct a vector that allowed the expression of alpha-cobra MegaToxins: scaffolds can be inserted into the β-turn connecting β-strand 2 (β2) and β-strand 3 (β3) of alpha-cobratoxin. The vector is a derivative of pMESy4 (Pardon et al., 2014) and contains an open reading frame that encodes the following polypeptides: the pelB leader sequence that directs the secretion of the MegaToxin to the periplasm of E. coli, the N-terminus until β-strand β2 of the alpha-cobratoxin, the circularly permutated variant of YgjK (c2YgjK), the C-terminus from β-strand β3 of the alpha-cobratoxin, the 6×His tag and the EPEA tag followed by the Amber stop codon.

### Example 4: Design and Generation of a 94 kDa Fusion Protein Built from a c2YgjK Scaffold Inserted into the β-Strand β2-β3-Connecting β-Turn of Micrurotoxin1 (MmTX1)

As a next example of obtaining rigid fusion proteins ‘MegaToxins’, micrurotoxin1 was grafted onto a large scaffold protein via two peptide bonds that connect micrurotoxin1 to a scaffold according to FIG. 2 to build a rigid MegaToxin. The 94 kDa MegaToxin described here is a chimeric polypeptide concatenated from parts of the toxin and parts of a scaffold protein connected according to FIGS. 2 and 6. Here, the toxin used is the micrurotoxin1 (binding the GABA_{A} receptor(s)) as depicted in SEQ ID NO:11 (a structural homologue of bungarotoxin PDB 4UY2). The scaffold protein was inserted in the (3-turn connecting β-strand 2 and β-strand 3 of the micrurotoxin1. The scaffold protein used was YgjK, a 86 kDa periplasmic protein of E. coli (PDB 3W7S, SEQ ID NO: 5). To create Mt_{micrurotoxin1}^{c2YgjK} variants all parts were connected to each other from the amino to the carboxy terminus in the next given order by peptide bonds (SEQ ID NO:12-15): the N-terminus until β-strand 2 of the micrurotoxin1 (1-18 of SEQ ID NO:11), a peptide linker of one or two amino acids with random composition, the C-terminal part of YgjK (residues 106-760 of SEQ ID NO: 5), a short peptide linker (SEQ ID NO: 10) connecting the C-terminus and the N-terminus of YgjK to produce a circular permutant of the scaffold protein, the N-terminal part of YgjK (residues 1-100 of SEQ ID NO:5), a peptide linker of one or two amino acids with random composition, the C-terminal part from β-strand 3 till end of the micrurotoxin1 (21-64 of SEQ ID NO:11), 6×His tag and EPEA tag (U.S. Pat. No. 9,518,084 B2).

We set out to express the 94 kDa fusion protein in the periplasm of E. coli, purified it to homogeneity and determined its properties. In order to express MegaToxin Mt_{micrurotoxin1}^{c2YgjK} in the periplasm of E. coli, we used standard methods to construct a vector that allowed the expression of micrurotoxin1 MegaToxins: scaffolds can be inserted into the β-turn connecting β-strand 2 (β2) and β-strand 3 (β3) of micrurotoxin1. The vector is a derivative of pMESy4 (Pardon et al., 2014) and contains an open reading frame that encodes the following polypeptides: the pelB leader sequence that directs the secretion of the MegaToxin to the periplasm of E. coli, the N-terminus until β-strand β2 of micrurotoxin1, the circularly permutated variant of YgjK (c2YgjK), the C-terminus from β-strand β3 of the micrurotoxin1, the 6×His tag and the EPEA tag followed by the Amber stop codon.

### Example 5: Fluorescence-Activated Cell Sorting to Select EBY100 Yeast Cells Displaying MegaToxin Mt_{BgTx}^{c7HopQ} on the Cell Surface

To demonstrate that MegaToxin Mt_{BgTx}^{c7HopQ} (SEQ ID NO:4) can be expressed as a correctly folded protein, we displayed this MegaToxin on the surface of yeast (Boder, 1997) and examined the specific binding of anti-bungarotoxin polyclonal antibodies to yeast cells displaying this MegaToxin by flow cytometry. In order to display the Mt_{BgTx}^{c7HopQ} (SEQ ID NO:4) on yeast, we used standard methods to construct an open reading frame that encodes the MegaToxin in fusion to a number of accessory peptides and proteins (SEQ ID NO:22): the appS4 leader sequence that directs extracellular secretion in yeast (Rakestraw, 2009), MegaToxin Mt_{BgTx}^{c7HopQ}, a flexible peptide linker, the Aga2p the adhesion subunit of the yeast agglutinin protein Aga2p which attaches to the yeast cell wall through disulfide bonds to Aga1p protein, an acyl carrier protein for the orthogonal fluorescent staining of the displayed fusion protein (Johnsson, 2005) followed by the cMyc Tag. This open reading frame was put under the transcriptional control of galactose-inducible GAL1/10 promotor into a variant of the pNACP vector (Uchański, 2019) and introduced into yeast strain EBY100.

EBY100 yeast cells, bearing this plasmid, were grown and induced overnight in a galactose-rich medium to trigger the expression and secretion of the MegaToxin-Aga2p-ACP fusion. The expression of MegaToxin Mt_{BgTx}^{c7HopQ} on the surface of yeast is induced by changing growing conditions from glucose-rich to galactose-rich media. For in vitro selection by yeast display and fluorescence-activated cell sorting, induced yeast cells were stained, washed and subjected to flow-cytometry, the presence of the MegaToxin, displayed on the cell, was examined by the specific binding of anti-bungarotoxin polyclonal antibodies. The induced EBY100 yeast cells were incubated with anti-bungarotoxin polyclonal antibodies. After washing these cells, the cells were stained with anti-rabbit-FITC. At the same time the cells were incubated with an anti-HopQ nanobody labelled with Alexa fluor 647 to detect the presence of the HopQ scaffold. Indeed, in the two-dimensional flow cytometry, we observed a clear shift in both the FITC-fluorescence level as the 647-fluorescence level, indicating the presence of bungarotoxin as well as the c7HopQ (FIG. 14A). Cells falling in the β2 gate of FIG. 14A, were sorted, grown at 30° C. on SDCAA plates and sequence analysed to determine the amino acids in both linkers, linking the toxin to the scaffold (FIG. 14B). Four individual clones with different linkers were grown, induced, fluorescently stained and examined by flow cytometry (FIGS. 15A-15C). When yeast cells were stained as described above (FIG. 15A), the two-dimensional flow cytometric analysis confirmed the shift in the FITC-fluorescence (detection of BgTX) level as well as the shift in the 647-fluorescence (presence op cHopQ) level. In contrast, when the clones were stained with anti-HA in the same way only a shift in the 647-fluorescence (presence op cHopQ) level was seen (FIG. 15B). We conclude from these experiments that MegaToxin Mt_{BgTx}^{c7HopQ} can be expressed as a chimeric protein on the surface of yeast.

### Example 6: Binding of GABA_{A}R to MegaToxin Mt_{BgTx}^{c7HopQ}

The Mt_{BgTx}^{c7HopQ} fusion proteins, expressed in E. coli and purified (see Example 5), were spotted (0.5 and 2 μg) in quadruplicate on a nitrocellulose membranes next to 0.5 and 2 μg of het pentameric β3 GABA_{A}R. This membrane was blocked with 4% skimmed milk. The Mt_{BgTx}^{c7HopQ} fusion proteins carry a His and EPEA tag and can be detected by an anti-EPEA antibody, while the GABA_{A}R carries a 1D4-tag which can be detected with the anti-1D4 monoclonal antibody. The dot blot set-up can be seen in FIG. 17A. Strip 1 is incubated with the Mt_{BgTx}^{c7HopQ}, strip 2 is not incubated with the Mt_{BgTx}^{c7HopQ} and serves as a negative control for the binding to GABA_{A}R. The EPEA-tag of the MegaToxin was detected using the biotinylated anti-EPEA (Life Technologies Cat. NO. 7103252100) as the primary antibody and a streptavidin-alkaline phosphatase conjugate (Promega, V5591) in combination with NBT and BCIP to develop the blot. If the MegaToxin is able to bind to the GABA_{A}R, signals should be seen on spotted GABA_{A}R and on the spotted Mt_{BgTx}^{c7HopQ} serving as a positive control. Strip 3 is incubated with the GABA_{A}R, strip 4 is not incubated with the GABA_{A}R, and serves as a negative control for the binding to the Mt_{BgTx}^{c7HopQ}. The 1D4-tag of the GABA_{A}R was detected using the anti 1D4 monoclonal Ab (Sigma Cat. NO 5403) as the primary antibody and an anti-mouse-alkaline phosphatase conjugate (Sigma Cat. NO A3562) in combination with NBT and BCIP to develop the blot. If the GABA_{A}R is able to bind the MegaToxin, signals should be seen on the spotted Mt_{BgTx}^{c7HopQ} and on the spotted GABA_{A}R that serves as positive control in strips 3 and 4.

In FIG. 17B, Mt_{BgTx}^{c7HopQ}_A8 was spotted onto nitrocellose, next to the GABA_{A}R β3, and in FIG. 17C Mt_{BgTx}^{c7HopQ}_E7 was spotted onto nitrocelluse, next to the GABA_{A}R β3. When the GABA_{A}R β3 pentameric protein was spotted and incubated with the MegaToxins, no binding could be seen, only the directly spotted MegaToxins could be detected with anti-EPEA. In contrast when the MegaToxins were spotted on the membranes and these we incubated with GABA_{A}R β3 pentameric protein, binding of the GABA_{A}R β3 to the MegaToxin could be detected by using the anti-1D4-tag for both MegaToxins (next to the directly spotted GABA_{A}R that served as a positive control). We can conclude that the Mt_{BgTx}^{c7HopQ} are well-folded and functional in that these MegaToxins are able to bind to the GABA_{A}R β3 homopentamer target.

### Example 7: Design and Generation of a 95 kDa Fusion Protein Built from a c2YgjK Scaffold Inserted into β-Turn Connecting the β-Strands β2 and β3 of Alpha-Bungarotoxin

As a next example of obtaining rigid fusion proteins ‘MegaToxins’, alpha-bungarotoxin was grafted onto a large scaffold protein via two peptide bonds that connect alpha-bungarotoxin to a scaffold according to FIG. 2 to build a rigid MegaToxin. The 95 kDa MegaToxin described here is a chimeric polypeptide concatenated from parts of the toxin and parts of a scaffold protein connected according to FIGS. 2 and 7. Here, the toxin used is the alpha-bungarotoxin (BgTX; binding cholinergic receptors) as depicted in SEQ ID NO:3 (PDB 4UY2). The scaffold protein was inserted in the β-turn connecting β-strand 2 and β-strand 3 of the alpha-bungarotoxin. The scaffold protein used was YgjK, a 86 kDa periplasmic protein of E. coli (PDB 3W7S, SEQ ID NO: 5). To create Mt_{BgTx}^{c2YgjK} (SEQ ID NO: 17-20) variants, all parts were connected to each other from the amino to the carboxy terminus in the next given order by peptide bonds: the N-terminus until β-strand 2 of the bungarotoxin (1-17 of SEQ ID NO:3), a peptide linker of one or two amino acids with random composition, the C-terminal part of YgjK (residues 106-760 of SEQ ID NO: 5), a short peptide linker (SEQ ID NO: 10) connecting the C-terminus and the N-terminus of YgjK to produce a circular permutant of the scaffold protein, the N-terminal part of YgjK (residues 1-100 of SEQ ID NO:5), a peptide linker of one or two amino acids with random composition, the C-terminal part from β-strand 3 till end of the bungarotoxin (20-73 of SEQ ID NO: 3), 6×His tag and EPEA tag (U.S. Pat. No. 9,518,084 B2)

To demonstrate that MegaToxin Mt_{BgTx}^{c2YgjK} (SEQ ID NO: 17-20) variants can be expressed as a well-folded and functional proteins, we displayed these MegaToxins on the surface of yeast (Boder, 1997) and examined the specific binding of anti-bungarotoxin polyclonal antibodies to yeast cells displaying this MegaToxin by flow cytometry. In order to display the Mt_{BgTx}^{c2YgjK} (SEQ ID NO: 17-20) on yeast, we used standard methods to construct an open reading frame that encodes the MegaToxin in fusion to a number of accessory peptides and proteins (SEQ ID NO:32-35): the appS4 leader sequence that directs extracellular secretion in yeast (Rakestraw, 2009), the MegaToxin Mt_{BgTx}^{c2YgjK}, a flexible peptide linker, the Aga2p the adhesion subunit of the yeast agglutinin protein Aga2p which attaches to the yeast cell wall through disulfide bonds to Aga1p protein, an acyl carrier protein for the orthogonal fluorescent staining of the displayed fusion protein (Johnsson, 2005) followed by the cMyc Tag. This open reading frame was put under the transcriptional control of galactose-inducible GAL1/10 promotor into a variant of the pNACP vector (Uchariski, 2019) and introduced into yeast strain EBY100. Eighty randomly picked EBY100 yeast clones, bearing this plasmid (with random codons in the linker region), were grown and induced overnight in a galactose-rich medium to trigger the expression and secretion of the MegaToxin-Aga2p-ACP fusion. The expression of MegaToxin Mt_{BgTx}^{c2YgjK} on the surface of yeast is induced by changing growing conditions from glucose-rich to galactose-rich media. The induced EBY100 yeast cells were incubated with anti-bungarotoxin polyclonal antibodies (AgroBio Cat NO. ACPBU103). After washing, the cells were stained with anti-rabbit-FITC (BD Pharmingen Cat NO 554020). When analysing by flow cytometry, we observed a clear shift in the FITC-fluorescence level for many clones indicating the presence of bungarotoxin. Six representatives are shown in FIG. 18A. In contrast, yeast cells expressing Mb_{Nb207}^{cYgjK} (CA12755, a MegaBody™ wherein a Nanobody is grafted on the YgjK scaffold, see also WO2019/086548A1) and stained as described above, showed no shift in the FITC-fluorescence level. The control sample (anti-FITC control) which was stained only with anti-rabbit-FITC to see the background staining of FITC did not show any shift in the FITC-fluorescence level (FIG. 18A). Individual clones were sequence analysed. An example of amino acid (AA) sequences found in the linkers connecting toxin to scaffold can be seen in FIG. 18B.

To prove that these MegaToxins are functional, we incubated clones with the GABA_{A}R β3 homopentamer. The GABA_{A}R β3 construct carries a 1D4-tag and can be detected with the anti-1D4 mAb. After incubation with GABA_{A}R β3, cells were washed and incubated with the anti-1D4 mAb (Sigma Cat NO. 5403) after which they were stained with a goat anti-mouse-FITC (eBioscience Cat NO. 11-4011-85).

Flow cytometric analysis confirmed that GABA_{A}R β3 binds more specific to yeast cells expressing the MegaToxin Mt_{BgTx}^{c2YgjK} then to the irrelevant clone MegaBody Mb_{Nb207}^{cYgjK} (CA12755). When Mt_{BgTx}^{c2YgjK} clones were only stained with anti-1D4 and anti-mouse no shift in the FITC-fluorescence was seen (FIGS. 19A-19D). We conclude from these experiments that the MegaToxin Mt_{BgTx}^{c2YgjK} can be expressed as a functional chimeric fusion protein on the surface of yeast and that the MegaToxin can bind its target.

### Example 8: Design and Generation of a 50 kDa Fusion Protein Built from a c7HopQ Scaffold Inserted into the 8-Strand β2-β3-Connecting β-Turn of Micrurotoxin1 (MmTX1)

As a next example of obtaining rigid fusion proteins ‘MegaToxins’, micrurotoxin1 was grafted onto a large scaffold protein via two peptide bonds that connect micrurotoxin1 to a scaffold according to FIG. 2 to build a rigid MegaToxin. The 50 kDa MegaToxin described here is a chimeric polypeptide concatenated from parts of the toxin and parts of a scaffold protein connected according to FIGS. 2 and 8. Here, the toxin used is the micrurotoxin1 (binding the GAB_{A}A receptor(s)) as depicted in SEQ ID NO:11 (a structural homologue of bungarotoxin PDB 4UY2). The scaffold protein was inserted in the β-turn connecting β-strand 2 and β-strand 3 of the micrurotoxin1. The scaffold protein is an adhesin domain of Helicobacter pylori strain G27 (PDB: 5LP2; SEQ ID NO:16) called HopQ (Javaheri et al, 2016). The N- and C-terminus of HopQ was connected, after a truncation of 7 amino acids in the circular permutation region (called c7HopQ). This truncated fusion creates a circularly permutated variant of HopQ, called c7HopQ, wherein a cleavage within the amino acid sequence was made somewhere else in its sequence (i.e. in a position corresponding to an accessible site in an exposed region of said scaffold protein). Mt_{MmTX1}^{c7HopQ} (SEQ ID NO:21) was generated, where all parts were connected as follows: the N-terminus until β-strand 2 of the micrurotoxin1 (1-18 of SEQ ID NO:11), a C-terminal part of HopQ (residues 192-411 of SEQ ID NO: 16), an N-terminal part of HopQ (residues 18-184 of SEQ ID NO:16), the C-terminal part from β-strand 3 till end of the micrurotoxin1 (21-64 of SEQ ID NO:11), 6×His tag and EPEA tag.

We set out to express the 50 kDa fusion protein in the periplasm of E. coli. In order to express MegaToxin Mt_{MmTX1}^{c7HopQ} in the periplasm of E. coli, we used standard methods to construct a vector that allowed the expression of micrurotoxin1 MegaToxins: scaffolds can be inserted into the β-turn connecting β-strand 2 (β2) and β-strand 3 (β3) of micrurotoxin1. The vector is a derivative of pMESy4 (Pardon et al., 2014) and contains an open reading frame that encodes the following polypeptides: the pelB leader sequence that directs the secretion of the MegaToxin to the periplasm of E. coli, the N-terminus until β-strand β2 of the micrurotoxin1, the circularly permutated variant of HopQ (c7HopQ), the C-terminus from β-strand β3 of the micrurotoxin1, the 6×His tag and the EPEA tag followed by the Amber stop codon.

Independent Mt_{MmTX1}^{c7HopQ} clones were expressed in the periplasm of E. coli in small scale according to Pardon et al. (2014), next they were purified on Ni beads according to standard procedures and analysed on SDS-PAGE by Coomassie blue staining (FIG. 20A). Two clones, called MP1583_C9 and MP1583_A8, were purified at larger scale and a sample was subjected to SDS-PAGE analysis (FIG. 20B), and in parallel also transferred to a nitrocellulose membrane, which was blocked with 4% skimmed milk and analysed by Western blot (FIG. 20C). Expression of recombinant Mt_{MmTX1}^{c7HopQ} was detected by using the biotinylated anti-EPEA (Life Technologies Cat. Nr. 7103252100) as the primary antibody and a streptavidin-alkaline phosphatase conjugate (Promega, V5591) in combination with NBT and BCIP to develop the blot. The detection of bands with the appropriate molecular weight (approx. 50 kDa for the Mt_{MmTX1}^{c7HopQ}) confirms expression of the Mt_{MmTX1}^{c7HopQ} fusion protein. Different clones were sequence analysed. Sequences of the linkers connecting MmTX1 to the c7HopQ scaffold are shown in FIG. 20D.

### Example 9: Design and Generation of a 94 kDa Fusion Protein Built from a c1YgjK Scaffold Inserted into the β-Strand β2-β3-Connecting β-Turn of Micrurotoxin1 (MmTX1)

As a next example of obtaining rigid fusion proteins ‘MegaToxins’, micrurotoxin1 was differently grafted onto a large scaffold protein via two peptide bonds that connect micrurotoxin1 to a scaffold according to FIG. 2 to build a rigid MegaToxin. The 94 kDa MegaToxin described here is a chimeric polypeptide concatenated from parts of the toxin and parts of a scaffold protein connected according to FIGS. 2 and 9. The toxin used here is the micrurotoxin1 as depicted in SEQ ID NO:11. The scaffold protein was inserted in the β-turn connecting β-strand 2 and β-strand 3 of the micrurotoxin1. The scaffold protein used was YgjK, a 86 kDa periplasmic protein of E. coli (PDB 3W7S, SEQ ID NO: 5), as in Example 4, but with a different circular permutation variant (c1Ygjk). To create Mt_{MmTX1}^{c1YgjK} variants all parts were connected to each other from the amino to the carboxy terminus in the next given order by peptide bonds (SEQ ID NO:23-26): the N-terminus until β-strand 2 of the micrurotoxin1 (1-18 of SEQ ID NO:11), a peptide linker of one AA with random composition or of 2 AA with one AA with random composition, the C-terminal part of YgjK (residues 464-760 or 465-760 of SEQ ID NO: 5), a short peptide linker (SEQ ID NO: 10) connecting the C-terminus and the N-terminus of YgjK to produce a circular permutant of the scaffold protein, the N-terminal part of YgjK (residues 1-459 or 1-460 of SEQ ID NO:5), a peptide linker of one AA with random composition or of 2 AA with one AA with random composition, the C-terminal part from β-strand 3 till end of the micrurotoxin1 (21-64 of SEQ ID NO:11), 6×His tag and EPEA tag.

We set out to express the 94 kDa fusion protein in the periplasm of E. coli. In order to express MegaToxin Mt_{MmTX1}^{c1YgjK} in the periplasm of E. coli, we used standard methods to construct a vector that allowed the expression of micrurotoxin1 MegaToxins: scaffolds can be inserted into the β-turn connecting β-strand 2 (β2) and β-strand 3 (β3) of micrurotoxin1. The vector is a derivative of pMESy4 (Pardon et al., 2014) and contains an open reading frame that encodes the following polypeptides: the pelB leader sequence that directs the secretion of the MegaToxin to the periplasm of E. coli, the N-terminus until β-strand β2 of micrurotoxin1, the circularly permutated variant of YgjK (c1YgjK), the C-terminus from β-strand β3 of the micrurotoxin1, the 6×His tag and the EPEA tag followed by the Amber stop codon.

Independent Mt_{MmTX1}^{c1YgjK} clones were expressed in the periplasm of E. coli in small scale according to Pardon et al. (2014), next they were purified on Ni beads according to standard procedures and analysed on SDS-PAGE by Coomassie blue staining. In many clones, a very abundant protein band with a Molecular weight of around 100 kDa could be detected, corresponding to the expected size for the MegaToxins (FIG. 21A). Three clones, MP1639_D3, MP1639_F4, and MP1639_A9, were analysed by SDS-PAGE analysis (FIG. 21B), and in parallel transferred to a nitrocellulose membrane, which was blocked with 4% skimmed milk and analysed by Western blot (FIG. 21C). Expression of recombinant Mt_{MmTX1}^{c1YgjK} was detected by using the biotinylated anti-EPEA (Life Technologies Cat. Nr. 7103252100) as the primary antibody and a streptavidin-alkaline phosphatase conjugate (Promega, V5591) in combination with NBT and BCIP to develop the blot. The detection of bands with the appropriate molecular weight (approximately 94 kDa for the Mt_{MmTX1}^{c1YgjK}) confirms expression of the Mt_{MmTX1}^{c1YgjK} fusion protein. Sequences of the linkers connecting MmTX1 to the c1YgjK scaffold are shown in FIG. 20D.

### Example 10: Design and Generation of a 62 kDa Fusion Protein Built from a c7HopQ Scaffold Inserted into the β-Turn of 2 β-Strands of Sticholysin

As another example of obtaining rigid fusion proteins ‘MegaToxins’, SticholysinII (StII) was grafted onto a large scaffold protein via two peptide bonds that connect Sticholysin to a scaffold according to FIG. 10 to build a rigid MegaToxin. The 62 kDa MegaToxin described here is a chimeric polypeptide concatenated from parts of the toxin and parts of a scaffold protein connected according to FIGS. 10 and 11. Here, the toxin used is Sticholysin II (forming oligomeric aqueous pores in membranes; Garcia et al. 2012) as depicted in SEQ ID NO: 27 (PDB1O72)). The scaffold protein was inserted in the β-turn connecting 2 β-strands of the Sticholysin II. The scaffold protein is an adhesin domain of Helicobacter pylori strain G27 (PDB: 5LP2; SEQ ID NO:16) called HopQ (Javaheri et al, 2016). The N- and C-terminus of HopQ was connected, although after a truncation of 7 amino acids in the circular permutation region (called c7HopQ) which otherwise appeared as a loop never fully visible in electron density of crystal structures. This truncated fusion creates a circularly permutated variant of HopQ, called c7HopQ, wherein a cleavage within the amino acid sequence was made somewhere else in its sequence. A low free energy Mt_{StII}^{c7HopQ} (SEQ ID NO:28) was generated, where all parts were connected as follows: the N-terminus until a β-strand of the Sticholysin II (1-91 of SEQ ID NO: 27), a C-terminal part of HopQ (residues 192-411 of SEQ ID NO: 16), an N-terminal part of HopQ (residues 18-184 of SEQ ID NO:16), the C-terminal part from the β-strand following the β-turn till the end of the Sticholysin II (94-175 of SEQ ID NO:27), 6×His tag and EPEA tag.

We set out to express the 62 kDa fusion protein in the periplasm of E. coli. In order to express MegaToxin Mt_{StII}^{c7HopQ} in the periplasm of E. coli, we used standard methods to construct a vector that allowed the expression of Sticholysin MegaToxins: scaffolds can be inserted into the β-turn connecting β-strand 2 (β2) and β-strand 3 (β3) of Sticholysin. The vector is a derivative of pMESy4 (Pardon et al., 2014) and contains an open reading frame that encodes the following polypeptides: the DsbA leader sequence that directs the secretion of the MegaToxin to the periplasm of E. coli, the N-terminus until β-strand β2 of the Sticholysin, the circularly permutated variant of HopQ (c7HopQ), the C-terminus from β-strand β3 of the Sticholysin, the 6×His tag and the EPEA tag followed by the Amber stop codon.

### Example 11: Design and Generation of a 71 kDa Fusion Protein Built from a c7HopQ Scaffold Inserted into the β-Turn Connecting 2β-Strands of Ricin a Chain (RTA)

As a next example of obtaining rigid fusion proteins ‘MegaToxins’, Ricin A chain fragment 36-302 was grafted onto a large scaffold protein via two peptide bonds that connect Ricin A fragment to a scaffold according to FIG. 10 to build a rigid MegaToxin. The 71 kDa MegaToxin described here is a chimeric polypeptide concatenated from parts of the toxin and parts of a scaffold protein connected according to FIGS. 10 and 12. Here, the toxin used is the Ricin A chain (which enzymatically depurinates a key adenine residue in 28 S rRNA) as depicted in SEQ ID NO:30 (PDB 5J56). The scaffold protein was inserted in the β-turn connecting 2 β-strands of the ricin A chain. The scaffold protein c7HopQ to generate Mt_{RTA36-302}^{c7HopQ} (SEQ ID NO:31) by connection of all parts as follows: the N-terminus until a β-strand of the ricin A chain (1-64 of SEQ ID NO:30), a C-terminal part of HopQ (residues 193-411 of SEQ ID NO: 16), an N-terminal part of HopQ (residues 18-185 of SEQ ID NO:16), the C-terminal part from β-strand till end of the Ricin A chain (67-267 of SEQ ID NO:30), 6×His tag and EPEA tag.

We set out to express the 71 kDa fusion protein in the periplasm of E. coli. In order to express MegaToxin Mt_{RTA}^{c7HopQ} in the periplasm of E. coli, we used standard methods to construct a vector that allowed the expression ricin A chain MegaToxins: scaffolds can be inserted into the β-turn connecting β-strands of ricin A chain. The vector is a derivative of pMESy4 (Pardon et al., 2014) and contains an open reading frame that encodes the following polypeptides: the pelB leader sequence that directs the secretion of the MegaToxin to the periplasm of E. coli, the N-terminus until a β-strand (before the β-turn of insertion) of ricin A chain, the circularly permutated variant of HopQ (c7HopQ), the C-terminus from β-strand following the the β-turn of the ricin A chain, the 6×His tag and the EPEA tag followed by the Amber stop codon.

Independent Mt_{RTA}^{c7HopQ} clones were expressed in the periplasm of E. coli in small scale according to Pardon et al. (2014), next they were purified on Ni beads according to standard procedures and analysed on SDS-PAGE by Coomassie blue staining (FIG. 22A). No MegaToxin expression could be identified from the gel. Next, a small scale affinity purification on the periplasmic extracts of clones expressing Mt_{RTA}^{c7HopQ} was performed using a VHH F5 (SEQ ID NO: 36; PDB:4Z9K), which is a Nanobody specific for the Ricin A chain (Rudolph et al. 2016) The VHH F5 carrying a strep-tag was mixed with the periplasmic extract of Mt_{RTA}^{c7HopQ} clones. Purification of the ricin A chain-VHH complex was done according to the manufacturer's procedures. Following SDS-PAGE, proteins were transferred to a membrane, which was blocked with 4% skimmed milk and analysed by Western blot (FIG. 22B). Expression of recombinant Mt_{RTA}^{c7HopQ} was detected by using the biotinylated anti-EPEA (Life Technologies Cat. Nr. 7103252100) as the primary antibody and a streptavidin-alkaline phosphatase conjugate (Promega, V5591) in combination with NBT and BCIP to develop the blot. The detection of a faint bands with the appropriate molecular weight (approximately 71 kDa for the Mt_{RTA}^{c7HopQ}) confirms expression of the Mt_{RTA}^{c7HopQ} fusion protein. Bands of around 35 kDa were detected on the Western blot as well indicating a cleavage product of the MegaToxin, so further optimalization may be needed.

### Example 12: Design and Generation of a 95 kDa Fusion Protein Built from a c1YgjK Scaffold Inserted into the β-Turn of 2β-Strands of Ts1 Toxin (Ts1)

As a next example of obtaining rigid fusion proteins ‘MegaToxins’, Ts1 toxin was grafted onto a large scaffold protein via two peptide bonds that connect Ts1 toxin to a scaffold according to FIG. 10 to build a rigid MegaToxin. The 95 kDa MegaToxin described here is a chimeric polypeptide concatenated from parts of the toxin and parts of a scaffold protein connected according to FIGS. 10 and 13. The toxin used here is the Ts1 toxin (acts on Voltage-gated Na⁺ channels of insects and mammals) as depicted in SEQ ID NO:37 (PDB 1B7D). The scaffold protein was inserted in the β-turn connecting β-strand 2 and β-strand 3 of the Ts1 toxin (Shenkarev et al. 2019). The scaffold protein used was YgjK. To create Mt_{TS1}^{c1YgjK} variants all parts were connected to each other from the amino to the carboxy terminus in the next given order by peptide bonds (SEQ ID NO:38): the N-terminus until β-strand 2 of the Ts1 (1-37 of SEQ ID NO:37), a peptide linker of one AA with random composition, the C-terminal part of YgjK (residues 464-760 of SEQ ID NO: 5), a short peptide linker (SEQ ID NO: 10) connecting the C-terminus and the N-terminus of YgjK to produce a circular permutant of the scaffold protein, the N-terminal part of YgjK (residues 1-459 of SEQ ID NO:5), a peptide linker of one AA with random composition, the C-terminal part from β-strand 3 till end of the Ts1 toxin (40-61 of SEQ ID NO:37), 6×His tag and EPEA tag.

We set out to express the 95 kDa fusion protein in the periplasm of E. coli. In order to express MegaToxin Mt_{TS1}^{c1YgjK} in the periplasm of E. coli, we used standard methods to construct a vector that allowed the expression of micrurotoxin1 MegaToxins: scaffolds can be inserted into the β-turn connecting β-strand 2 (β2) and β-strand 3 (β3) of Ts1 toxin. The vector is a derivative of pMESy4 (Pardon et al., 2014) and contains an open reading frame that encodes the following polypeptides: the pelB leader sequence that directs the secretion of the MegaToxin to the periplasm of E. coli, the N-terminus until β-strand β2 of Ts1 toxin, the circularly permutated variant of YgjK (c1YgjK), the C-terminus from β-strand β3 of the Ts1 toxin, the 6×His tag and the EPEA tag followed by the Amber stop codon.

Sequence listing

>SEQ ID NO: 1: alpha-cobratoxin (PDB 1YI5)
>SEQ ID NO: 2: Mt_{alpha-cobratoxin}^{c7HopQ}
(Alpha-cobratoxin sequences in bold, C to N connection of HopQ is double underlined,
HopQ sequences in normal text, X is a short peptide linker of 1 AA and random compo-
sition, 6xHis & EPEA tags are underlined with a dotted line)
IRCFITPDITSKDCXKTTTSVIDTTNDAQNLLTQAQTIVNTLKDYCPILIAKSSSSNGGTNNANTPSWQTAGGGKNSCAT
FGAEFSAASDMINNAQKIVQETQQLSANQPKNITQPHNLNLNSPSSLTALAQKMLKNAQSQAEILKLANQVESDFNK
LSSGHLKDYIGKCDASAISSANMTMQNQKNNWGNGCAGVEETQSLLKTSAADFNNQTPQINQAQNLANTLIQELG
NNTYEQLSRLLTNDNGTNSKTSAQAINQAVNNLNERAKTLAGGTTNSPAYQATLLALRSVLGLWNSMGYAVICGGYT
KSPGENNQKDFHYTDENGNGTTINCGGSTNSNGTHSYNGTNTLKADKNVSLSIEQYEKIHEAYQILSKALKQAGLAPL

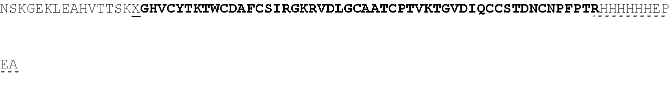

>SEQ ID NO: 3: alpha-bungarotoxin (PDB 4UY2)
>SEQ ID NO: 4: Mt_{alpha-bungarotoxin}^{c7HopQ}
(Alpha-bungarotoxin sequences in bold, C to N connection of HopQ is double underlined,
HopQ sequences in normal text, X is a short peptide linker of 1 AA and random compo-
sition, 6xHis & EPEA tags are underlined with a dotted line)
IVCHTTATSPISAVTCPXKTTTSVIDTTNDAQNLLTQAQTIVNTLKDYCPILIAKSSSSNGGTNNANTPSWQTAGGGKN
SCATFGAEFSAASDMINNAQKIVQETQQLSANQPKNITQPHNLNLNSPSSLTALAQKMLKNAQSQAEILKLANQVES
DFNKLSSGHLKDYIGKCDASAISSANMTMQNQKNNWGNGCAGVEETQSLLKTSAADFNNQTPQINQAQNLANTLI
QELGNNTYEQLSRLLTNDNGTNSKTSAQAINQAVNNLNERAKTLAGGTTNSPAYQATLLALRSVLGLWNSMGYAVIC
GGYTKSPGENNQKDFHYTDENGNGTTINCGGSTNSNGTHSYNGTNTLKADKNVSLSIEQYEKIHEAYQILSKALKQAG

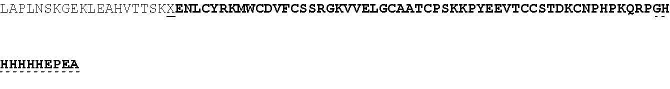

>SEQ ID NO: 5: E.coli Ygjk protein (PDB 3W7S)
>SEQ ID NO: 6: Mt_{Alpha-cobratoxin}^{c2YgjkQ} randomlinkers
(Alpha-cobratoxin sequences in bold, circular permutation linker in italics, Ygjk
sequences in normal text, X is a short peptide linker of 1 AA and random composition,
6xHis & EPEA tags are underlined with a dotted line)
IRCFITPDITSKDCXQVEMTLRFATPRTSLLETKITSNKPLDLVWDGELLEKLEAKEGKPLSDKTIAGEYPDYQRKISATRD
GLKVTFGKVRATWDLLTSGESEYQVHKSLPVQTEINGNRFTSKAHINGSTTLYTTYSHLLTAQEVSKEQMQIRDILARP
AFYLTASQQRWEEYLKKGLTNPDATPEQTRVAVKAIETLNGNWRSPGGAVKFNTVTPSVTGRWFSGNQTWPWDT
WKQAFAMAHFNPDIAKENIRAVFSWQIQPGDSVRPQDVGFVPDLIAWNLSPERGGDGGNWNERNTKPSLAAWSV
MEVYNVTQDKTWVAEMYPKLVAYHDWWLRNRDHNGNGVPEYGATRDKAHNTESGEMLFTVKKGDKEETQSGL
NNYARVVEKGQYDSLEIPAQVAASWESGRDDAAVFGFIDKEQLDKYVANGGKRSDWTVKFAENRSQDGTLLGYSLL
QESVDQASYMYSDNHYLAEMATILGKPEEAKRYRQLAQQLADYINTCMFDPTTQFYYDVRIEDKPLANGCAGKPIVE
RGKGPEGWSPLFNGAATQANADAVVKVMLDPKEFNTFVPLGTAALTNPAFGADIYWRGRVWVDQFWFGLKGME
RYGYRDDALKLADTFFRHAKGLTADGPIQENYNPLTGAQQGAPNFSWSAAHLYMLYNDFFRKQASGGGSGGGGSG
GGGSGNADNYKNVINRTGAPQYMKDYDYDDHQRFNPFFDLGAWHGHLLPDGPNTMGGFPGVALLTEEYINFMAS
NFDRLTVWQDGKKVDFTLEAYSIPGALVQKLXGHVCYTKTWCDAFCSIRGKRVDLGCAATCPTVKTGVDIQCCSTD

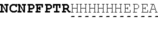

>SEQ ID NO: 7: Mt_{Alpha-cobratoxin}^{c2YgjkQ} randomlinkers
(Alpha-cobratoxin sequences in bold, circular permutation linker in italics, Ygjk
sequences in normal text, X is a short peptide linker of 1 AA and random composition,
XX is a short peptide linker of 2 AA and random composition, 6xHis & EPEA tags are
underlined with a dotted line)
IRCFITPDITSKDCXQVEMTLRFATPRTSLLETKITSNKPLDLVWDGELLEKLEAKEGKPLSDKTIAGEYPDYQRKISATRD
GLKVTFGKVRATWDLLTSGESEYQVHKSLPVQTEINGNRFTSKAHINGSTTLYTTYSHLLTAQEVSKEQMQIRDILARP
AFYLTASQQRWEEYLKKGLTNPDATPEQTRVAVKAIETLNGNWRSPGGAVKFNTVTPSVTGRWFSGNQTWPWDT
WKQAFAMAHFNPDIAKENIRAVFSWQIQPGDSVRPQDVGFVPDLIAWNLSPERGGDGGNWNERNTKPSLAAWSV
MEVYNVTQDKTWVAEMYPKLVAYHDWWLRNRDHNGNGVPEYGATRDKAHNTESGEMLFTVKKGDKEETQSGL
NNYARVVEKGQYDSLEIPAQVAASWESGRDDAAVFGFIDKEQLDKYVANGGKRSDWTVKFAENRSQDGTLLGYSLL
QESVDQASYMYSDNHYLAEMATILGKPEEAKRYRQLAQQLADYINTCMFDPTTQFYYDVRIEDKPLANGCAGKPIVE
RGKGPEGWSPLFNGAATQANADAVVKVMLDPKEFNTFVPLGTAALTNPAFGADIYWRGRVWVDQFWFGLKGME
RYGYRDDALKLADTFFRHAKGLTADGPIQENYNPLTGAQQGAPNFSWSAAHLYMLYNDFFRKQASGGGSGGGGSG
GGGSGNADNYKNVINRTGAPQYMKDYDYDDHQRFNPFFDLGAWHGHLLPDGPNTMGGFPGVALLTEEYINFMAS
NFDRLTVWQDGKKVDFTLEAYSIPGALVQKLXXGHVCYTKTWCDAFCSIRGKRVDLGCAATCPTVKTGVDIQCCST

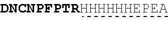

>SEQ ID NO: 8: Mt_{Alpha-cobratoxin}^{c2YgjkQ} randomlinkers
(Alpha-cobratoxin sequences in bold, circular permutation linker in italics,
Ygjk sequences in normal text, X is a short peptide linker of 1 AA and random
composition, XX is a short peptide linker of 2 AA and random composition, 6xHis &
EPEA tags are underlined with a dotted line)
IRCFITPDITSKDCXXQVEMTLRFATPRTSLLETKITSNKPLDLVWDGELLEKLEAKEGKPLSDKTIAGEYPDYQRKISATR
DGLKVTFGKVRATWDLLTSGESEYQVHKSLPVQTEINGNRFTSKAHINGSTTLYTTYSHLLTAQEVSKEQMQIRDILAR
PAFYLTASQQRWEEYLKKGLTNPDATPEQTRVAVKAIETLNGNWRSPGGAVKFNTVTPSVTGRWFSGNQTWPWDT
WKQAFAMAHFNPDIAKENIRAVFSWQIQPGDSVRPQDVGFVPDLIAWNLSPERGGDGGNWNERNTKPSLAAWSV
MEVYNVTQDKTWVAEMYPKLVAYHDWWLRNRDHNGNGVPEYGATRDKAHNTESGEMLFTVKKGDKEETQSGL
NNYARVVEKGQYDSLEIPAQVAASWESGRDDAAVFGFIDKEQLDKYVANGGKRSDWTVKFAENRSQDGTLLGYSLL
QESVDQASYMYSDNHYLAEMATILGKPEEAKRYRQLAQQLADYINTCMFDPTTQFYYDVRIEDKPLANGCAGKPIVE
RGKGPEGWSPLFNGAATQANADAVVKVMLDPKEFNTFVPLGTAALTNPAFGADIYWRGRVWVDQFWFGLKGME
RYGYRDDALKLADTFFRHAKGLTADGPIQENYNPLTGAQQGAPNFSWSAAHLYMLYNDFFRKQASGGGSGGGGSG
GGGSGNADNYKNVINRTGAPQYMKDYDYDDHQRFNPFFDLGAWHGHLLPDGPNTMGGFPGVALLTEEYINFMAS
NFDRLTVWQDGKKVDFTLEAYSIPGALVQKLXGHVCYTKTWCDAFCSIRGKRVDLGCAATCPTVKTGVDIQCCSTD

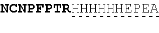

>SEQ ID NO: 9: Mt_{Alpha-cobratoxin}^{c2YgjkQ} randomlinkers
IRCFITPDITSKDCXXQVEMTLRFATPRTSLLETKITSNKPLDLVWDGELLEKLEAKEGKPLSDKTIAGEYPDYQRKISATR
DGLKVTFGKVRATWDLLTSGESEYQVHKSLPVQTEINGNRFTSKAHINGSTTLYTTYSHLLTAQEVSKEQMQIRDILAR
PAFYLTASQQRWEEYLKKGLTNPDATPEQTRVAVKAIETLNGNWRSPGGAVKFNTVTPSVTGRWFSGNQTWPWDT
WKQAFAMAHFNPDIAKENIRAVFSWQIQPGDSVRPQDVGFVPDLIAWNLSPERGGDGGNWNERNTKPSLAAWSV
MEVYNVTQDKTWVAEMYPKLVAYHDWWLRNRDHNGNGVPEYGATRDKAHNTESGEMLFTVKKGDKEETQSGL
NNYARVVEKGQYDSLEIPAQVAASWESGRDDAAVFGFIDKEQLDKYVANGGKRSDWTVKFAENRSQDGTLLGYSLL
QESVDQASYMYSDNHYLAEMATILGKPEEAKRYRQLAQQLADYINTCMFDPTTQFYYDVRIEDKPLANGCAGKPIVE
RGKGPEGWSPLFNGAATQANADAVVKVMLDPKEFNTFVPLGTAALTNPAFGADIYWRGRVWVDQFWFGLKGME
RYGYRDDALKLADTFFRHAKGLTADGPIQENYNPLTGAQQGAPNFSWSAAHLYMLYNDFFRKQASGGGSGGGGSG
GGGSGNADNYKNVINRTGAPQYMKDYDYDDHQRFNPFFDLGAWHGHLLPDGPNTMGGFPGVALLTEEYINFMAS
NFDRLTVWQDGKKVDFTLEAYSIPGALVQKLXXGHVCYTKTWCDAFCSIRGKRVDLGCAATCPTVKTGVDIQCCST

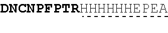

>SEQ ID NO: 10: cYgjk circular permutation linker peptide
>SEQ ID NO: 11: micrurotoxin1
>SEQ ID NO: 12: Mt_{micrurotoxin1}^{c2YgjK} randomlinkers
(micrurotoxin1 sequences in bold, circular permutation linker in italics, Ygjk
sequences in normal text, X is a short peptide linker of 1 AA and random composition,
6xHis & EPEA tags are underlined with a dotted line)
LTCKTCPFTTCPNSESCPXQVEMTLRFATPRTSLLETKITSNKPLDLVWDGELLEKLEAKEGKPLSDKTIAGEYPDYQRKI
SATRDGLKVTFGKVRATWDLLTSGESEYQVHKSLPVQTEINGNRFTSKAHINGSTTLYTTYSHLLTAQEVSKEQMQIRD
ILARPAFYLTASQQRWEEYLKKGLTNPDATPEQTRVAVKAIETLNGNWRSPGGAVKFNTVTPSVTGRWFSGNQTWP
WDTWKQAFAMAHFNPDIAKENIRAVFSWQIQPGDSVRPQDVGFVPDLIAWNLSPERGGDGGNWNERNTKPSLA
AWSVMEVYNVTQDKTWVAEMYPKLVAYHDWWLRNRDHNGNGVPEYGATRDKAHNTESGEMLFTVKKGDKEET
QSGLNNYARVVEKGQYDSLEIPAQVAASWESGRDDAAVFGFIDKEQLDKYVANGGKRSDWTVKFAENRSQDGTLL
GYSLLQESVDQASYMYSDNHYLAEMATILGKPEEAKRYRQLAQQLADYINTCMFDPTTQFYYDVRIEDKPLANGCAG
KPIVERGKGPEGWSPLFNGAATQANADAVVKVMLDPKEFNTFVPLGTAALTNPAFGADIYWRGRVWVDQFWFGL
KGMERYGYRDDALKLADTFFRHAKGLTADGPIQENYNPLTGAQQGAPNFSWSAAHLYMLYNDFFRKQASGGGSGG
GGSGGGGSGNADNYKNVINRTGAPQYMKDYDYDDHQRFNPFFDLGAWHGHLLPDGPNTMGGFPGVALLTEEYIN
FMASNFDRLTVWQDGKKVDFTLEAYSIPGALVQKLXQSICYQRKWEEHRGERIERRCVANCPAFGSHDTSLLCCTRD

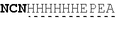

>SEQ ID NO: 13: Mt_{micrurotoxin1}^{c2YgjK} randomlinkers
(micrurotoxin1 sequences in bold, circular permutation linker in italics, Ygjk
sequences in normal text, X is a short peptide linker of 1 AA and random composition,
XX is a short peptide linker of 2 AA and random composition, 6xHis & EPEA tags are
underlined with a dotted line)
LTCKTCPFTTCPNSESCPXQVEMTLRFATPRTSLLETKITSNKPLDLVWDGELLEKLEAKEGKPLSDKTIAGEYPDYQRKI
SATRDGLKVTFGKVRATWDLLTSGESEYQVHKSLPVQTEINGNRFTSKAHINGSTTLYTTYSHLLTAQEVSKEQMQIRD
ILARPAFYLTASQQRWEEYLKKGLTNPDATPEQTRVAVKAIETLNGNWRSPGGAVKFNTVTPSVTGRWFSGNQTWP
WDTWKQAFAMAHFNPDIAKENIRAVFSWQIQPGDSVRPQDVGFVPDLIAWNLSPERGGDGGNWNERNTKPSLA
AWSVMEVYNVTQDKTWVAEMYPKLVAYHDWWLRNRDHNGNGVPEYGATRDKAHNTESGEMLFTVKKGDKEET
QSGLNNYARVVEKGQYDSLEIPAQVAASWESGRDDAAVFGFIDKEQLDKYVANGGKRSDWTVKFAENRSQDGTLL
GYSLLQESVDQASYMYSDNHYLAEMATILGKPEEAKRYRQLAQQLADYINTCMFDPTTQFYYDVRIEDKPLANGCAG
KPIVERGKGPEGWSPLFNGAATQANADAVVKVMLDPKEFNTFVPLGTAALTNPAFGADIYWRGRVWVDQFWFGL
KGMERYGYRDDALKLADTFFRHAKGLTADGPIQENYNPLTGAQQGAPNFSWSAAHLYMLYNDFFRKQASGGGSGG
GGSGGGGSGNADNYKNVINRTGAPQYMKDYDYDDHQRFNPFFDLGAWHGHLLPDGPNTMGGFPGVALLTEEYIN
FMASNFDRLTVWQDGKKVDFTLEAYSIPGALVQKLXXQSICYQRKWEEHRGERIERRCVANCPAFGSHDTSLLCCTR

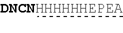

>SEQ ID NO: 14: Mt_{micrurotoxin1}^{c2YgjK} randomlinkers
(micrurotoxin1 sequences in bold, circular permutation linker in italics, Ygjk
sequences in normal text, X is a short peptide linker of 1 AA and random composition,
XX is a short peptide linker of 2 AA and random composition, 6xHis & EPEA tags are
underlined with a dotted line)
LTCKTCPFTTCPNSESCPXXQVEMTLRFATPRTSLLETKITSNKPLDLVWDGELLEKLEAKEGKPLSDKTIAGEYPDYQRK
ISATRDGLKVTFGKVRATWDLLTSGESEYQVHKSLPVQTEINGNRFTSKAHINGSTTLYTTYSHLLTAQEVSKEQMQIR
DILARPAFYLTASQQRWEEYLKKGLTNPDATPEQTRVAVKAIETLNGNWRSPGGAVKFNTVTPSVTGRWFSGNQTW
PWDTWKQAFAMAHFNPDIAKENIRAVFSWQIQPGDSVRPQDVGFVPDLIAWNLSPERGGDGGNWNERNTKPSL
AAWSVMEVYNVTQDKTWVAEMYPKLVAYHDWWLRNRDHNGNGVPEYGATRDKAHNTESGEMLFTVKKGDKEE
TQSGLNNYARVVEKGQYDSLEIPAQVAASWESGRDDAAVFGFIDKEQLDKYVANGGKRSDWTVKFAENRSQDGTLL
GYSLLQESVDQASYMYSDNHYLAEMATILGKPEEAKRYRQLAQQLADYINTCMFDPTTQFYYDVRIEDKPLANGCAG
KPIVERGKGPEGWSPLFNGAATQANADAVVKVMLDPKEFNTFVPLGTAALTNPAFGADIYWRGRVWVDQFWFGL
KGMERYGYRDDALKLADTFFRHAKGLTADGPIQENYNPLTGAQQGAPNFSWSAAHLYMLYNDFFRKQASGGGSGG
GGSGGGGSGNADNYKNVINRTGAPQYMKDYDYDDHQRFNPFFDLGAWHGHLLPDGPNTMGGFPGVALLTEEYIN
FMASNFDRLTVWQDGKKVDFTLEAYSIPGALVQKLXQSICYQRKWEEHRGERIERRCVANCPAFGSHDTSLLCCTRD

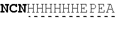

>SEQ ID NO: 15: Mt_{micrurotoxin1}^{c2YgjK} randomlinkers
(micrurotoxin1 sequences in bold, circular permutation linker in italics, Ygjk
sequences in normal text, XX is a short peptide linker of 2 AA and random
composition, 6xHis & EPEA tags are underlined with a dotted line)
LTCKTCPFTTCPNSESCPXXQVEMTLRFATPRTSLLETKITSNKPLDLVWDGELLEKLEAKEGKPLSDKTIAGEYPDYQRK
ISATRDGLKVTFGKVRATWDLLTSGESEYQVHKSLPVQTEINGNRFTSKAHINGSTTLYTTYSHLLTAQEVSKEQMQIR
DILARPAFYLTASQQRWEEYLKKGLTNPDATPEQTRVAVKAIETLNGNWRSPGGAVKFNTVTPSVTGRWFSGNQTW
PWDTWKQAFAMAHFNPDIAKENIRAVFSWQIQPGDSVRPQDVGFVPDLIAWNLSPERGGDGGNWNERNTKPSL
AAWSVMEVYNVTQDKTWVAEMYPKLVAYHDWWLRNRDHNGNGVPEYGATRDKAHNTESGEMLFTVKKGDKEE
TQSGLNNYARVVEKGQYDSLEIPAQVAASWESGRDDAAVFGFIDKEQLDKYVANGGKRSDWTVKFAENRSQDGTLL
GYSLLQESVDQASYMYSDNHYLAEMATILGKPEEAKRYRQLAQQLADYINTCMFDPTTQFYYDVRIEDKPLANGCAG
KPIVERGKGPEGWSPLFNGAATQANADAVVKVMLDPKEFNTFVPLGTAALTNPAFGADIYWRGRVWVDQFWFGL
KGMERYGYRDDALKLADTFFRHAKGLTADGPIQENYNPLTGAQQGAPNFSWSAAHLYMLYNDFFRKQASGGGSGG
GGSGGGGSGNADNYKNVINRTGAPQYMKDYDYDDHQRFNPFFDLGAWHGHLLPDGPNTMGGFPGVALLTEEYIN
FMASNFDRLTVWQDGKKVDFTLEAYSIPGALVQKLXXQSICYQRKWEEHRGERIERRCVANCPAFGSHDTSLLCCTR

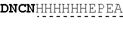

>SEQ ID NO: 16: Helicobacter pylori strain G27 HopQ adhesin domain protein (PDB 5LP2)
MAVQKVKNADKVQKLSDTYEQLSRLLTNDNGTNSKTSAQAINQAVNNLNERAKTLAGGTTNSPAYQATLLALRSVL
GLWNSMGYAVICGGYTKSPGENNQKDFHYTDENGNGTTINCGGSTNSNGTHSYNGTNTLKADKNVSLSIEQYEKIH
EAYQILSKALKQAGLAPLNSKGEKLEAHVTTSKYQQDNQTKTTTSVIDTTNDAQNLLTQAQTIVNTLKDYCPILIAKSSS
SNGGTNNANTPSWQTAGGGKNSCATFGAEFSAASDMINNAQKIVQETQQLSANQPKNITQPHNLNLNSPSSLTAL
AQKMLKNAQSQAEILKLANQVESDFNKLSSGHLKDYIGKCDASAISSANMTMQNQKNNWGNGCAGVEETQSLLKT
SAADFNNQTPQINQAQNLANTLIQELGNNPFRNMGMIASSTTNNGA
>SEQ ID NO: 17-20: Mt_{BgTX}^{c2Ygjk} randomlinkers
(Alpha-bungarotoxin sequences in bold, circular permutation linker in italics, Ygjk
sequences in normal text, X is a short peptide linker of 1 AA and random composition,
6xHis & EPEA tags are underlined with a dotted line)
IVCHTTATSPISAVTCP(X)₁₋₂QVEMTLRFATPRTSLLETKITSNKPLDLVWDGELLEKLEAKEGKPLSDKTIAGEYPDYQR
KISATRDGLKVTFGKVRATWDLLTSGESEYQVHKSLPVQTEINGNRFTSKAHINGSTTLYTTYSHLLTAQEVSKEQMQI
RDILARPAFYLTASQQRWEEYLKKGLTNPDATPEQTRVAVKAIETLNGNWRSPGGAVKFNTVTPSVTGRWFSGNQT
WPWDTWKQAFAMAHFNPDIAKENIRAVFSWQIQPGDSVRPQDVGFVPDLIAWNLSPERGGDGGNWNERNTKPS
LAAWSVMEVYNVTQDKTWVAEMYPKLVAYHDWWLRNRDHNGNGVPEYGATRDKAHNTESGEMLFTVKKGDKE
ETQSGLNNYARVVEKGQYDSLEIPAQVAASWESGRDDAAVFGFIDKEQLDKYVANGGKRSDWTVKFAENRSQDGTL
LGYSLLQESVDQASYMYSDNHYLAEMATILGKPEEAKRYRQLAQQLADYINTCMFDPTTQFYYDVRIEDKPLANGCA
GKPIVERGKGPEGWSPLFNGAATQANADAVVKVMLDPKEFNTFVPLGTAALTNPAFGADIYWRGRVWVDQFWFG
LKGMERYGYRDDALKLADTFFRHAKGLTADGPIQENYNPLTGAQQGAPNFSWSAAHLYMLYNDFFRKQASGGGSG
GGGSGGGGSGNADNYKNVINRTGAPQYMKDYDYDDHQRFNPFFDLGAWHGHLLPDGPNTMGGFPGVALLTEEYI
NFMASNFDRLTVWQDGKKVDFTLEAYSIPGALVQKL(X)₁₋₂ENLCYRKMWCDVFCSSRGKVVELGCAATCPSKKPYE

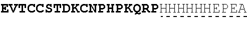

>SEQ ID NO: 21: Mt_{MmTX1}^{c7HopQ}
(micrurotoxin1 sequences in bold, connection of C- and N term is double underlined,
HopQ sequences in normal text, X is a short peptide linker of 1 AA and random
composition, 6xHis & EPEA tags are underlined with a dotted line)
LTCKTCPFTTCPNSESCPXTKTTTSVIDTTNDAQNLLTQAQTIVNTLKDYCPILIAKSSSSNGGTNNANTPSWQTAGGG
KNSCATFGAEFSAASDMINNAQKIVQETQQLSANQPKNITQPHNLNLNSPSSLTALAQKMLKNAQSQAEILKLANQV
ESDFNKLSSGHLKDYIGKCDASAISSANMTMQNQKNNWGNGCAGVEETQSLLKTSAADFNNQTPQINQAQNLANT
LIQELGNNTYEQLSRLLTNDNGTNSKTSAQAINQAVNNLNERAKTLAGGTTNSPAYQATLLALRSVLGLWNSMGYAV
ICGGYTKSPGENNQKDFHYTDENGNGTTINCGGSTNSNGTHSYNGTNTLKADKNVSLSIEQYEKIHEAYQILSKALKQ

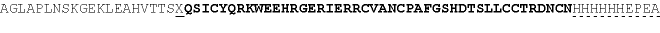

>SEQ ID NO: 22: Mt_{BgTX}^{c7HopQ}_Aga2p_ACP protein sequence
(appS4 leader sequence, MegaToxin Mt_{BgTX}^{c7Hop} depicted in bold, flexible (GGGS)ₙ poly-
peptide linker, Aga2p protein sequence underlined, ACP sequence double underlined,
cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAIVCHTTATSPISAVTCPXKTTTSVIDTTNDAQNLLTQAQTIVNTLKDYCPILIAKSSSSNGGTNNANTPS
WQTAGGGKNSCATFGAEFSAASDMINNAQKIVQETQQLSANQPKNITQPHNINLNSPSSLTALAQKMLKNAQS
QAEILKLANQVESDFNKLSSGHLKDYIGKCDASAISSANMTMQNQKNNWGNGCAGVEETQSLLKTSAADFNNQT
PQINQAQNLANTLIQELGNNTYEQLSRLLTNDNGTNSKTSAQAINQAVNNLNERAKTLAGGTTNSPAYQATLLAL
RSVLGLWNSMGYAVICGGYTKSPGENNQKDFHYTDENGNGTTINCGGSTNSNGTHSYNGTNTLKADKNVSLSIE
QYEKIHEAYQILSKALKQAGLAPLNSKGEKLEAHVTTSKXENLCYRKMWCDVFCSSRGKVVELGCAATCPSKKPYEE
VTCCSTDKCNPHPKQRPGSLGGGSGGGGSGGGGSGGGGSGGGGSGGGGSGGGGSQELTTICEQIPSPTLESTPYSL
STTTILANGKAMQGVFEYYKSVTFVSNCGSHPSTTSKGSPINTQYVFKDNSSTSMSTIEERVKKIIGEQLGVKQEEVTNN
ASFVEDLGADSLDTVELVMALEEEFDTEIPDEEAEKITTVQAAIDYINGHQASEQKLISEEDL
>SEQ ID NO: 23: Mt_{MmTX1}^{c1YgjK} randomlinkers
(micrurotoxin1 sequences in bold, circular permutation linker in italics,
Ygjk sequences in normal text, X is a short peptide linker of 1 AA and random
composition, 6xHis & EPEA tags are underlined with a dotted line)
LTCKTCPFTTCPNSESCPXKEETQSGLNNYARVVEKGQYDSLEIPAQVAASWESGRDDAAVFGFIDKEQLDKYVANG
GKRSDWTVKFAENRSQDGTLLGYSLLQESVDQASYMYSDNHYLAEMATILGKPEEAKRYRQLAQQLADYINTCMFD
PTTQFYYDVRIEDKPLANGCAGKPIVERGKGPEGWSPLFNGAATQANADAVVKVMLDPKEFNTFVPLGTAALTNPAF
GADIYWRGRVWVDQFWFGLKGMERYGYRDDALKLADTFFRHAKGLTADGPIQENYNPLTGAQQGAPNFSWSAA
HLYMLYNDFFRKQASGGGSGGGGSGGGGSGNADNYKNVINRTGAPQYMKDYDYDDHQRFNPFFDLGAWHGHLL
PDGPNTMGGFPGVALLTEEYINFMASNFDRLTVWQDGKKVDFTLEAYSIPGALVQKLTAKDVQVEMTLRFATPRTSL
LETKITSNKPLDLVWDGELLEKLEAKEGKPLSDKTIAGEYPDYQRKISATRDGLKVTFGKVRATWDLLTSGESEYQVHKS
LPVQTEINGNRFTSKAHINGSTTLYTTYSHLLTAQEVSKEQMQIRDILARPAFYLTASQQRWEEYLKKGLTNPDATPEQ
TRVAVKAIETLNGNWRSPGGAVKFNTVTPSVTGRWFSGNQTWPWDTWKQAFAMAHFNPDIAKENIRAVFSWQI
QPGDSVRPQDVGFVPDLIAWNLSPERGGDGGNWNERNTKPSLAAWSVMEVYNVTQDKTWVAEMYPKLVAYHD
WWLRNRDHNGNGVPEYGATRDKAHNTESGEMLFTVKXQSICYQRKWEEHRGERIERRCVANCPAFGSHDTSLLC

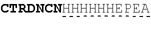

>SEQ ID NO: 24: Mt_{MmTX1}^{c1YgjK} randomlinkers
(micrurotoxin1 sequences in bold, circular permutation linker in italics, Ygjk
sequences in normal text, X is a short peptide linker of 1 AA and random composition,
6xHis & EPEA tags are underlined with a dotted line)
LTCKTCPFTTCPNSESCPXEETQSGLNNYARVVEKGQYDSLEIPAQVAASWESGRDDAAVFGFIDKEQLDKYVANGG
KRSDWTVKFAENRSQDGTLLGYSLLQESVDQASYMYSDNHYLAEMATILGKPEEAKRYRQLAQQLADYINTCMFDPT
TQFYYDVRIEDKPLANGCAGKPIVERGKGPEGWSPLFNGAATQANADAVVKVMLDPKEFNTFVPLGTAALTNPAFG
ADIYWRGRVWVDQFWFGLKGMERYGYRDDALKLADTFFRHAKGLTADGPIQENYNPLTGAQQGAPNFSWSAAHL
YMLYNDFFRKQASGGGSGGGGSGGGGSGNADNYKNVINRTGAPQYMKDYDYDDHQRFNPFFDLGAWHGHLLPD
GPNTMGGFPGVALLTEEYINFMASNFDRLTVWQDGKKVDFTLEAYSIPGALVQKLTAKDVQVEMTLRFATPRTSLLE
TKITSNKPLDLVWDGELLEKLEAKEGKPLSDKTIAGEYPDYQRKISATRDGLKVTFGKVRATWDLLTSGESEYQVHKSLP
VQTEINGNRFTSKAHINGSTTLYTTYSHLLTAQEVSKEQMQIRDILARPAFYLTASQQRWEEYLKKGLTNPDATPEQTR
VAVKAIETLNGNWRSPGGAVKFNTVTPSVTGRWFSGNQTWPWDTWKQAFAMAHFNPDIAKENIRAVFSWQIQP
GDSVRPQDVGFVPDLIAWNLSPERGGDGGNWNERNTKPSLAAWSVMEVYNVTQDKTWVAEMYPKLVAYHDW
WLRNRDHNGNGVPEYGATRDKAHNTESGEMLFTVKXQSICYQRKWEEHRGERIERRCVANCPAFGSHDTSLLCCT

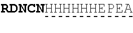

>SEQ ID NO: 25: Mt_{MmTX1}^{c1YgjK} randomlinkers
(micrurotoxin1 sequences in bold in bold, circular permutation linker in italics,
Ygjk sequences in normal text, X is a short peptide linker of 1 AA and random
composition, 6xHis & EPEA tags are underlined with a dotted line)
LTCKTCPFTTCPNSESCPXKEETQSGLNNYARVVEKGQYDSLEIPAQVAASWESGRDDAAVFGFIDKEQLDKYVANG
GKRSDWTVKFAENRSQDGTLLGYSLLQESVDQASYMYSDNHYLAEMATILGKPEEAKRYRQLAQQLADYINTCMFD
PTTQFYYDVRIEDKPLANGCAGKPIVERGKGPEGWSPLFNGAATQANADAVVKVMLDPKEFNTFVPLGTAALTNPAF
GADIYWRGRVWVDQFWFGLKGMERYGYRDDALKLADTFFRHAKGLTADGPIQENYNPLTGAQQGAPNFSWSAA
HLYMLYNDFFRKQASGGGSGGGGSGGGGSGNADNYKNVINRTGAPQYMKDYDYDDHQRFNPFFDLGAWHGHLL
PDGPNTMGGFPGVALLTEEYINFMASNFDRLTVWQDGKKVDFTLEAYSIPGALVQKLTAKDVQVEMTLRFATPRTSL
LETKITSNKPLDLVWDGELLEKLEAKEGKPLSDKTIAGEYPDYQRKISATRDGLKVTFGKVRATWDLLTSGESEYQVHKS
LPVQTEINGNRFTSKAHINGSTTLYTTYSHLLTAQEVSKEQMQIRDILARPAFYLTASQQRWEEYLKKGLTNPDATPEQ
TRVAVKAIETLNGNWRSPGGAVKFNTVTPSVTGRWFSGNQTWPWDTWKQAFAMAHFNPDIAKENIRAVFSWQI
QPGDSVRPQDVGFVPDLIAWNLSPERGGDGGNWNERNTKPSLAAWSVMEVYNVTQDKTWVAEMYPKLVAYHD
WWLRNRDHNGNGVPEYGATRDKAHNTESGEMLFTVXQSICYQRKWEEHRGERIERRCVANCPAFGSHDTSLLCC

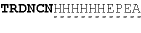

>SEQ ID NO: 26: Mt_{MmTX1}^{c1YgjK} randomlinkers
(micrurotoxin1 sequences in bold, circular permutation linker in italics, Ygjk
sequences in normal text, X is a short peptide linker of 1 AA and random compo-
sition, 6xHis & EPEA tags are underlined with a dotted line)
LTCKTCPFTTCPNSESCPXEETQSGLNNYARVVEKGQYDSLEIPAQVAASWESGRDDAAVFGFIDKEQLDKYVANGG
KRSDWTVKFAENRSQDGTLLGYSLLQESVDQASYMYSDNHYLAEMATILGKPEEAKRYRQLAQQLADYINTCMFDPT
TQFYYDVRIEDKPLANGCAGKPIVERGKGPEGWSPLFNGAATQANADAVVKVMLDPKEFNTFVPLGTAALTNPAFG
ADIYWRGRVWVDQFWFGLKGMERYGYRDDALKLADTFFRHAKGLTADGPIQENYNPLTGAQQGAPNFSWSAAHL
YMLYNDFFRKQASGGGSGGGGSGGGGSGNADNYKNVINRTGAPQYMKDYDYDDHQRFNPFFDLGAWHGHLLPD
GPNTMGGFPGVALLTEEYINFMASNFDRLTVWQDGKKVDFTLEAYSIPGALVQKLTAKDVQVEMTLRFATPRTSLLE
TKITSNKPLDLVWDGELLEKLEAKEGKPLSDKTIAGEYPDYQRKISATRDGLKVTFGKVRATWDLLTSGESEYQVHKSLP
VQTEINGNRFTSKAHINGSTTLYTTYSHLLTAQEVSKEQMQIRDILARPAFYLTASQQRWEEYLKKGLTNPDATPEQTR
VAVKAIETLNGNWRSPGGAVKFNTVTPSVTGRWFSGNQTWPWDTWKQAFAMAHFNPDIAKENIRAVFSWQIQP
GDSVRPQDVGFVPDLIAWNLSPERGGDGGNWNERNTKPSLAAWSVMEVYNVTQDKTWVAEMYPKLVAYHDW
WLRNRDHNGNGVPEYGATRDKAHNTESGEMLFTVXQSICYQRKWEEHRGERIERRCVANCPAFGSHDTSLLCCTR

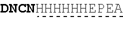

>SEQ ID NO: 27: Sticholysin II (PDB1O72)
>SEQ ID NO: 28: Mt_{StII}^{c7HopQ} randomlinkers
(Sticholysin II sequences in bold, connection of C- and N term is double underlined,
HopQ sequences in normal text, X is a short peptide linker of 1 AA and random compo-
sition, 6xHis & EPEA tags are underlined with a dotted line)
ALAGTIIAGASLTFQVLDKVLEELGKVSRKIAVGIDNESGGTWTALNAYFRSGTTDVILPEFVPNTKALLYSGRKDTG
PVATGAVAAFAYYXTKTTTSVIDTTNDAQNLLTQAQTIVNTLKDYCPILIAKSSSSNGGTNNANTPSWQTAGGGKNS
CATFGAEFSAASDMINNAQKIVQETQQLSANQPKNITQPHNLNLNSPSSLTALAQKMLKNAQSQAEILKLANQVESD
FNKLSSGHLKDYIGKCDASAISSANMTMQNQKNNWGNGCAGVEETQSLLKTSAADFNNQTPQINQAQNLANTLIQ
ELGNNTYEQLSRLLTNDNGTNSKTSAQAINQAVNNLNERAKTLAGGTTNSPAYQATLLALRSVLGLWNSMGYAVICG
GYTKSPGENNQKDFHYTDENGNGTTINCGGSTNSNGTHSYNGTNTLKADKNVSLSIEQYEKIHEAYQILSKALKQAGL
APLNSKGEKLEAHVTTSXSGNTLGVMFSVPFDYNWYSNWWDVKIYSGKRRADQGMYEDLYYGNPYRGDNGWH

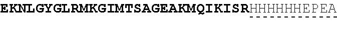

>SEQ ID NO: 29: Mt_{StII}^{c1YgjK} randomlinkers
(Sticholysin II sequences in bold, connection of C- and N term is double underlined,
HopQ sequences in normal text, X is a short peptide linker of 1 AA and random
composition, 6xHis & EPEA tags are underlined with a dotted line)
ALAGTIIAGASLTFQVLDKVLEELGKVSRKIAVGIDNESGGTWTALNAYFRSGTTDVILPEFVPNTKALLYSGRKDTG
PVATGAVAAFAYYXEETQSGLNNYARVVEKGQYDSLEIPAQVAASWESGRDDAAVFGFIDKEQLDKYVANGGKRSD
WTVKFAENRSQDGTLLGYSLLQESVDQASYMYSDNHYLAEMATILGKPEEAKRYRQLAQQLADYINTCMFDPTTQFY
YDVRIEDKPLANGCAGKPIVERGKGPEGWSPLFNGAATQANADAVVKVMLDPKEFNTFVPLGTAALTNPAFGADIY
WRGRVWVDQFWFGLKGMERYGYRDDALKLADTFFRHAKGLTADGPIQENYNPLTGAQQGAPNFSWSAAHLYML
YNDFFRKQASGGGSGGGGSGGGGSGNADNYKNVINRTGAPQYMKDYDYDDHQRFNPFFDLGAWHGHLLPDGPN
TMGGFPGVALLTEEYINFMASNFDRLTVWQDGKKVDFTLEAYSIPGALVQKLTAKDVQVEMTLRFATPRTSLLETKITS
NKPLDLVWDGELLEKLEAKEGKPLSDKTIAGEYPDYQRKISATRDGLKVTFGKVRATWDLLTSGESEYQVHKSLPVQTE
INGNRFTSKAHINGSTTLYTTYSHLLTAQEVSKEQMQIRDILARPAFYLTASQQRWEEYLKKGLTNPDATPEQTRVAVK
AIETLNGNWRSPGGAVKFNTVTPSVTGRWFSGNQTWPWDTWKQAFAMAHFNPDIAKENIRAVFSWQIQPGDSV
RPQDVGFVPDLIAWNLSPERGGDGGNWNERNTKPSLAAWSVMEVYNVTQDKTWVAEMYPKLVAYHDWWLRNR
DHNGNGVPEYGATRDKAHNTESGEMLFTVXSGNTLGVMFSVPFDYNWYSNWWDVKIYSGKRRADQGMYEDLY

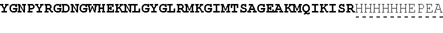

>SEQ ID NO: 30: ricin A chain fragment 36-302 (PDB 5J56)
>SEQ ID NO: 31: Mt_{RTA36-302}^{c7HopQ}
IFPKQYPIINFTTAGATVQSYTNFIRAVRGRLTTGADVRHEIPVLPNRVGLPINQRFILVELSNXKTTTSVIDTTNDAQN
LLTQAQTIVNTLKDYCPILIAKSSSSNGGTNNANTPSWQTAGGGKNSCATFGAEFSAASDMINNAQKIVQETQQLSA
NQPKNITQPHNLNLNSPSSLTALAQKMLKNAQSQAEILKLANQVESDFNKLSSGHLKDYIGKCDASAISSANMTMQN
QKNNWGNGCAGVEETQSLLKTSAADFNNQTPQINQAQNLANTLIQELGNNTYEQLSRLLTNDNGTNSKTSAQAIN
QAVNNLNERAKTLAGGTTNSPAYQATLLALRSVLGLWNSMGYAVICGGYTKSPGENNQKDFHYTDENGNGTTINCG
GSTNSNGTHSYNGTNTLKADKNVSLSIEQYEKIHEAYQILSKALKQAGLAPLNSKGEKLEAHVTTSKXELSVTLALDVTN
AYVVGYRAGNSAYFFHPDNQEDAEAITHLFTDVQNRYTFAFGGNYDRLEQLAGNLRENIELGNGPLEEAISALYYYS
TGGTQLPTLARSFIICIQMISEAARFQYIEGEMRTRIRYNRRSAPDPSVITLENSWGRLSTAIQESNQGAFASPIQLQR

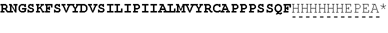

>SEQ ID NO: 32-35: Mt_{BgTx}^{c2YgjK}-Aga2p_ACP protein sequence
(appS4 leader sequence, MegaToxin Mt_{BgTx}^{c2YgjK} depicted in bold, flexible (GGGS)ₙ poly-
peptide linker, Aga2p protein sequence underlined, ACP sequence double underlined,
cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAIVCHTTATSPISAVTCP(X)₁₋₂QVEMTLRFATPRTSLLETKITSNKPLDLVWDGELLEKLEAKEGKPLSDK
TIAGEYPDYQRKISATRDGLKVTFGKVRATWDLLTSGESEYQVHKSLPVQTEINGNRFTSKAHINGSTTLYTTYSHLL
TAQEVSKEQMQIRDILARPAFYLTASQQRWEEYLKKGLTNPDATPEQTRVAVKAIETLNGNWRSPGGAVKFNTVT
PSVTGRWFSGNQTWPWDTWKQAFAMAHFNPDIAKENIRAVFSWQIQPGDSVRPQDVGFVPDLIAWNLSPER
GGDGGNWNERNTKPSLAAWSVMEVYNVTQDKTWVAEMYPKLVAYHDWWLRNRDHNGNGVPEYGATRDKA
HNTESGEMLFTVKKGDKEETQSGLNNYARVVEKGQYDSLEIPAQVAASWESGRDDAAVFGFIDKEQLDKYVANG
GKRSDWTVKFAENRSQDGTLLGYSLLQESVDQASYMYSDNHYLAEMATILGKPEEAKRYRQLAQQLADYINTCM
FDPTTQFYYDVRIEDKPLANGCAGKPIVERGKGPEGWSPLFNGAATQANADAVVKVMLDPKEFNTFVPLGTAALT
NPAFGADIYWRGRVWVDQFWFGLKGMERYGYRDDALKLADTFFRHAKGLTADGPIQENYNPLTGAQQGAPNF
SWSAAHLYMLYNDFFRKQ [symbol] NADNYKNVINRTGAPQYMKDYDYDDHQRFNPFFDLG
AWHGHLLPDGPNTMGGFPGVALLTEEYINFMASNFDRLTVWQDGKKVDFTLEAYSIPGALVQKL(X)₁₋₂ENLCYRK
MWCDVFCSSRGKVVELGCAATCPSKKPYEEVTCCSTDKCNPHPKQRPGSLGGGSGGGGSGGGGSGGGGSGGGG
SGGGGSGGGGSQELTTICEQIPSPTLESTPYSLSTTTILANGKAMQGVFEYYKSVTFVSNCGSHPSTTSKGSPINTQYVF
KDNSSTSMSTIEERVKKIIGEQLGVKQEEVTNNASFVEDLGADSLDTVELVMALEEEFDTEIPDEEAEKITTVGAAIDYIN
GHQASEQKLISEEDL
>SEQ ID NO: 36: VHH F5 (PDB:4Z9K)
QVQLVESGGGIVQPGGSLRLSCAASGFTLDDYAIGWFRQVPGKEREGVACVKDGSTYYADSVKGRFTISRDNGAVYL
QMNSLKPEDTAVYYCASRPCFLGVPLIDFGSWGQGTQVTVSSSAWSHPQFEK
>SEQ ID NO: 37: Ts1 toxin (PDB 1B7D)
>SEQ ID NO: 38: Mt_{Ts1}^{c1YgjK}
(TS1 toxin sequences in bold, circular permutation linker in italics, Ygjk sequences
in normal text, X is a short peptide linker of 1 AA and random composition, 6xHis &
EPEA tags are underlined with a dotted line)
KEGYLMDHEGCKLSCFIRPSGYCGRECGIKKGSSGYCXKEETQSGLNNYARVVEKGQYDSLEIPAQVAASWESGRDD
AAVFGFIDKEQLDKYVANGGKRSDWTVKFAENRSQDGTLLGYSLLQESVDQASYMYSDNHYLAEMATILGKPEEAKR
YRQLAQQLADYINTCMFDPTTQFYYDVRIEDKPLANGCAGKPIVERGKGPEGWSPLFNGAATQANADAVVKVMLDP
KEFNTFVPLGTAALTNPAFGADIYWRGRVWVDQFWFGLKGMERYGYRDDALKLADTFFRHAKGLTADGPIQENYN
PLTGAQQGAPNFSWSAAHLYMLYNDFFRKQASGGGSGGGGSGGGGSGNADNYKNVINRTGAPQYMKDYDYDDH
QRFNPFFDLGAWHGHLLPDGPNTMGGFPGVALLTEEYINFMASNFDRLTVWQDGKKVDFTLEAYSIPGALVQKLTA
KDVQVEMTLRFATPRTSLLETKITSNKPLDLVWDGELLEKLEAKEGKPLSDKTIAGEYPDYQRKISATRDGLKVTFGKVR
ATWDLLTSGESEYQVHKSLPVQTEINGNRFTSKAHINGSTTLYTTYSHLLTAQEVSKEQMQIRDILARPAFYLTASQQR
WEEYLKKGLTNPDATPEQTRVAVKAIETLNGNWRSPGGAVKFNIVTPSVTGRWFSGNQTWPWDTWKQAFAMAH
FNPDIAKENIRAVFSWQIQPGDSVRPQDVGFVPDLIAWNLSPERGGDGGNWNERNTKPSLAAWSVMEVYNVTQD
KTWVAEMYPKLVAYHDWWLRNRDHNGNGVPEYGATRDKAHNTESGEMLFTVXPACYCYGLPNWVKVWDRAT

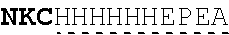

### REFERENCES

Banerjee, A., et al. (2013) Structure of a pore-blocking toxin in complex with a eukaryotic voltage-dependent K(+) channel. eLife 2, e00594 DOI: 10.7554/eLife.00594.
Bliven, S., Prlic, A. (2012). Circular permutation in proteins. PLOS Comput. Biol. 8(3):e1002445.
Boder, E. T., and Wittrup, K. D. (1997). Yeast surface display for screening combinatorial polypeptide libraries. Nat Biotechnol 15, 553-557.
Chao, G., Lau, W. L., Hackel, B. J., Sazinsky, S. L., Lippow, S. M., and Wittrup, K. D. (2006). Isolating and engineering human antibodies using yeast surface display. Nat Protoc 1, 755-768.
Chen et al., 2018. Animal protein toxins: origins and therapeutic applications. Biophys Rep, 4(5):233-242.
Garcia P S, Chieppa G, Desideri A, Cannata S, Romano E, Luly P, et al. (2012) Sticholysin II: a pore-forming toxin as a probe to recognize sphingomyelin in artificial and cellular membranes. Toxicon. October; 60(5):724-33.
Javaheri, et al. (2016). Helicobacter pylori adhesin HopQ engages in a virulence-enhancing interaction with human CEACAMs. Nature Microbiology 2, 16189.
Johnsson, N., George, N., and Johnsson, K. (2005). Protein chemistry on the surface of living cells. Chembiochem: a European journal of chemical biology 6, 47-52.
Kessler et al. (2017). The three-finger toxin fold: a multifunctional structural scaffold able to modulate cholinergic functions. J Neurochem. 142 Suppl 2:7-18.
King I. C., Gleixner, J., Doyle, L., Kuzin, A., Hunt, J. F., Xiao, R., Montelione, G. T., Stoddard, B. L., DiMaio, F., and Baker, D. (2015). Precise assembly of complex beta sheet topologies from de novo designed building blocks. eLife 4:e11012. doi: 10.7554/eLife.11012.
Kini R. M and Doley R. (2010) Structure, function and evolution of three-finger toxins: Mini proteins with multiple targets. Toxicon 56: 855-867.
Koide, S. (2009). Engineering of recombinant crystallization chaperones. Curr Opin Struct Biol 19(4): 449-457.
Martin A C. (2000). The ups and downs of protein topology; rapid comparison of protein structure. Protein Eng. 13(12):829-37.
Nogales, E. (2016). The development of cryo-EM into a mainstream structural biology technique. Nature Methods 13, 24-27.
Orengo et al. (1994). Protein superfamilies and domain superfolds. Nature. 15; 372(6507):631-4.
Pardon, E., Laeremans, T., Triest, S., Rasmussen, S. G., Wohlkonig, A., Ruf, A., Muyldermans, S., Hol, W. G., Kobilka, B. K., and Steyaert, J. (2014). A general protocol for the generation of Nanobodies for structural biology. Nature Protocols. 9: 674-693.
Rakestraw J, Sazinsky S, Piatesi A, Antipov E, Wittrup K. (2009). Directed evolution of a secretory leader for the improved expression of heterologous proteins and full-length antibodies in Saccharomyces cerevisiae. Biotechnol. Bioeng. 103, 1192-1201.
Rosso, J. P., et al. (2015). MmTX1 and MmTX2 from coral snake venom potently modulate GABA_{A} receptor activity. Proc Natl Acad Sci USA 112(8): E891-900.
Rudolph M J, Vance D J, Cassidy M S, Rong Y, Shoemaker C B, Mantis N J. (2016) Structural analysis of nested neutralizing and non-neutralizing B cell epitopes on ricin toxin's enzymatic subunit. Proteins: Structure, Function, and Bioinformatics. 1; 84(8):1162-72.
Shenkarev Z O, Shulepko M A, Peigneur S, Myshkin M Y, Berkut A A, Vassilevski A A, et al. (2019) Recombinant Production and Structure-Function Study of the Ts1 Toxin from the Brazilian Scorpion Tityus serrulatus. Dokl Biochem Biophys. Pleiades Publishing; January 1; 484(1):9-12.
Stepensky, 2018. Pharmacokinetics of Toxin-Derived Peptide Drugs. Toxins, 10, 483.
Uchariski T, Zogg T, Yin J, Yuan D, Wohlkonig A, Fischer B, et al. (2019) An improved yeast surface display platform for the screening of nanobody immune libraries. Scientific Reports. Nature Publishing Group; January 23; 9(1):1-12.

1. A functional fusion protein comprising a toxin fused with a scaffold protein, wherein the scaffold protein is a folded protein of at least 50 amino acids that interrupts the topology of the toxin at one or more accessible sites in an exposed β-turn of the toxin via two or more fusions, wherein the fusions are direct fusions or fusions made by a linker.

2. The functional fusion protein of claim 1, wherein the toxin comprises a β-strand-containing domain of at least three β-strands, and wherein the scaffold protein interrupts the topology of the β-strand-containing domain at one or more accessible sites in an exposed β-turn of the at least 3 β-strand-containing domain.

3. The functional fusion protein of claim 1, wherein the toxin is a venom toxin and wherein the scaffold protein is inserted in the exposed β-turn that connects β-strand β2 and β-strand (33 of said venom toxin.

4. The functional fusion protein of claim 1, wherein the toxin comprises a three-finger fold domain, and wherein the scaffold protein is inserted in the β-turn that connects β-strand β2 and β-strand β3 of the three-finger fold domain.

5. The functional fusion protein of claim 1, wherein the scaffold protein is a circularly permutated protein.

6. The functional fusion protein of claim 1, wherein the scaffold protein has a total molecular mass of at least 30 kDa.

7. A nucleic acid molecule encoding the functional fusion protein of claim 1.

8. The nucleic acid molecule of claim 7, wherein the nucleic acid molecule is comprised in a vector.

9. The nucleic acid molecule of claim 8, wherein the vector is optimized for expression in E. coli, for surface display in yeast, in phages, in bacteria, or in viruses.

10. The fusion protein of claim 1, wherein the functional fusion protein is comprised in a host cell.

11. The fusion protein of claim 10, wherein the functional fusion protein and a toxin receptor are co-expressed in the host cell.

12. The functional fusion protein of claim 1, wherein the functional fusion protein is present in a complex comprising:
(i) the functional fusion protein, and
(ii) a toxin target protein,
wherein the toxin target protein is specifically bound to the toxin part of the functional fusion protein.

13. A method for determining a 3-dimensional structure of a] functional fusion protein in complex with a toxin target protein, the method comprising:
(i) providing the complex of claim 12; and
(ii) displaying the complex in suitable conditions for structural analysis, wherein the 3D structure of the protein complex is determined at high-resolution.

14. (canceled)

15. The method according to claim 13, wherein determining the 3D structure of the protein complex comprises single particle cryo-EM or crystallography.

16. (canceled)
